(19) United States
(12) Patent Application Publication
(10) Pub. No.: US 2024/0368591 A1
(43) Pub. Date: Nov. 7, 2024

(54) OLIGOMERIC COMPOUND FOR DYSTROPHIN RESCUE IN DMD PATIENTS THROUGHOUT SKIPPING OF EXON-51

(71) Applicants: SQY THERAPEUTICS, NOISY-LE-ROI (FR); UNIVERSITE DE VERSAILLES SAINT-QUENTIN-EN-YVELINES, VERSAILLES Cedex (FR); INSTITUT NATIONAL DE LA SANTE ET DE LA RECHERCHE MEDICALE, PARIS (FR)

(72) Inventors: Luis GARCIA, NOISY-LE-ROI (FR); Aurélie GOYENVALLE, VOISINS-LE-BRETONNEUX (FR); Fedor SVINARTCHOUK, VILLEJUIF (FR); Graziella GRIFFITH, ELANCOURT (FR); Aurélie AVRIL-DELPLANQUE, JOUY-EN-JOSAS (FR)

(21) Appl. No.: 18/030,265

(22) PCT Filed: Oct. 4, 2021
(86) PCT No.: PCT/EP2021/077276

§ 371 Date: Apr. 4, 2023

(30) Foreign Application Priority Data
Oct. 5, 2020 (EP) 20200140.0

(51) Int. Cl. C12N 15/113; A61K 47/54; A61P 21/00

(52) U.S. Cl. CPC C12N 15/113; A61K 47/543; A61P 21/00; C12N 2310/11; C12N 2310/315; C12N 2310/321; C12N 2310/323; C12N 2310/3515; C12N 2320/33

(57) ABSTRACT

Disclosed is an oligomeric compound comprising from 10 to 50 monomer subunits, at least part of the sequence of which is complementary to the following sequence: AAGGAAACUGCCAUCUCCAA (SEQ ID NO: 1 in the appended sequence listing). Also disclosed is a pharmaceutical composition comprising said oligomeric compound and use for treating Duchenne Muscular Dystrophy.

### CROSS-REFERENCE TO RELATED APPLICATIONS

This application is a national stage entry of PCT International Application No. PCT/EP2021/077276 filed on Oct. 4, 2021, which claims the benefit of EP 20200140.0 filed Oct. 5, 2020, each of which is incorporated by reference in its entirety.

### SEQUENCE LISTING

The instant application contains a Sequence Listing which has been submitted electronically in .TXT format and is hereby incorporated by reference in its entirety. Said .TXT copy, created on Aug. 15, 2023, is named “117212-5001-US SeqListing 15 Aug. 2023” and is 5,036 bytes in size.

### TECHNICAL FIELD

The present invention belongs to the general technical field of therapeutic nucleic acid molecules and notably of therapeutic nucleic acid molecules useful for restoring dystrophin activity using splice-switching technology in patients with Duchenne muscular dystrophy (DMD).

More particularly, the present invention provides new oligomeric compounds possibly containing one or more tricyclo-deoxyribonucleic acid (tc-DNA) nucleosides and one or more lipid moieties covalently linked to said oligomeric compound either directly or via a spacer, for targeting the exon 51 of the human dystrophin gene.

These oligomeric compounds and, in particular, the one designed hereinafter as SQY51, meet the therapeutic needs of over ten percent of DMD patients with large deletions; they are compatible with systemic delivery, they access the whole musculature including heart and smooth muscles, they cross the blood brain barrier while displaying little bioaccumulation.

### BACKGROUND

Antisense technology is an effective means for reducing the expression of specific gene products and can therefore be useful in therapeutic, diagnostic, and research applications. Generally, the principle behind antisense technology is that an antisense compound (a sequence of nucleotides or analogues thereof) hybridizes to a target nucleic acid and modulates gene expression activities or function, such as transcription and/or translation. Regardless of the specific mechanism, its sequence-specificity makes antisense compounds attractive as tools for target validation and gene functionalization, as well as therapeutics to selectively modulate the expression of genes involved in the pathogenesis of diseases.

### BRIEF SUMMARY

In one aspect, the invention concerns an oligomeric compound comprising from 10 to 50 monomer subunits, at least part of the sequence of which is complementary to the following sequence: AAGGAAACUGCCAUCUCCAA (SEQ ID NO: 1 in the appended sequence listing). In some embodiments, at least part of the sequence of said oligomeric compound is complementary to the sequence corresponding to the region +48+62 of SEQ ID NO: 2 in the appended sequence listing. In some embodiments, the oligomeric compound comprises or consists of an antisense oligonucleotide. In some embodiments, the oligomeric compound comprises at least one nucleotide sequence having at least 70% identity with the following tc-DNA nucleotide sequence: GGAGATGGCAGTTTC (SEQ ID NO: 3 in the appended sequence listing). In some embodiments, the oligomeric compound comprises or consists of a tricyclo-DNA antisense oligonucleotide. In some embodiments, the oligomeric compound comprises or consists of a tricyclo-phosphorothioate DNA antisense oligonucleotide. In some embodiments, the oligomeric compound comprises one or more tricyclo-deoxyribonucleic acid (tc-DNA) nucleosides and at least one modified ribonucleic acid nucleoside. In some embodiments, said modified ribonucleic acid nucleoside is a 2′-O-methyl RNA nucleoside. In some embodiments, the monomer subunits of said oligomeric compound are joined by phosphodiester internucleoside linkages. In some embodiments, the oligomeric compound comprises or consists of a nucleotide sequence corresponding to one of the following nucleotide sequences:
5′-GGAGATgGCAGTTTC-3′ (SEQ ID NO: 4 in the appended sequence listing),
5′-GGAGATGgCAGTTTC-3′ (SEQ ID NO: 5 in the appended sequence listing),
5′-GGAGATGGcAGTTTC-3′ (SEQ ID NO: 6 in the appended sequence listing), and
5′-GGAGATGGCaGTTTC-3′ (SEQ ID NO: 7 in the appended sequence listing). in which tcDNA nucleotides are typed in capital letters while the modified ribonucleic acid nucleoside is typed in lowercase letter. In some embodiments, wherein the oligomeric compound is combined with one or more lipid moieties. In some embodiments, the oligomeric compound is selected from the group consisting of:
palmitate-NH—C₆alkylene-OP(═S)(OH)-GGAGATgGCAGTTTC-3′ (SEQ ID NO: 4 in the appended sequence listing),
palmitate-NH—C₆alkylene-OP(═S)(OH)-GGAGATGgCAGTTTC-3′ (SEQ ID NO: 5 in the appended sequence listing),
palmitate-NH—C₆alkylene-OP(═S)(OH)-GGAGATGGcAGTTTC-3′ (SEQ ID NO: 6 in the appended sequence listing), and
palmitate-NH—C₆alkylene-OP(═S)(OH)-GGAGATGGCaGTTTC-3′ (SEQ ID NO: 7 in the appended sequence listing).

In another aspect, the invention concerns a pharmaceutical composition comprising, as an active ingredient, an oligomeric compound of the disclosure, and a pharmaceutically acceptable vehicle.

In another aspect, the invention concerns an oligomeric compound disclosed herein or a pharmaceutical composition disclosed herein for use in treating Duchenne Muscular Dystrophy in a patient in need.

In another aspect, the invention concerns a method of treating Duchenne Muscular Dystrophy in a patient in need. In some embodiments, the method includes administering a therapeutically effective amount of an oligomeric compound disclosed herein or a pharmaceutical composition disclosed herein to the patient.

### DESCRIPTION

The present invention includes therapeutic nucleic acid molecules useful for splice-switching technology in patients with DMD. More particularly, the present invention includes therapeutic nucleic acid molecules which do not present the drawbacks of molecules known in the art, such as toxicity, and usable for restoring a semi-functional dystrophin.

Unless defined otherwise, all technical and scientific terms used herein have the same meanings as commonly understood by those skilled in the art to which this invention belongs. The headings used herein are solely for convenience reasons and should not be construed as limiting for the disclosure of any of the aspects and embodiments of the present invention.

The expression “oligomeric compound” and the term “oligonucleotide” refer to a synthetic compound comprising from 10 to 50 monomer subunits linked by internucleosidic linkage groups. The length of an oligonucleotide may be denoted by the number of monomer subunits concatenated or linked together to the term “-mer”. For example, an oligonucleotide containing ten monomer subunits is a 10-mer (or decamer), and an oligonucleotide containing 25 monomer subunits is a 25-mer. Oligonucleotides and oligomeric compounds of the present invention are listed from left to right following the order of the 5′ to the 3′ end, respectively. In some embodiments, at least two of said 10 to 50 monomer subunits are tricyclo-deoxyribonucleic acid (tc-DNA) nucleosides. In some embodiments, are independently selected from naturally occurring nucleosides, modified nucleosides or nucleoside mimetics. The oligomeric compound can be single stranded or double stranded. In one embodiment, the oligomeric compound is double stranded (i.e., a duplex). In some embodiments, the oligomeric compound is single stranded.

The expression “monomer subunits”, as used herein, is meant to include all manner of monomer units that are amenable to oligomer synthesis including, and typically referring to monomer subunits such as α-D-ribonucleosides, β-D-ribonucleosides, α-D-2′-deoxyribonucleosides, β-D-2′-deoxyribonucleosides, naturally occurring nucleosides, modified nucleosides, and hereby in particular tricyclo-deoxyribonucleic acid (tc-DNA) nucleosides, 2′-modified ribonucleic acid (2′-modified-RNA) nucleosides, locked nucleic acid (LNA) nucleosides, peptide nucleic acids (PNAs) nucleosides, 2′-deoxy 2′-fluoro-arabino nucleosides, hexitol nucleic acids (HNAs) nucleosides; and phosphorodiamidate morpholino (PMO) nucleosides, mimetics of nucleosides, naturally occurring nucleotides, modified nucleotides, and hereby in particular tricyclo-deoxyribonucleic acid (tc-DNA) nucleotides and 2′-modified ribonucleic acid (2′-modified-RNA) nucleotides, and mimetics of nucleotides. Advantageously, the expression “monomer subunit”, as used herein, refers to naturally occurring nucleosides and modified nucleosides, and hereby in particular to ribonucleosides, deoxyribonucleosides, tricyclo-deoxyribonucleic acid (tc-DNA) nucleosides, 2′-modified ribonucleic acid (2′-modified-RNA) nucleosides, locked nucleic acid (LNA) nucleosides, peptide nucleic acids (PNAs) nucleosides, 2′-deoxy-2′-fluoro-arabinonucleosides, hexitol nucleic acids (HNAs) nucleosides and phosphorodiamidate morpholino (PMO) nucleosides, and to naturally occurring nucleotides and modified nucleotides, and hereby in particular to ribonucleotides, deoxyribonucleotides, tricyclo-deoxyribonucleic acid (tc-DNA) nucleotides, 2′-modified ribonucleic acid (2′-modified-RNA) nucleotides, locked nucleic acid (LNA) nucleotides, peptide nucleic acids (PNAs) nucleotides, 2′-deoxy-2′-fluoro-arabinonucleotides, hexitol nucleic acids (HNAs) nucleotides and phosphorodiamidate morpholino (PMO) nucleotides. More particularly, the expression “monomer subunit”, as used herein, refers to modified nucleotides, and hereby in particular tricyclo-deoxyribonucleic acid (tc-DNA) nucleotides and 2′-modified ribonucleic acid (2′-modified-RNA) nucleotides.

The term “base analog”, also referred to as “modified nucleobase”, refers to chemical modifications of DNA or RNA bases with a molecular structure that mimics natural DNA or RNA bases. Base analogs include, but are not limited to, 5-methylcytosine, 5-bromouracil, inosine, 5-substituted pyrimidines, 6-azapyrimidines and N-2, N-6 and 0-6 substituted purines, including 2-aminopropyladenine, 5-propynyluracil and 5-propynylcytosine. Base analogs also include, but are not limited to, 5-hydroxymethylcytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine, 2-thiocytosine, 5-halouracil and cytosine, 5-propinyluracil and 5-propinylcytosine (and other alkynyl derivatives of pyrimidine bases), 6-azouracil, 6-azocytosine, 6-azothymine, 5-uracil (pseudouracil), 4-thiouracil, 8-halo, 8-amino, 8-thiol, 8-thioalkyl, 8-hydroxyl and other 8-substituted adenines and guanines, 5-halo and particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deaza-adenine, 3-deazaguanine and 3-deaza-adenine, universal bases, tricyclic pyrimidines such as phenoxazine cytidine(1H-pyrimido [5,46][l,4]benzoxazin-2(3H)-one), phenothiazine cytidine (1H-pyrimido[5,4-6][1,4]benzothiazine-2(3H)-one), G-clamps such as a substituted phenoxazine cytidine (e.g. 9-(2-aminoethoxy)-H-pyrimido[5,4-6][1,4]benzoxazin-2(3H)-one), carbazole cytidine (2H-pyrimido[4,5-b]indol-2-one), and pyridoindole cytidine (2H-pyrido[3′,2′:4,5]pyrrolo[2,3-<f]pyrimidin-2-one). Base analogs may also include those in which the purine or pyrimidine base is replaced with other heterocycles, for example 7-deaza-adenine, 7-deazaguanosine, 2-aminopyridine and 2-pyridone. The preparation of modified nucleobases is known in the art.

The term “complementary”, as used herein, refers to a nucleic acid sequence that can form hydrogen bond(s) with another nucleic acid sequence by either traditional Watson Crick base pairing or other non-traditional types of pairing (e.g., Hoogsteen or reversed Hoogsteen hydrogen bonding) between complementary nucleosides or nucleotides. “Complementary” (or “specifically hybridizable”) are terms that indicate a sufficient degree of complementarity or precise pairing such that stable and specific binding occurs between an oligomeric compound and a pre-mRNA or mRNA target.

It is understood in the art that a nucleic acid molecule need not be 100% complementary to a target nucleic acid sequence to be complementary. That is, two nucleic acid molecules may be less than fully complementary. Complementarity is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing of 5, 6, 7, 8, 9, or 10 nucleotides between the first and second nucleic acid molecules represents 50%, 60%, 70%, 80%, 90%, and 100% complementarity, respectively. “Fully” complementary nucleic acid molecules means those in which all the contiguous residues of a first nucleic acid molecule form hydrogen bonds with the same number of contiguous residues in a second nucleic acid molecule, wherein the nucleic acid molecules either both have the same number of nucleotides (i.e., have the same length) or the two molecules have different lengths.

The expression “antisense oligonucleotide” refers to a single strand of DNA or RNA or oligomeric compound that is complementary to a targeted sequence. An antisense oligonucleotide is capable of hybridizing to a pre-mRNA or a mRNA having a complementary coding or non-coding nucleotide sequence. In the present case, this targeted nucleotide sequence corresponds to sequence SEQ ID NO: 1 in the appended sequence listing and typically to the sequence corresponding to the region +48+62 of SEQ ID NO: 2 in the appended sequence listing.

By “identity percent” between two nucleotide sequences (or between two amino acid sequences), it is meant, within the scope of the present disclosure, a percent of identical nucleotide (or amino acid) residues between the two sequences being compared, this percent being obtained after implementing the best alignment (optimum alignment) between both sequences. Those skilled in the art know different techniques enabling such an identity percent to be obtained and involving homology algorithms or computer programs such as the program BLAST.

The term “tricyclo-DNA” (tc-DNA) refers to a class of constrained oligodeoxyribonucleotide analogs in which each nucleotide is modified by the introduction of a cyclopropane ring to restrict conformational flexibility of the backbone and to optimize the backbone geometry of the torsion angle Y. In detail, the tc-DNA differs structurally from DNA by an additional ethylene bridge between the centers C(3′) and C(5′) of the nucleosides, to which a cyclopropane unit is fused for further enhancement of structural rigidity.

The term “internucleosidic linkage group” as used herein, refers to any linkage group known in the art that is able to link, preferably links, said tricyclo-deoxyribonucleic acid (tc-DNA) nucleoside either to a further tc-DNA nucleoside, a nucleoside other than a tc-DNA nucleoside, a non-nucleoside including a peptide, protein. Representative patents that teach such possible linkage groups are without limitation U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; 5,677,439; 5,646,269 and 5,792,608. The term “internucleosidic linkage group”, thus, includes phosphorus linkage groups and non-phosphorus linkage groups. Non-phosphorus linkage groups do not contain a phosphorus atom and examples of non-phosphorus linkage groups include, and are typically and preferably selected from alkyl, aryl, preferably, phenyl, benzyl, or benzoyl, cycloalkyl, alkylenearyl, alkylenediaryl, alkoxy, alkoxyalkylene, alkylsulfonyl, alkyne, ether, each independently of each other optionally substituted with cyano, nitro, halogen; carboxyl, amide, amine, amino, imine, thiol, sulfide, sulfoxide, sulfone, sulfamate, sulfonate, sulfonamide, siloxane or mixtures thereof. Typically, and preferably, said internucleosidic linkage group is a phosphorus linkage group, and said phosphorus linkage group refers to a moiety comprising a phosphorus atom in the P^{III} or P^{V} valence state. Further preferably, said internucleosidic linkage group is a phosphorus linkage group. Again further preferably, said internucleosidic linkage group is selected from a phosphodiester linkage group, a phosphotriester linkage group, a phosphorothioate linkage group, a phosphorodithioate linkage group, a phosphonate linkage group, preferably a H-phosphonate linkage group or a methylphosphonate linkage group; a phosphonothioate linkage group, preferably a H-phosphonothioate linkage group, a methyl phosphonothioate linkage group; a phosphinate linkage group, a phosphorthioamidate linkage, a phosphoramidate linkage group, or a phosphite linkage group. In some embodiments, said internucleosidic linkage group is selected from a phosphodiester linkage group, a phosphotriester linkage group, a phosphorothioate linkage group, or a phosphonate linkage group, wherein the phosphonate is preferably a H-phosphonate linkage group or methylphosphonate linkage group.

As used herein, the term “nucleoside” refers to a compound comprising a nucleobase and a sugar covalently linked to said nucleobase. Further, the term “nucleoside” is meant to include all manner of naturally occurring or modified nucleosides or nucleoside mimetics that can be incorporated into an oligomer using natural or chemical oligomer synthesis. Typically, and preferably, the term “nucleoside”, as used herein, refers to a naturally occurring nucleoside, a modified nucleoside or nucleoside mimetic. The term “modified nucleosides” is intended to include modifications made to the sugar and/or nucleobase of a nucleoside as known to the skilled person in the art and described herein.

The term “nucleoside mimetic” is intended to include those structures used to replace the sugar and the nucleobase. Examples of nucleoside mimetics include nucleosides wherein the nucleobase is replaced with a phenoxazine moiety (for example the 9-(2-aminoethoxy)-1,3-diazaphenoxazine-2-one group) and the sugar moiety is replaced a cyclohexenyl or a bicyclo[3.1.0]hexyl moiety. The term “nucleoside” also includes combinations of modifications, such as more than one nucleobase modification, more than one sugar modification or at least one nucleobase and at least one sugar modification.

The sugar of the nucleoside includes without limitation a monocyclic, bicyclic or tricyclic ring system, preferably a tricyclic or bicyclic system or a monocyclic ribose or de(s)oxyribose. Modifications of the sugar further include but are not limited to modified stereochemical configurations, at least one substitution of a group or at least one deletion of a group. A modified sugar is typically and preferably a modified version of the ribosyl moiety as naturally occurring in RNA and DNA (i.e., the furanosyl moiety), such as bicyclic sugars, tetrahydropyrans, 2′-modified sugars, 3′-modified sugars, 4′-modified sugars, 5′-modified sugars, or 4′-substituted sugars. Examples of suitable sugar modifications are known to the skilled person and include, but are not limited to 2′, 3′ and/or 4′ substituted nucleosides (e.g. 4′-S-modified nucleosides); 2′-O-modified RNA nucleotide residues, such as 2′-O-alkyl or 2′-O-(substituted)alkyl e.g. 2′-O-methyl, 2′-O-(2-cyanoethyl), 2′-O-(2-methoxy)ethyl (2′-MOE), 2′-O-(2-thiomethyl)ethyl; 2′-O-(haloalkoxy)methyl e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl](MOCE), 2′-O-[2-(N-methylcarbamoyl)ethyl](MCE), 2′-O-[2-(N,N-dimethylcarbamoyl)ethyl](DMCE), in particular a 2′-O-methyl modification or a 2′-O-methoxyethyl (2′-O-MOE); or other modified sugar moieties, such as morpholino (PMO), cationic morpholino (PMOPIus) or a modified morpholino group, such as PMO-X. The term “PMO-X” refers to a modified morpholino group comprising at least one 3′ or 5′ terminal modification, such 3′-fluorescent tag, 3′ quencher (e.g. 3′-carboxyfluorescein, 3′-Gene Tools Blue, 3′-lissamine, 3′-dabcyl), 3′-affinity tag and functional groups for chemical linkage (e.g. 3′-biotin, 3′-primary amine, 3′-disulfide amide, 3′-pyridyl dithio), 5′-end modifications (5′-primary amine, 5′-dabcyl), 3′-azide, 3′-alkyne, 5′-azide, 5′-alkyne, or as disclosed in WO2011/150408 and US2012/0065169.

“Bicylic sugar moieties” comprise two interconnected ring systems, e.g. bicyclic nucleosides wherein the sugar moiety has a 2′-O—CH(alkyl)-4′ or 2′-O—CH2-4′ group, locked nucleic acid (LNA), xylo-LNA, alpha-L-LNA, beta-D-LNA, cEt (2′-0,4′-C constrained ethyl) LNA, cMOEt (2′-0,4′-C constrained methoxyethyl) LNA, ethylene-bridged nucleic acid (ENA), hexitol nucleic acid (HNA), fluorinated HNA (F-HNA), pyranosyl-RNA (p-RNA), or 3′-deoxypyranosyl-DNA (p-DNA).

The term “lipid moiety” as used herein refers to moieties that are derived from, typically and advantageously, hydrocarbons, oils, fats (such as fatty acids, glycerides), sterols, steroids, and derivative forms of these compounds. Suitable lipid moieties include moieties derived from fatty acids and their derivatives, hydrocarbons and their derivatives, and sterols, such as cholesterol. As used herein, the term lipid moiety also includes amphipathic compound moieties, which contain both lipid and hydrophilic moieties.

The term “hydrocarbon”, as used herein, encompasses compounds that consist only of hydrogen and carbon, joined by covalent bonds. The term encompasses open chain (aliphatic) hydrocarbons, including straight (unbranched) chain and branched hydrocarbons, and saturated as well as mono- and polyunsaturated hydrocarbons. The term also encompasses hydrocarbons containing one or more aromatic ring, preferably the term excludes hydrocarbons containing one or more aromatic ring. The terms “straight” and “unbranched”, are interchangeably used herein.

The term “fatty acid”, as used herein, refers to a hydrocarbon chain that terminates with a carboxylic acid group, wherein said hydrocarbon chain is typically and preferably either an alkyl or alkenyl of typically 3 to 32 carbons long, and that are, thus, saturated or unsaturated, and that are optionally substituted by one or more, preferably one, carboxylic group (—COOH), one or more, preferably one, C₁₋₃₂ alkyl, one or more, preferably one, phosphate group (HOP(O)(OH)O—), one or more, preferably one, phosphonate group (HOP(O)O—), one or more, preferably one, thiophosphate group (HOP(O)(SH)O—), one or more, preferably one, dithiophosphate group (HOP(S)(SH)O—), one or more, preferably one, diphosphate group (HO—P(O)(OH)—O—P(O)(OH)—O—), one or more, preferably one, triphosphate group (HO—P(O)(OH)—O—P(O)(OH)—O—P(O)(OH)—O—), one or more phenyl group (—C₆H₅), one or more phenyl group substituted with a halogen, preferably iodine, or a carboxylic group. If a fatty acid contains one or more double bond, and is thus unsaturated, there is the possibility of either a cis or trans geometric isomerism. The term “fatty acid moiety”, as used herein, refers to a moiety derived from a fatty acid, as defined herein, wherein one carboxylic group (—COOH) of said fatty acid becomes and is a —C(O)— group of said fatty acid moiety, which —C(O)— group is linked to said oligonucleotide either directly or via spacer in accordance with the present invention. Preferably, the term “fatty acid” as used herein refers to a hydrocarbon chain that terminates with a carboxylic acid group, wherein said hydrocarbon chain is typically and preferably either an alkyl or alkenyl of typically 3 to 32 carbons long, and that are, thus, saturated or unsaturated, and that are optionally substituted by one or more, preferably one, carboxylic group (—COOH), one or more, preferably one, C₁₋₃₂ alkyl, one or more, preferably one, phosphate group (HOP(O)(OH)O—), one or more, preferably one, phosphonate group (HOP(O)O—), one or more, preferably one, thiophosphate group (HOP(O)(SH)O—), one or more, preferably one, dithiophosphate group (HOP(S)(SH)O—), one or more, preferably one, diphosphate group (HO—P(O)(OH)—O—P(O)(OH)—O—), one or more, preferably one, triphosphate group (HO—P(O)(OH)—O—P(O)(OH)—O—P(O)(OH)—O—), one or more phenyl group (—C₆H₅), one or more phenyl group substituted with a halogen, preferably iodine, or a carboxylic group. Preferably, said fatty acid has an even numbers of carbon atoms, wherein the carbon atom of the carboxylic group (—COOH) of said fatty acid or said —C(O)— group of said fatty acid moiety is included in the counting of the numbers of carbon atoms.

Thus, fatty acids preferably contain even or uneven numbers, preferably even numbers, of carbon atoms in a straight chain (commonly 3-32 carbons) and can be saturated or unsaturated, and can contain, or be modified to contain, a variety of substituent groups, preferably by one or more, preferably one, carboxylic group (—COOH), one or more, preferably one, C₁₋₃₂ alkyl, one or more, preferably one, phosphate group (HOP(O)(OH)O—), one or more, preferably one, phosphonate group (HOP(O)O—), one or more, preferably one, thiophosphate group (HOP(O)(SH)O—), one or more, preferably one, dithiophosphate group (HOP(S)(SH)O—), one or more, preferably one, diphosphate group (HO—P(O)(OH)—O—P(O)(OH)—O—), one or more, preferably one, triphosphate group (HO—P(O)(OH)—O—P(O)(OH)—O—P(O)(OH)—O—), one or more phenyl group (—C₆H₅), one or more phenyl group substituted with a halogen, preferably iodine, or a carboxylic group.

The term “fatty diacid” refers to fatty acids as defined herein but with an additional carboxylic acid group in the omega position. Thus, fatty diacids are dicarboxylic acids. The term “fatty diacid moiety”, as used herein, refers to a moiety derived from a fatty diacid, as defined herein, wherein one carboxylic group (—COOH) of said fatty diacid becomes and is a —C(O)— group of said fatty diacid moiety, which —C(O)— group is linked to said oligonucleotide either directly or via spacer in accordance with the present invention. Preferred embodiments of fatty diacids are saturated fatty diacids optionally substituted by one or more, preferably one, C₁₋₃₂ alkyl, one or more, preferably one, phosphate group (HOP(O)(OH)O—), one or more, preferably one, phosphonate group (HOP(O)O—), one or more, preferably one, thiophosphate group (HOP(O)(SH)O—), one or more, preferably one, dithiophosphate group (HOP(S)(SH)O—), one or more, preferably one, diphosphate group (HO—P(O)(OH)—O—P(O)(OH)—O—), one or more, preferably one, triphosphate group (HO—P(O)(OH)—O—P(O)(OH)—O—P(O)(OH)—O—), one or more, preferably one, phenyl group (—C₆H₅), one or more, preferably one, phenyl group substituted with a halogen, preferably iodine, or a carboxylic group. Preferred examples include glutaric acid optionally substituted by one C₆₋₂₄alkyl such 3-pentadecylglutaric acid (PDG).

The term “alkylphosphate moiety” as used herein refers to groups of C₃₋₃₂alkyl-O—P(O)(OH)—O—, wherein said C₃₋₃₂alkyl is independently selected from C₃₋₃₂alkyl as defined herein.

The term “alkylphosphonate moiety” as used herein refers to groups of C₃₋₃₂alkyl-O—P(O)—O—, wherein said C₃₋₃₂alkyl is independently selected from C₃₋₃₂alkyl as defined herein.

The term “alkyl”, as used herein, refers to a straight or branched hydrocarbon chain radical consisting solely of carbon and hydrogen atoms, containing no unsaturation, having from one to thirty-two carbon atoms (e.g., (C₁₋₃₂)alkyl or C₁₋₃₂ alkyl), and which may be or typically is attached to the rest of the molecule by a single bond. Whenever it appears herein, a numerical range such as “1 to 32” refers to each integer in the given range. For example, “1 to 32 carbon atoms” means that the alkyl group may consist of 1 carbon atom, 2 carbon atoms, 3 carbon atoms, etc., up to and including 32 carbon atoms, although the definition is also intended to cover the occurrence of the term “alkyl” where no numerical range is specifically designated. Typical alkyl groups include, but are not limited to, methyl, ethyl, n-propyl, 1-methylethyl (interchangeably used with iso-propyl; interchangeably abbreviated herein as iPr or Pri), n-butyl, isobutyl, sec-butyl, isobutyl, tertiary butyl (interchangeably used with 1,1-dimethylethyl or tert-butyl), n-pentyl, isopentyl, neopentyl, hexyl, septyl, octyl, nonyl and decyl. Unless stated otherwise specifically in the specification, an alkyl group is optionally substituted by one or more of substituents which are independently alkenyl, alkoxy, carboxylic group (—COOH), heteroalkyl, heteroalkenyl, hydroxyl, phosphate group (—OP(O)(OH)O—), phosphonate group (—OP(O)O—), phenyl group (—C₆H₄) optionally substituted with a halogen, preferably iodine, or a carboxylic group. Preferably, the term “alkyl”, as used herein, refers to an unsubstituted alkyl as defined herein.

The term “alkylene”, as used herein, refers to a straight or branched hydrocarbon chain bi-radical derived from alkyl, as defined herein, wherein one hydrogen of said alkyl is cleaved off generating the second radical of said alkylene. Examples of alkylene are, by way of illustration, —CH₂—, —CH₂—CH₂—, —CH(CH₃)—, —CH₂—CH₂—CH₂—, —CH(CH₃)—CH₂—, or —CH(CH₂CH₃)—.

The term “alkenyl”, as used herein, refers to a straight or branched hydrocarbon chain radical group consisting solely of carbon and hydrogen atoms, containing at least one double bond, and having from 3 to 32 carbon atoms (i.e., (C₃₋₃₂)alkenyl or C₃₋₃₂ alkenyl), which may be or typically is attached to the rest of the molecule by a single bond. Whenever it appears herein, a numerical range such as “3 to 32” refers to each integer in the given range—e.g., “3 to 32 carbon atoms” means that the alkenyl group may consist of 3 carbon atoms, 4 carbon atoms, etc., up to and including 32 carbon atoms. Typical alkenyl groups include, but are not limited to ethenyl (i.e., vinyl), prop-1-enyl (i.e., allyl), but-1-enyl, pent-1-enyl and penta-1,4-dienyl. Each double bond can be of either the (E)— or (Z)-configuration. Alkenyl, thus, may include, if applicable, either each of said double bond in its (E)-configuration, in its (Z)-configuration and mixtures thereof in any ratio. Unless stated otherwise specifically in the specification, an alkenyl group is optionally substituted by one or more of substituents which are independently alkenyl, alkoxy, carboxylic group (—COOH), heteroalkyl, heteroalkenyl, hydroxyl, phosphate group (—OP(O)(OH)O—), phosphonate group (—OP(O)O—), phenyl group (—C₆H₄) optionally substituted with a halogen, preferably iodine, or a carboxylic group. Preferably, the term “alkenyl”, as used herein, refers to an unsubstituted alkenyl as defined herein.

The term “alkenylene”, as used herein, refers to a straight or branched hydrocarbon chain bi-radical derived from alkenyl, as defined herein, wherein one hydrogen of said alkenyl is cleaved off generating the second radical of said alkenylene.

The term “alkynyl” refers to a straight or branched hydrocarbon chain radical group consisting solely of carbon and hydrogen atoms, containing at least one triple bond, having from two to ten carbon atoms (i.e., (C₂₋₃₂)alkynyl or C₂₋₃₂ alkynyl). Whenever it appears herein, a numerical range such as “2 to 32” refers to each integer in the given range—e.g., “2 to 32 carbon atoms” means that the alkynyl group may consist of 2 carbon atoms, 3 carbon atoms, etc., up to and including 32 carbon atoms. Typical alkynyl groups include, but are not limited to ethynyl, propynyl, butynyl, pentynyl and hexynyl. Unless stated otherwise specifically in the specification, an alkynyl group is optionally substituted by one or more of substituents which are independently alkenyl, carboxylic group (—COOH), heteroalkyl, heteroalkenyl, phosphate group (—OP(O)(OH)O—), phosphonate group (—OP(O)O—), phenyl group (—C₆H₄) optionally substituted with a halogen, preferably iodine, or a carboxylic group. Preferably, the term “alkynyl”, as used herein, refers to an unsubstituted alkynyl as defined herein.

The term “alkynylene”, as used herein, refers to a straight or branched hydrocarbon chain bi-radical derived from alkynyl, as defined herein, wherein one hydrogen of said alkynyl is cleaved off generating the second radical of said alkynylene. The term “alkoxy” refers to the group —O-alkyl, including from 1 to 32 carbon atoms of a straight, branched configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons, also referred to as (C₁₋₆)alkoxy or O—C₁₋₆alkyl.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more of substituents which are independently alkenyl, carboxylic group (—COOH), heteroalkyl, heteroalkenyl, phosphate group (—OP(O)(OH)O—), phosphonate group (—OP(O)O—), phenyl group (—C₆H₄) optionally substituted with a halogen, preferably iodine, or a carboxylic group.

The term “acyl” refers to the groups (alkyl)—C(O)—, (aryl)—C(O)—, (heteroaryl)—C(O)—, and (heteroalkyl)—C(O)—, wherein the group is attached to the parent structure through the carbonyl functionality. Unless stated otherwise specifically in the specification, the alkyl, aryl or heteroaryl moiety of the acyl group is optionally substituted by one or more of substituents which are independently alkenyl, carboxylic group (—COOH), heteroalkyl, heteroalkenyl, phosphate group (—OP(O)(OH)O—), phosphonate group (—OP(O)O—), phenyl group (—C₆H₄) optionally substituted with a halogen, preferably iodine, or a carboxylic group.

The terms “amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6— or 7-membered ring. For example, —N(R^{a})₂ is intended to include, but is not limited to, 1-pyrrolidinyl and 4-morpholinyl. Unless stated otherwise specifically in the specification, an amino or amine group is optionally substituted by one or more of substituents which are independently alkenyl, carboxylic group (—COOH), heteroalkyl, heteroalkenyl, phosphate group (—OP(O)(OH)O—), phosphonate group (—OP(O)O—), phenyl group (—C₆H₄) optionally substituted with a halogen, preferably iodine, or a carboxylic group.

The terms “aromatic” or “aryl” or “Ar” refers to an aromatic radical with six to ten ring atoms (e.g., C₆-C₁₀ aromatic or C₆-C₁₀aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups.

The terms “aralkyl” or “arylalkyl” refers to an (aryl)alkyl-radical where aryl and alkyl are as disclosed herein, and which are optionally substituted by one or more of the substituents described as suitable substituents for aryl and alkyl respectively.

The term “carboxyl” or “carboxylic”, as interchangeably used herein, refers to a —(C═O)OH radical.

The term “cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like.

The term “fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

The term “halogen”, as used herein, refers to fluorine, chlorine, bromine, or iodine, preferably iodine. In some embodiments, the halogen substituent is iodine.

The terms “heteroalkyl,” and “heteroalkenyl”, as used herein, refer to optionally substituted alkyl and alkenyl radicals and which have one or more skeletal chain atoms selected from an atom other than carbon, e.g., oxygen, nitrogen, sulfur, phosphorus or combinations thereof. A numerical range may be given, e.g., C₁-C₄ heteroalkyl, which refers to the chain length in total, which in this example is 4 atoms long.

The terms “heteroaryl” or “heteroaromatic” or “HetAr” refers to a 5- to 18-membered aromatic radical (e.g., C₅-C₁₃heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene.

The term “stereoisomers” refers to compounds, which have identical chemical constitution, but differ with regard to the arrangement of the atoms or groups in space.

“Diastereomer” refers to a stereoisomer with two or more centers of chirality in which the compounds are not mirror images of one another. Diastereomers have different physical properties, e.g. melting points, boiling points, spectral properties, and chemical and biological reactivities. Mixtures of diastereomers may be separated under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McRaw-Hiff Dictionary of Chemical Terms (1984), McGraw-Hill Book Company, New York; and Eliel, E. and Wilen, S., “Stereochemistry of Organic Compounds”, John Wiley & Sons, Inc., New York, 1994.

The symbols (*), (#) and (§ ) in a chemical formula designates i) a point of attachment, ii) a radical, and/or iii) an unshared electron.

The term “antisense oligonucleotide (AON)”, as used herein, refers to an oligonucleotide or oligomeric compound that is capable of interacting with and/or hybridizing to a pre-mRNA or an mRNA having a complementary nucleotide sequence thereby modifying gene expression.

The term “protecting group”, as used herein, is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in Protecting Groups in Organic Synthesis, T. W. Greene and P. G. M. Wuts, 3^{rd} edition, John Wiley & Sons, New York 1999.

The terms “protecting group for an amino”, “protecting group for an amino group”, or “amino protecting group” as interchangeably used herein, are well known in the art and include those described in detail in Protecting Groups in Organic Synthesis, T. W. Greene and P. G. M. Wuts, 3^{rd} edition, John Wiley & Sons, New York (1999), Greene's Protective Groups in Organic Synthesis, P. G. M. Wuts, 5^{th} edition, John Wiley & Sons, (2014), and in Current Protocols in Nucleic Acid Chemistry, edited by S. L. Beaucage et al. 06/2012, and hereby in particular in Chapter 2. Suitable “amino protecting groups” for the present invention include and are typically and preferably independently at each occurrence selected from methyl carbamate, ethyl carbamate, 9-fluorenylmethyl carbamate (Fmoc), 9-(2-sulfo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1,1-dimethyl-2,2-dibromoethyl carbamate (DB-t-BOC), 1,1-dimethyl-2,2,2-trichloroethyl carbamate (TCBOC), benzyl carbamate (Cbz), p-methoxybenzyl carbamate (Moz) and 2,4,6-trimethylbenzyl carbamate, (4-Methoxyphenyl)diphenylmethyl (MMTr); as well as formamide, acetamide, benzamide.

The terms “protecting group for a hydroxyl”, “protecting group for a hydroxyl group”, or “hydroxyl protecting group” as interchangeably used herein, are well known in the art and includes those described in detail in Protecting Groups in Organic Synthesis, T. W. Greene and P. G. M. Wuts, 3^{rd} edition, John Wiley & Sons, New York (1999); Greene's Protective Groups in Organic Synthesis, P. G. M. Wuts, 5^{th} edition, John Wiley & Sons, (2014), and in Current Protocols in Nucleic Acid Chemistry, edited by S. L. Beaucage et al. 06/2012, and hereby in particular in Chapter 2. In a certain embodiment, the “hydroxyl protecting groups” of the present invention include and, typically and preferably are independently at each occurrence selected from, acetyl, benzoyl, benzyl, 3-methoxyethoxymethyl ether (MEM), dimethoxytrityl, [bis-(4-methoxyphenyl)phenylmethyl](DMTr), methoxymethyl ether (MOM), methoxytrityl [(4-methoxyphenyl)diphenylmethyl](MMT), p-methoxybenzyl ether (PMB), methylthiomethyl ether, pivaloyl (Piv), tetrahydropyranyl (THP), tetrahydrofuran (THF), trityl (triphenylmethyl, Tr), silyl ether, such as t-Butyldiphenylsilyl ether (TBDPS), trimethylsilyl (TMS), tert-butyldimethylsilyl (TBDMS), tri-iso-propylsilyloxymethyl (TOM), and triisopropylsilyl (TIPS) ethers; methyl ethers, ethoxyethyl ethers (EE).

Preferred examples of the “hydroxyl protecting groups” of the present invention include and are independently at each occurrence selected from, acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl (TBDMS), t-butyldiphenylsilyl (TBDPS), triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, 4-monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX).

The term “nucleobase”, as used herein, and abbreviated as Bx, refers to unmodified or naturally occurring nucleobases as well as modified or non-naturally occurring nucleobases and synthetic mimetics thereof. A nucleobase is any heterocyclic base that contains one or more atoms or groups of atoms capable of hydrogen bonding to a heterocyclic base of a nucleic acid.

Typical and preferred examples of the nucleobase is a purine base or a pyrimidine base, wherein preferably said purine base is purine or substituted purine, and said pyrimidine base is pyrimidine or substituted pyrimidine. More preferably, the nucleobase is (i) adenine (A), (ii) cytosine (C), (iii) 5-methylcytosine (MeC), (iv) guanine (G), (v) uracil (U), or (vi) 5-methyluracil (MeU), or to a derivative of (i), (ii), (iii), (iv), (v) or (vi).

The terms “derivative of (i), (ii), (iii), (iv), (v) or (vi), and “nucleobase derivative” are used herein interchangeably. Derivatives of (i), (ii), (iii), (iv), (v) or (vi), and nucleobase derivatives, respectively, are known to the skilled person in the art and are described, for example, in Sharma V. K. et al, Med. Chem. Commun., 2014, 5, 1454-1471, and include without limitation 5-hydroxymethyl cytosine, xanthine, hypoxanthine, 2-aminoadenine, alkyl adenine, such as 6-methyl adenine, 2-propyl adenine, alkyl guanine, such as 6-methyl guanine, 2-propyl guanine, 2-thiouracil, 2-thiothymine and 2-thiocytosine, 5-halo uracil, 5-halo cytosine, alkynyl pyrimidine bases, such as 5-propynyl (—C═C—CH₃) uracil, 5-propynyl (—C═C—CH₃) cytosine, 6-azo uracil, 6-azo cytosine, 6-azo thymine, pseudo-uracil, 4-thiouracil; 8-substituted purine bases, such as 8-halo-, 8-amino-, 8-thiol-, 8-thioalkyl-, 8-hydroxyl-adenine or guanine, 5-substituted pyrimidine bases, such as 5-halo-, particularly 5-bromo-, 5-trifluoromethyl-uracil or -cytosine; 7-methylguanine, 7-methyladenine, 2-F-adenine, 2-amino-adenine, 8-azaguanine and 8-azaadenine, 7-deazaguanine, 7-deazaadenine, 3-deazaguanine, 3-deazaadenine, hydrophobic bases, promiscuous bases, size-expanded bases, or fluorinated bases. In certain embodiments, the nucleobase includes without limitation tricyclic pyrimidines, such as 1,3-diazaphenoxazine-2-one, 1,3-diazaphenothiazine-2-one or 9-(2-aminoethoxy)-1,3-diazaphenoxazine-2-one (G-clamp). The term “nucleobase derivative” also includes those in which the purine or pyrimidine base is replaced by other heterocycles, for example 7-deaza-adenine, 7-deazaguanosine, 2-aminopyridine or 2-pyridone. Further nucleobases of the disclosure include without limitation those known to skilled artisan (e.g. U.S. Pat. No. 3,687,808; Swayze et al., The Medicinal Chemistry of Oligonucleotides, in Antisense a Drug Technology, Chapter 6, pp. 143-182 (Crooke, S.T., ed., 2008); The Concise Encyclopedia Of Polymer Science And Engineering, Kroschwitz, J. I., Ed., John Wiley & Sons, 1990, pp. 858-859; Englisch et al., Angewandte Chemie, International Edition, 1991, Vol. 30 (6), pp. 613-623; Sanghvi, Y. S., Antisense Research and Applications, Crooke, S. T. and Lebleu, B., Eds., CRC Press, 1993, pp. 273-302). The term “nucleobase derivative” also includes those in which the purine or pyrimidine base is substituted with a moiety corresponding to the spacer of the present disclosure, in particular, for linking said one or more lipid moiety internally of said oligomeric compound, preferably said oligonucleotide. The specific linkages of said moiety corresponding to the spacer are known to the skilled person in the art. Preferred nucleobase derivatives include methylated adenine, guanine, uracil and cytosine and nucleobase derivatives, preferably of (i), (ii), (iii) or (iv), wherein the respective amino groups, preferably the exocyclic amino groups, are protected by acyl protecting groups or dialkylformamidino, preferably dimethylformamidino (DMF), and further include nucleobase derivatives such as 2-fluorouracil, 2-fluorocytosine, 5-bromouracil, 5-iodouracil, 2,6-diaminopurine, azacytosine and pyrimidine analogs such as pseudoisocytosine and pseudouracil. The preparation of modified nucleobases is known in the art and is described in U.S. Pat. Nos. 3,687,808, 4,845,205; 5,130,302; 5,134,066; 5,175,273; 5,367,066; 5,432,272; 5,457,187; 5,459,255; 5,484,908; 5,502, 177; 5,525,711; 5,552,540; 5,587,469; 5,594, 121; 5,596,091; 5,614,617; 5,645,985; 5,750,692; 5,830,653; 5,763,588; 6,005,096; and 5,681,941.

In some embodiments, the one or more lipid moiety is independently of each other linked to said oligomeric compound at (i) a terminal residue of said oligomeric compound, (ii) the 5′ terminus of said oligomeric compound, (iii) the 3′ terminus of said oligomeric compound; (iv) an internal residue of said oligomeric compound.

The term “terminus” refers to the end or terminus of the oligomeric compound, wherein the integer (3′, 5′, etc.) indicates to the carbon atom of the sugar included in the nucleoside of the oligomeric compound. The term “5′ terminal group” or “3′ terminal group”, as used herein, refers to a group located at the 5′ terminus or 3′ terminus, respectively.

The term “natural” or “naturally occurring”, as interchangeably used herein, refers to compounds that are of natural origin.

The term “exon inclusion” refers to oligonucleotide-mediated processes such as the base-pairing of antisense oligonucleotides to a target pre-mRNA to block an exonic or intronic splicing enhancer and block the corresponding splicing repressor and/or disrupt an unfavorable secondary structure, resulting in more efficient recognition of the exon by the spliceosome and restoration of exon expression.

The term “splicing” is known to the skilled person in the art, and used herein accordingly. The term “splicing”, as used herein, refer to the modification of a pre-mRNA following transcription, in which introns are removed and exons are joined.

The expression “exon skipping”, as used herein, refers to the process leading to the removal from the fully-processed mRNA of an exon which would have been otherwise left in the mature mRNA. By blocking access of spliceosome to one or more splice donor or acceptor sites, or any other site within an exon or intron involved in the definition of splicing, an oligonucleotide can prevent a splicing reaction and cause the exclusion of the targeted exon from a fully-processed mRNA. Exon skipping is achieved in the nucleus during the maturation process of pre-mRNAs. Exon skipping includes the masking of key sequences involved in the splicing of targeted exons by using antisense oligonucleotides that are complementary to such key sequences within a pre-mRNA. For example, the oligomeric compounds provided herein may be suitably employed for exon skipping through the masking of splice sites at intron/exon junctions within a dystrophin pre-mRNA thereby facilitating the deletion of a mutated exon during the processing of the pre-mRNA to a mature mRNA. In the present invention, the oligomeric compound as previously defined are capable of provoking skipping of exon 51 of the human DMD pre-mRNA The expression “provoke skipping of exon 51 of the human DMD pre-mRNA”, as used and described in detail herein, refers to the exclusion of exon 51 allowing the rescue of the DMD mRNA reading-frame (e.g., in cells from patients with appropriate mutations), which can be translated into a truncated semi-functional protein.

The expression “in vitro” refers to an event that takes places outside of a subject's body. In vitro assays encompass cell-based assays in which cells alive or dead are employed and may also encompass a cell-free assay in which no intact cells are employed. On the contrary, the expression “in vivo” refers to an event that takes place in a subject's body.

The expression “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the human subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The term “patient” refers to any subject, afflicted with DMD disease and harboring a large genetic deletion provoking frameshift mutation in the gene coding dystrophin, which could be restored by removing exon 51 during mRNA splicing.

In its broadest meaning, the term “treating” or “treatment” refers to reversing, alleviating, inhibiting the progress of, or preventing the disorder or condition to which such term applies, or one or more symptoms of such disorder or condition.

A “therapeutic effect” as that expression is used herein, encompasses a therapeutic benefit and/or a prophylactic benefit in a human subject. A prophylactic effect includes delaying or eliminating the appearance of a disease or condition, delaying or eliminating the onset of symptoms of a disease or condition, slowing, halting, or reversing the progression of a disease or condition, or any combination thereof.

The expression “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts, preferably said pharmaceutically acceptable salt is the sodium salt.

In case of hydroxyl groups (OH) or thiol groups (SH) typically and preferably bound to P(III) or P(V) and present in said one or more lipid moiety, typically and preferably as part of the group B of said one or more lipid moiety, or present in said spacer, or present in said oligomeric compound, preferably in said oligonucleotide, of the present invention, as part of said internucleosidic linkage group, typically and preferably selected from phosphorothioate or phosphorodiester, each of said hydroxyl groups (OH) or thiol groups (SH) can independently of each other be present as said OH group or in its ionic state such as the O-anion and a pharmaceutically acceptable cation, or as said SH group or in its ionic state such as the S-anion and a pharmaceutically acceptable cation. Further included are any combinations and any states of equilibrium between the aforementioned situations in the inventive compositions, in particular taking further oxygen or sulfur-containing groups on said P(III) or P(V) such as (═O), (═S), another OH or SH group, into account, which is known by the skilled person in the art. For sake of simplicity, in the aspects and embodiments of the present invention, typically only one of the aforementioned situations is described. By way of example, a preferred spacer of the invention is indicated herein as #—NH—C₂₋₂alkylene-OP(O)(SH)-§ . Included herein is, as indicated without limitation, the spacer where the hydrogen is located at the oxygen, thus, #—NH—C₂₋₁alkylene-OP(OH)(S)-§ and all of the pharmaceutically acceptable salt thereof.

Thus, a pharmaceutically acceptable salt in the context of hydroxyl groups (OH) and or thiol groups (SH) typically and preferably bound to P(III) or P(V) and present in said one or more lipid moiety, typically and preferably as part of the group B of said one or more lipid moiety, or present in said spacer, or present in said oligomeric compound, preferably in said oligonucleotide, of the present invention, as part of said internucleosidic linkage group, typically and preferably selected from phosphorothioate or phosphorodiester, refers to the inventive compositions in which one or more of said OH groups or said SH groups are independently of each other be present as said OH group or in its ionic state such as the O-anion and a pharmaceutically acceptable cation thereof, or as said SH group or in its ionic state such as the S-anion and a pharmaceutically acceptable cation, and wherein typically and preferably said pharmaceutically acceptable cation is selected from protonated trimethylamine, protonated diethylamine, protonated methylamine, ammonium, sodium or potassium, further preferably wherein said pharmaceutically acceptable cation is sodium.

By “pharmaceutically acceptable vehicle”, it is meant according to the present invention, any substance which is added to an oligomeric compound according to the present invention to promote its transport, avoid its substantial degradation in said composition and/or increase its half-life. Advantageously, such a pharmaceutically acceptable vehicle is sterile and nonpyrogenic and refers to molecular entities and compositions that do not produce an adverse, allergic or other untoward reaction when administered to a mammal, especially a human, as appropriate. It is chosen depending on the type of application of the pharmaceutical composition of the invention and in particular as a function of its administration mode. Advantageously, a pharmaceutically acceptable vehicle refers to a non-toxic solid, semi-solid or liquid filler, diluent, encapsulating material or formulation auxiliary of any type.

When ranges are used herein to describe, for example, physical or chemical properties such as molecular weight or chemical formulae, all combinations and sub-combinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, from 0% to 10%, from 0% to 5% of the stated number or numerical range.

The antisense technology, in particular Antisense Oligonucleotide (AON)-based approaches started about forty years ago after Zamecnik and Stephenson showed oligonucleotides could be used to downregulate the expression of specific genes. These AONs were originally unmodified synthetic DNA complementary to the targeted mRNA but it rapidly became evident that chemical modifications to protect from nuclease degradation were necessary, especially at the level of phosphodiester internucleotide linkages.

Phosphorothioate (PS) backbones are the most largely used chemical modifications to protect AONs from nuclease activity and increase their stability to target RNA. Typical PS differ from phosphodiester (PO) bonds by the non-bridging phosphate O-atoms being replaced with a S-atom which confers higher stability and increased cellular uptake. PS modifications have demonstrated an elevated efficacy due to an increased bioavailability compared to their PO counterparts and most of the drugs currently under clinical programs include PS bonds. Nevertheless, although their pharmacokinetic advantage, PS modified molecules are known to cause toxicity or undesirable effects mainly due to their capacity to bind plasma proteins. Acute reactions/effects of PS backbones may include immune cell activation, complement activation, that have been particularly reported in monkey studies or prolongation of clotting times that is known to be transient and normalize as oligonucleotides are cleared from blood. To note, low level but sustained complement activation may lead to depletion of complement and damage to the vascular system and kidney.

Another prime site for chemical modification is the 2′-position in the sugar moiety and it has been widely used in the antisense field (e.g., 2′-O-methyl (2′OMe), 2′-O-methoxyethyl (2′OMOE), 2′-fluorinated (2′F) and 2′-O-aminopropyl analogs). Many other structural modifications of the sugar backbone do exist, including phosphorodiamidate morpholino oligomer (PMO), peptide nucleic acid (PNA), locked nucleic acid (LNA), phosphoramidate and methyl-phophonate derivatives as well as tricyclo-DNAs (tc-DNAs). Such an arsenal of AONs has offered many therapeutic options comprising the manipulation of alternative splicing where the antisense molecules are so-called splice switching oligonucleotides (SSO). Here, antisense molecules are used to modulate the ratio of splicing variants or correct splicing defects by inducing exon inclusion or exon skipping; an approach which is suited for the treatment of many neuromuscular disorders including the Duchenne Muscular Dystrophy (DMD).

Duchenne muscular dystrophy (DMD) is an X-linked recessive disorder that affects one in every 3500 live male births. This disorder has an estimated prevalence amongst males of 1-9/100,000 in France, thus qualifying as orphan disease. It is caused by mutations in the DMD gene, which encodes dystrophin, a large protein of 427 kDa found in a variety of tissues, especially in muscle fibers (i.e., striated and smooth muscles) and neurons in particular regions of the central nervous system. Dystrophin is located close to the inner surface of the plasma membrane, connecting the actin cytoskeleton to the extracellular matrix through a membrane dystrophin-associated glycoprotein complex. Lack of dystrophin makes that skeletal muscle fibers are particularly vulnerable to mechanical stress and undergo recurrent cycles of necrosis. As a result, patients display progressive weakness of skeletal muscles, which are with time replaced by adipofibrotic tissue, leading to loss of ambulation by the age of twelve, whereupon premature death is caused by either respiratory failure or cardiomyopathy. In addition, about one third of DMD patients also display cognitive impairment suggesting a noteworthy disruption of neuronal and brain function.

The full-length dystrophin, translated from a major 14-kb mRNA transcript made of 79 exons, is a modular protein that can fortunately support the deletion of multiple exons provided the open reading frame is preserved. This phenomenon occurs in the clinically milder disease Becker Muscular Dystrophy (BMD), where deletions that maintain the open reading frame lead to the synthesis of truncated semi-functional forms of dystrophin. Although DMD is caused by a variety of types of mutations that occur across the gene, most of mutations are large deletions resulting in out-of-frame shortened mRNAs translated into unstable and nonfunctional truncated dystrophins. Hence, it was proposed, twenty years ago, that interfering with the splicing process of elected exons using AONs might be a suitable therapeutic approach for DMD to restore a semi-functional dystrophin thus converting severe DMD into milder BMD.

Two types of compounds have been extensively tested for antisense-induced exon skipping, the 2′-O-methyl-modified ribose oligomers with a full-length phosphorothioate backbone (2′OMe-PS) and the phosphorodiamidate morpholino oligomers (PMO). Both types of antisense molecules have been shown to rescue dystrophin in skeletal muscle after systemic delivery in animal models of DMD and in clinical trials. However, further studies using 2′OMe-PS and PMO AONs targeting exon 51 in DMD patients have failed to show marked clinical benefit, likely due to insufficient levels of dystrophin rescue (Lu et al, Mol. Ther. Nucleic Acids., 2014, vol. 3, e152).

The International application WO 2010/115993 proposes tricyclo-DNA antisense oligonucleotides (tc-DNA AONs) in which all nucleotides are modified by the introduction of a cyclopropane ring in order to restrict conformational flexibility of the backbone. These tc-DNA AONs can be designed for skipping a mutated exon 23 or a mutated exon 51 within a dystrophin pre-mRNA. The tc-DNA AONs designed for skipping a mutated exon 51 are tc-DNA AON H51 (+68+82), tc-DNA AON H51 (+70+84) and tc-DNA AON H51 (+73+87) with the numerical values referring to exon 51 of the human dystrophin gene (DMD gene). Moreover, the International application WO 2013/053928 discloses nucleic acid molecule containing a sequence of tricyclo nucleosides joined by internucleoside phosphorothioate linkages thus forming tricyclo-phosphorothioate DNA molecules (tc-DNA-PS). This application illustrates the use of tc-DNA-PS antisense oligonucleotides for exon 23 skipping of dystrophin pre-mRNA, consolidated by further work in the mdx mouse model of DMD showing that tc-DNA AONs with full PS backbone induced effective skipping of exon 23 to levels 5-6-fold higher than that achieved with 2′OMe-PS and PMO corresponding AONs (Goyenvalle et al, Nat. Med., 2015, vol. 21, pages 270-275). This translated into a greater rescue of dystrophin protein levels, particularly in the diaphragm and heart, where levels reached 50% and 40% respectively, compared to wild-type mice after 12 weeks of treatment. However, the substitution of oxygen by sulfur in the phosphate ester backbone, while significantly improving biodistribution, promotes unspecific protein binding as well as activation of the innate immune system (e.g., complement activation, clotting and elevated proinflammatory cytokines), which may possibly result in the worst in acute toxicity, at best in long-term toxicity (Dirin and Winkler, Expert Opin. Biol. Ther., 2013, vol. 13, pages 875-888; Echevarria et al, Nucleic Acid Ther., 2019, vol. 99, pages 148-160). Alternative schemes, such as controlling phosphorothioate stereochemistry, have been proposed to deal with this situation (Iwamoto et al, Nat. Biotech., 2017, vol. 35, pages 845-851), but recent clinical testing in DMD patients have turned out unsatisfactory.

In addition, the International application WO 2018/193428 proposed another strategy consisting in combining an oligomeric compound comprising one or more tc-DNA nucleosides with one or more lipid moieties covalently linked to this oligomeric compound. Amongst the different oligomeric compounds to which at least one lipid moiety is combined, such as disclosed in this application, the compound SY-0487 has shown the best preliminary results in exon 51 skipping studies and thus is so far considered as the finest tc-DNA-based compound for skipping the exon-51 in DMD. The compound SY-0487 is designed as SYN51 in the present document.

The present invention makes it possible to resolve the technical problems as defined previously and to attain the set aim.

Indeed, without wishing to be bound by any particular theory, a particular sequence in the DMD gene and more particularly in the exon 51 of the pre-mRNA encoded by the human DMD gene has been identified, the reverse complements of which form interesting compounds for treating patients suffering from DMD.

As will be understood by those skilled in the art, in the cell nucleus, eukaryotic genes are transcribed into pre messenger RNAs (pre-mRNA), which contain both exons and introns. To form mature mRNA, splicing occurs at specific sequences at the borders of exons and introns (splice sites) thereby removing introns and connecting exons to one another to form mRNA, which is later translated into protein. During the three last decades, splice-switching approaches have been developed to interfere with such mechanisms in view of DMD treatment. In particular, successful skipping of exon 23 of the mouse DMD gene has been reported in mdx mice by using various types of antisense oligonucleotides annealing the donor splice site at the 3′ end of exon 23. For exon 51, those skilled in the art have developed a number of compounds, some already evaluated at clinical level, selected after screening studies on patient cells, most of them targeting sequences comprised in the region spanning from +66+95 of exon 51 of the pre-mRNA encoded by the human DMD gene.

In the present invention, the particular sequences that were selected are located upstream of the region usually targeted by those skilled in the art; likely because the region covering the sequences implemented in the invention did not appear to be particularly outstanding during in vitro screens as practiced by those skilled in the art.

It has been surprisingly found that the oligomeric compounds of the present invention, in particular when covalently linked to a lipid moiety such as a palmitoyl, has unexpected binding properties for serum proteins. Whatever the species considered, such class of compounds typically binds apolipoproteins (i.e., structural protein components of HDL and LDL), whereas the compounds of the invention preferentially and favorably bind serum albumin in human and non-human primate blood samples; such property potentially markedly improving the bioavailability of the compound as well as its dissemination in skeletal muscles and cardiac tissue. It is noteworthy that this valuable advantage is specific of the oligomeric compounds of the invention.

Thus the present invention includes an oligomeric compound comprising from 10 to 50 monomer subunits, at least part of the sequence of which is complementary to the following sequence: AAGGAAACUGCCAUCUCCAA (SEQ ID NO: 1 in the appended sequence listing).

Advantageously and depending on the monomer subunits they comprise, in some embodiments, the oligomeric compounds of the present invention comprise or consist of oligodeoxyribonucleotides, oligoribonucleotides, morpholinos, tricyclo-DNA oligonucleotides, tricyclo-phosphorothioate-DNA oligonucleotides and LNA oligonucleotides.

It should be noted that in the monomer subunits comprised in the oligomeric compound according to the present invention, one can find not only the five classical nucleobases i.e. adenine (A), cytosine (C), guanine (G), thymine (T), and uracil (U) but also base analogs.

In one aspect, the sequence AAGGAAACUGCCAUCUCCAA (SEQ ID NO: 1 in the appended sequence listing) to which part of the sequence of the oligomeric compound according to the present invention is complementary is the region defined by positions +45+64 of exon 51 of the pre-mRNA encoded by the human DMD gene. The exon 51 of the pre-mRNA encoded by the human DMD gene is of sequence:

(SEQ ID NO: 2 in the appended sequence listing)
CUCCUACUCAGACUGUUACUCUGGUGACACAACCUGUGGUUACUAAGGA
AACUGCCAUCUCCAAACUAGAAAUGCCAUCUUCCUUGAUGUUGGAGGUA
CCUGCUCUGGCAGAUUUCAACCGGGCUUGGACAGAACUUACCGACUGGC
UUUCUCUGCUUGAUCAAGUUAUAAAAUCACAGAGGGUGAUGGUGGGUGA
CCUUGAGGAUAUCAACGAGAUGAUCAUCAAGCAGAAG.

Advantageously, in some embodiments, at least part of the sequence of the oligomeric compound according to the present invention is complementary to the sequence corresponding to the region +48+62 of SEQ ID NO: 2 in the appended sequence listing. This region also corresponds to the region +4+18 of SEQ ID NO: 1 in the appended sequence listing.

Thus, the oligomeric compound of the present invention and the target nucleotide sequence of the pre-mRNA are complementary to each other when a sufficient number of corresponding positions in each molecule are occupied by nucleobases that can bond with each other to allow stable association between the oligomeric compound of the present invention and the target nucleotide sequence of the pre-mRNA as indicated above. One skilled in the art recognizes that the inclusion of mismatches is possible without eliminating the ability of the oligomeric compounds to remain in association. Therefore, described herein are oligomeric compounds of the present invention, advantageously being antisense oligonucleotides, that may comprise up to about 20% nucleotides that are mismatched (i.e., are not nucleobase complementary to the corresponding nucleotides of the target). In particular the oligomeric compounds of the present invention, advantageously being antisense oligonucleotides, contain no more than about 15%, more preferably not more than about 10%, most preferably not more than 5% or no mismatches.

Typically, the oligomeric compound according to the present invention comprises or consists of an antisense oligonucleotide.

In addition, in the present invention, the antisense oligonucleotide (AON) sequences are selected so as to be specific, i.e. the AON's are fully complementary only to the sequences of the targeted pre-mRNA and not to other nucleic acid sequences. The AONs used in the practice of the invention may be of any suitable type (e.g., oligodeoxyribonucleotides, oligoribonucleotides, morpholinos, tricyclo-DNA, tricyclo-phosphorothioate-DNA, LNA, U7— or U1-modified AONs or conjugate products thereof such as peptide-conjugated or nanoparticle-complexed AONs), which are known to the skilled person in the art (Bell et al, ChemBioChem, 2009, vol. 10, pages 2691-2703). Oligomeric compounds and in particular AONs according to the invention are generally from about 10 to about 50 nucleotides in length, in particular from about 11 to about 40 nucleotides, from about 12 to about 30 nucleotides or from about 13 to about 20 nucleotides, and may be for example, about 10, or about 15, or about 20 or about 30 nucleotides or more in length. Typically, morpholino-AONs are about 25-30 nucleotides long, PPMO AONs are about 20-25 nucleotides long, and tricyclo-AONs are about 10-20 nucleotides long, U7 and U1-modified AONs may possibly carry longer antisense sequences of about 50 nucleotides. The expression “about X nucleotides” means X nucleotides ±2 nucleotides.

In a particular embodiment, the oligomeric compound according to the present invention comprises at least one nucleotide sequence having at least 70% identity with the reverse complement of SEQ ID NO: 1.

Thus, the oligomeric compound according to the present invention comprises at least one nucleotide sequence having at least 70% identity and may exhibit at least 75%, at least 80%, at least 85%, at least 90%, at least 95% or even at least 99% identity with the reverse complement of SEQ ID NO: 1.

In a more particular embodiment, the oligomeric compound according to the present invention comprises at least one nucleotide sequence having at least 70% identity with the following tc-DNA nucleotide sequence:

GGAGATGGCAGTTTC (SEQ ID NO: 3 in the appended sequence listing).

Thus, the oligomeric compound according to the present invention comprises at least one nucleotide sequence having at least 70% identity and may exhibit at least 73%, at least 75%, at least 80%, at least 85%, at least 90%, at least 93%, at least 95%, or even at least 99% identity with the tc-DNA nucleotide sequence SEQ ID NO: 3.

The identity percent is statistic and the differences between both sequences are randomly distributed along these sequences. The differences between both sequences can consist of different modification types of the sequences: deletions, substitutions or additions of nucleotide (or amino acid) residues.

In a particular embodiment, the oligomeric compound according to the present invention comprises at least one nucleotide sequence identical to the tc-DNA nucleotide sequence SEQ ID NO: 3.

For use in vivo, the oligomeric compounds and in particular AONs according to the invention may be stabilized. A “stabilized” oligomeric compound or AON refers to an oligomeric compound or AON that is relatively resistant to in vivo degradation (e.g., via an exo- or endo-nuclease). Stabilization can be a function of length or secondary structure. Alternatively, oligomeric compound or AON stabilization can be accomplished via phosphate backbone modifications.

Preferred stabilized oligomeric compounds or AONs of the instant invention have a modified backbone, e.g., have phosphorothioate linkages to provide maximal activity and protect the oligomeric compound or AON from degradation by intracellular exo- and endo-nucleases. Other possible stabilizing modifications include phosphodiester modifications, combinations of phosphodiester and phosphorothioate modifications, methyl-phosphonate, methyl-phosphorothioate, phosphorodithioate, p-ethoxy, and combinations thereof. Chemically stabilized, modified versions of the oligomeric compounds or AONs also include “Morpholinos” (phosphorodiamidate morpholino oligomers, PMOs), 2′-O-Met oligomers, tricyclo-DNA (tc-DNA) oligomers (International application WO 2010/115993), tricyclo-phosphorothioate DNA oligomers (International application WO 2013/053928), LNAs etc., which are all known to the skilled person in the art (Bell et al, ChemBioChem, 2009, vol. 10, pages 2691-2703).

In a particular embodiment of the present invention, the oligomeric compound comprises mainly tricyclo-deoxyribonucleic acid (tc-DNA) nucleosides. Consequently, the oligomeric compound of the invention comprises or consists of a tricyclo-DNA antisense oligonucleotide. In this embodiment, the tricyclo-DNA antisense oligonucleotide according to the present invention comprises or consists of a nucleotide sequence corresponding to the nucleotide sequence SEQ ID NO: 3.

In this embodiment, the different tc-DNA nucleosides may be joined by phosphodiester linkages. Alternatively, at least two adjacent tc-DNA nucleosides may be joined by a phosphorothioate (PS) linkage. The expressions “phosphorothioate linkage” or “phosphorothioate modification”, as interchangeably used herein, refers to a “5′ . . . —O—P(S)—O— . . . 3” moiety between two adjacent nucleosides in a nucleic acid molecule. Advantageously all the tc-DNA nucleosides in the oligomeric compound according to the invention are joined by PS linkages. Thus the oligomeric compound of the invention comprises or consists of a tricyclo-phosphorothiate DNA antisense oligonucleotide. If other modifications are present in the oligomeric compound of the disclosure, the latter include phosphodiester, methylphosphonate, methyl-phosphorothioate, phosphorodithioate, and p-ethoxy modifications, and combinations thereof.

In an embodiment, the tc-DNA nucleosides of the oligomeric compounds comprise a compound of Formula (1):

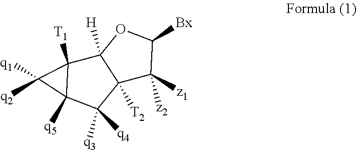

wherein:
Bx is a nucleobase;
one of T₁ and T₂ is an internucleosidic linkage group, and the other of Ti and T₂ is OR₁, OR₂, a 5′ terminal group, a 3′ terminal group or an internucleosidic linkage group, wherein R₁ is H or a hydroxyl protecting group, and R₂ is a phosphorus moiety;
q₁, q₂, q₃, q₄ and q₅ are each independently selected from the group consisting of hydrogen (H), halogen, C₁₋₆alkyl, C₂₋₆alkenyl, C₂₋₆alkynyl, substituted C₁₋₆alkyl, substituted C₂₋₆alkenyl, substituted C₂₋₆alkynyl, and —(CH₂)ₙ—C(O)—R₆′, wherein n is 0 to 6 and wherein R_{6′} is selected from the group consisting of OH, NH₂, O—C₁₋₃₂1kyl and NH—C₁₋₃₂alkyl;
z₁ and z₂ are each independently selected from the group consisting of H, halogen, C₁₋₆alkyl, C₁₋₆alkoxyl, O—C₂₋₆alkenyl, O—C₂₋₆alkynyl, substituted C₁₋₆alkyl, substituted C₁₋₆alkoxy, substituted O—C₂₋₆alkenyl, and substituted O—C₂₋₆alkynyl;
or a pharmaceutically-acceptable salt thereof.

In an embodiment, the tc-DNA nucleosides of the oligomeric compounds comprise a compound of Formula (1), wherein q₅ is H.

In an embodiment, the tc-DNA nucleosides of the oligomeric compounds comprise a compound of Formula (1), wherein Bx is selected from the group consisting of thymine, adenine, guanine, and cytosine. In an embodiment, the tc-DNA nucleosides of the oligomeric compounds comprise a compound of Formula (1), wherein Bx is a modified base. In an embodiment, the tc-DNA nucleosides of the oligomeric compounds of the invention comprise a compound of Formula (1), wherein Bx is a modified base selected from the group consisting of 5-methylcytosine, 5-bromouracil, inosine, and 2,6-diaminopurine.

In an embodiment, the tc-DNA nucleosides of the oligomeric compounds comprise a compound of Formula (2):

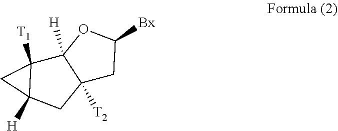

wherein:
Bx is a nucleobase;
one of T₁ and T₂ is an internucleosidic linkage group, and the other of T₁ and T₂ is OR₁, OR₂, a 5′ terminal group, a 3′ terminal group or a internucleosidic linkage group, wherein R₁ is H or a hydroxyl protecting group, and R₂ is a phosphorus moiety.

In an embodiment, the tc-DNA nucleosides of the oligomeric compounds comprise a compound of Formula (2), wherein Bx is selected from the group consisting of thymine, adenine, guanine, and cytosine. In an embodiment, the tc-DNA nucleosides of the oligomeric compounds of the invention comprise a compound of Formula (2), wherein Bx is a modified base. In an embodiment, the tc-DNA nucleosides of the oligomeric compounds of the invention comprise a compound of Formula (2), wherein Bx is a modified base selected from the group consisting of 5-methylcytosine, 5-bromouracil, inosine, and 2,6-diaminopurine.

In an embodiment, the tc-DNA nucleosides of the oligomeric compounds comprise a compound of Formula (3) (also known as a C(6′)-functionalized tc-DNA):

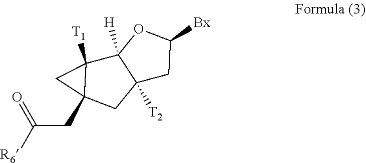

wherein:
Bx is a nucleobase;
R_{6′} is selected from the group consisting of OH, NH₂, O—C₁₋₃₂alkyl and NH—C₁₋₃₂alkyl;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the tc-DNA nucleosides of the oligomeric compounds comprise a compound of Formula (3), wherein Bx is selected from the group consisting of thymine, adenine, guanine, and cytosine. In an embodiment, the tc-DNA nucleosides of the oligomeric compounds of the invention comprise a compound of Formula (3), wherein Bx is a modified base. In an embodiment, the tc-DNA nucleosides of the oligomeric compounds of the invention comprise a compound of Formula (3), wherein Bx is a modified base selected from the group consisting of 5-methylcytosine, 5-bromouracil, inosine, and 2,6-diaminopurine.

In an embodiment, the tc-DNA nucleosides of the oligomeric compounds comprise a compound of Formula (4) (also known as 6′-fluoro-tc-DNA):

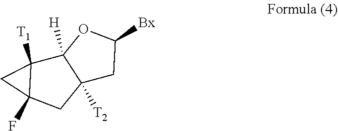

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the tc-DNA nucleosides of the oligomeric compounds comprise a compound of Formula (4), wherein Bx is selected from the group consisting of thymine, adenine, guanine, and cytosine. In an embodiment, the tc-DNA nucleosides of the oligomeric compounds of the invention comprise a compound of Formula (4), wherein Bx is a modified base. In an embodiment, the tc-DNA nucleosides of the oligomeric compounds of the invention comprise a compound of Formula (4), wherein Bx is a modified base selected from the group consisting of 5-methylcytosine, 5-bromouracil, inosine, and 2,6-diaminopurine.

In an embodiment, the tc-DNA nucleosides of the oligomeric compounds comprise a compound of Formula (5) (also known as 2′-fluoro-tc-DNA):

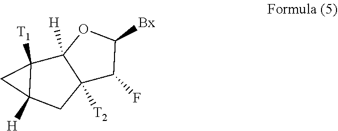

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the tc-DNA nucleosides of the oligomeric compounds comprise a compound of Formula (5), wherein Bx is selected from the group consisting of thymine, adenine, guanine, and cytosine. In an embodiment, the tc-DNA nucleosides of the oligomeric compounds of the invention comprise a compound of Formula (5), wherein Bx is a modified base. In an embodiment, the tc-DNA nucleosides of the oligomeric compounds of the invention comprise a compound of Formula (5), wherein Bx is a modified base selected from the group consisting of 5-methylcytosine, 5-bromouracil, inosine, and 2,6-diaminopurine.

Thus, in an embodiment, said one or more nucleosides tc-DNA nucleosides of the oligomeric compounds comprise a compound of Formula (5′) (also known as 2′-fluoro-tc-ANA):

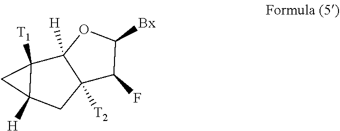

wherein:
Bx is a nucleobase;
one of T₁ and T₂ is an internucleosidic linkage group, and the other of Ti and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the tc-DNA nucleosides of the oligomeric compounds comprise a compound of Formula (5′), wherein Bx is selected from the group consisting of thymine, adenine, guanine, and cytosine. In an embodiment, the tc-DNA nucleosides of the oligomeric compounds of the invention comprise a compound of Formula (5′), wherein Bx is a modified base. In an embodiment, the tc-DNA nucleosides of the oligomeric compounds of the invention comprise a compound of Formula (5′), wherein Bx is a modified base selected from the group consisting of 5-methylcytosine, 5-bromouracil, inosine, and 2,6-diaminopurine.

General methods of preparation of compounds of Formula (1) and Formula (2) for use with oligomeric compounds are known in the art, including the methods described in U.S. patent applications Nos. 2015/0141637, 2016/0002280, and 2014/0296323, the disclosures of which are incorporated by reference herein. Standard phosphoramidite building blocks for tc-DNA have been described in the art, e.g., in Steffens and Leumann, Helv. Chim. Acta 1997, 80, 2426-2439. Methods of preparing compounds of Formula (3) have been described, e.g., in Lietard and Leumann, J. Org. Chem. 2012, 77, 4566-77, the disclosure of which is incorporated by reference herein. Methods of preparing compounds of Formula (4) have been described, e.g., in Medvecky, Istrate, and Leumann, J. Org. Chem. 2015, 80, 3556-65, the disclosure of which is incorporated by reference herein. Methods of preparing compounds of Formula (5) and (5′) have been described, e.g., in Istrate, Medvecky, and Leumann, Org. Lett. 2015, 17, 1950-53, the disclosure of which is incorporated by reference herein.

In another particular embodiment of the present invention, the oligomeric compound comprises at least one tricyclo-deoxyribonucleic acid (tc-DNA) nucleoside and at least one modified ribonucleic acid nucleoside. Any modified RNA nucleoside known to those skilled in the art can be implemented in the present invention. Modified RNA nucleosides confer flexibility to the oligomeric molecule in which they are introduced. In particular, this modified RNA nucleoside is a 2′-modified RNA nucleoside such as 2′-O-methyl, 2′-methoxyethoxy, 2′-fluoro, 2′-allyl, 2′-O-[2-(methylamino)-2-oxoethyl], 2′-amino and 2′-O—(N-methylcarbamate). More particularly, this modified RNA nucleoside is a 2′-O-methyl RNA nucleoside. In addition, the monomer subunits of the oligomeric compound according to this particular embodiment are typically joined by phosphodiester internucleoside linkages.

2′-Modified RNA Nucleosides and other Nucleosides

In an embodiment, the one or more nucleosides other than tc-DNA nucleosides of the oligomeric compounds are independently of each other 2′-modified ribonucleic acid (2′-modified-RNA) nucleosides.

In an embodiment, the one or more nucleosides other than tc-DNA nucleosides of the oligomeric compounds is an RNA nucleoside of Formula (6) (a RNA nucleoside):

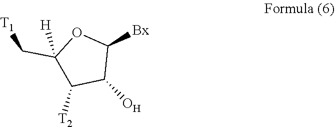

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the one or more nucleosides other than tc-DNA nucleosides of the oligomeric compounds is an RNA nucleoside of Formula (6), wherein Bx is selected from the group consisting of cytosine, adenine, guanine, and uracil. In an embodiment, the 2′-modified-RNA nucleosides of the oligomeric compounds comprise a compound of Formula (6), wherein Bx is a modified base. In an embodiment, the 2′-modified-RNA nucleosides of the oligomeric compounds comprise a compound of Formula (6), wherein Bx is a modified base selected from the group consisting of 5-methylcytosine, 5-methyluracil, 5-bromouracil, inosine, and 2,6-diaminopurine.

In an embodiment, the 2′-modified-RNA nucleosides of the oligomeric compounds of the preferred inventive compositions comprise a compound of Formula (7) (a 2′-O-methyl-RNA nucleoside):

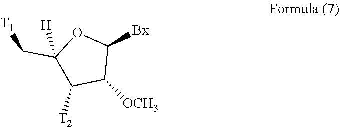

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the 2′-modified-RNA nucleosides of the oligomeric compounds comprise a compound of Formula (7), wherein Bx is selected from the group consisting of cytosine, adenine, guanine, and uracil. In an embodiment, the 2′-modified-RNA nucleosides of the oligomeric compounds comprise a compound of Formula (7), wherein Bx is a modified base. In an embodiment, the 2′-modified-RNA nucleosides of the oligomeric compounds comprise a compound of Formula (7), wherein Bx is a modified base selected from the group consisting of 5-methylcytosine, 5-methyluracil, 5-bromouracil, inosine, and 2,6-diaminopurine.

In an embodiment, the 2′-modified-RNA nucleosides of the oligomeric compounds comprise a compound of Formula (8) (a 2′-O-propargyl-RNA nucleoside):

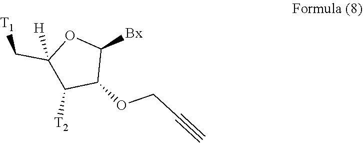

wherein:
Bx is a nucleobase;
one of T₁ and T₂ is an internucleosidic linkage group, and the other of T₁ and T₂ is OR₁, OR₂, a 5′ terminal group, a 3′ terminal group or a internucleosidic linkage group, wherein R₁ is H or a hydroxyl protecting group, and R₂ is a phosphorus moiety.

In an embodiment, the 2′-modified-RNA nucleosides of the oligomeric compounds comprise a compound of Formula (8), wherein Bx is selected from the group consisting of cytosine, adenine, guanine, and uracil. In an embodiment, the 2′-modified-RNA nucleosides of the oligomeric compounds comprise a compound of Formula (8), wherein Bx is a modified base. In an embodiment, the 2′-modified-RNA nucleosides of the oligomeric compounds comprise a compound of Formula (8), wherein Bx is a modified base selected from the group consisting of 5-methylcytosine, 5-methyluracil, 5-bromouracil, inosine, and 2,6-diaminopurine.

In an embodiment, the 2′-modified-RNA nucleosides of the oligomeric compounds comprise a compound of Formula (9) (a 2′-O-propylamino-RNA nucleoside):

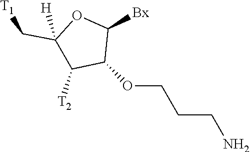

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the 2′-modified-RNA nucleosides of the oligomeric compounds comprise a compound of Formula (9), wherein Bx is selected from the group consisting of cytosine, adenine, guanine, and uracil. In an embodiment, the 2′-modified-RNA nucleosides of the oligomeric compounds comprise a compound of Formula (9), wherein Bx is a modified base. In an embodiment, the 2′-modified-RNA nucleosides of the oligomeric compounds comprise a compound of Formula (9), wherein Bx is a modified base selected from the group consisting of 5-methylcytosine, 5-methyluracil, 5-bromouracil, inosine, and 2,6-diaminopurine.

In an embodiment, the 2′-modified-RNA nucleosides of the oligomeric compounds comprise a compound of Formula (10) (a 2′-amino-RNA nucleoside):

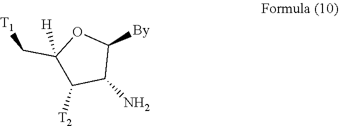

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the 2′-modified-RNA nucleosides of the oligomeric compounds comprise a compound of Formula (10), wherein Bx is selected from the group consisting of cytosine, adenine, guanine, and uracil. In an embodiment, the 2′-modified-RNA nucleosides of the oligomeric compounds comprise a compound of Formula (10), wherein Bx is a modified base. In an embodiment, the 2′-modified-RNA nucleosides of the oligomeric compounds comprise a compound of Formula (10), wherein Bx is a modified base selected from the group consisting of 5-methylcytosine, 5-methyluracil, 5-bromouracil, inosine, and 2,6-diaminopurine.

In an embodiment, the 2′-modified-RNA nucleosides of the oligomeric compounds comprise a compound of Formula (11) (a 2′-fluoro-RNA nucleoside):

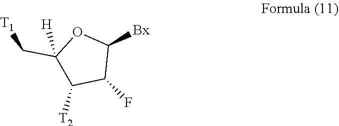

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the 2′-modified-RNA nucleosides of the oligomeric compounds comprise a compound of Formula (11), wherein Bx is selected from the group consisting of cytosine, adenine, guanine, and uracil. In an embodiment, the 2′-modified-RNA nucleosides of the oligomeric compounds comprise a compound of Formula (11), wherein Bx is a modified base. In an embodiment, the 2′-modified-RNA nucleosides of the oligomeric compounds comprise a compound of Formula (11), wherein Bx is a modified base selected from the group consisting of 5-methylcytosine, 5-methyluracil, 5-bromouracil, inosine, and 2,6-diaminopurine.

In an embodiment, the one or more nucleosides other than tc-DNA nucleosides of the oligomeric compounds comprise a compound of Formula (11′) (a 2′-deoxy 2′-fluoro-arabino nucleoside (2′-FANA):

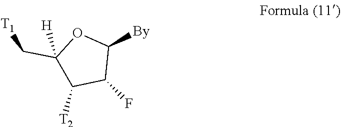

wherein:
Bx is a nucleobase;
one of T₁ and T₂ is an internucleosidic linkage group, and the other of Ti and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the one or more nucleosides other than tc-DNA nucleosides of the oligomeric compounds comprise a compound of Formula (11′), wherein Bx is selected from the group consisting of cytosine, adenine, guanine, and uracil. In an embodiment, the 2′-modified-RNA nucleosides of the oligomeric compounds comprise a compound of Formula (11′), wherein Bx is a modified base. In an embodiment, the 2′-modified-RNA nucleosides of the oligomeric compounds comprise a compound of Formula (11′), wherein Bx is a modified base selected from the group consisting of 5-methylcytosine, 5-methyluracil, 5-bromouracil, inosine, and 2,6-diaminopurine.

In an embodiment, the 2′-modified-RNA nucleosides of the oligomeric compounds comprise a compound of Formula (12) (a 2′-O-methoxyethyl-RNA, or 2′-MOE, nucleoside):

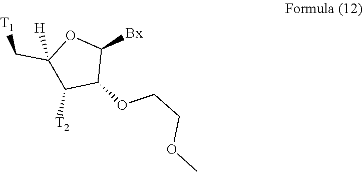

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the 2′-modified-RNA nucleosides of the oligomeric compounds comprise a compound of Formula (12), wherein Bx is selected from the group consisting of cytosine, adenine, guanine, and uracil. In an embodiment, the 2′-modified-RNA nucleosides of the oligomeric compounds comprise a compound of Formula (12), wherein Bx is a modified base. In an embodiment, the 2′-modified-RNA nucleosides of the oligomeric compounds comprise a compound of Formula (12), wherein Bx is a modified base selected from the group consisting of 5-methylcytosine, 5-methyluracil, 5-bromouracil, inosine, and 2,6-diaminopurine.

In an embodiment, the one or more nucleosides other than tc-DNA nucleosides of the oligomeric compounds comprise a compound of Formula (13) (a morpholino nucleoside):

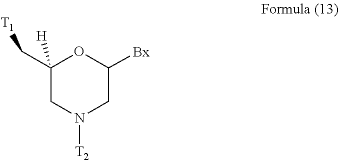

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the one or more nucleosides other than tc-DNA nucleosides of the oligomeric compounds comprise a compound of Formula (13), wherein Bx is selected from the group consisting of cytosine, adenine, guanine, and uracil. In an embodiment, the 2′-modified-RNA nucleosides of the oligomeric compounds comprise a compound of Formula (13), wherein Bx is a modified base. In an embodiment, the 2′-modified-RNA nucleosides of the oligomeric compounds comprise a compound of Formula (13), wherein Bx is a modified base selected from the group consisting of 5-methylcytosine, 5-methyluracil, 5-bromouracil, inosine, and 2,6-diaminopurine.

In an embodiment, the 2′-modified-RNA nucleosides of the oligomeric compounds comprise a compound of Formula (14) (a locked nucleic acid or LNA nucleoside):

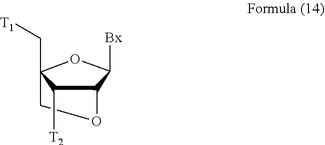

wherein:
Bx is a nucleobase;
one of T₁ and T₂ is an internucleosidic linkage group, and the other of T₁ and T₂ is OR₁, OR₂, a 5′ terminal group, a 3′ terminal group or an internucleosidic linkage group, wherein R₁ is H or a hydroxyl protecting group, and R₂ is a phosphorus moiety.

In an embodiment, the 2′-modified-RNA nucleosides of the oligomeric compounds comprise a compound of Formula (14), wherein Bx is selected from the group consisting of cytosine, adenine, guanine, and uracil. In an embodiment, the 2′-modified-RNA nucleosides of the oligomeric compounds comprise a compound of Formula (14), wherein Bx is a modified base. In an embodiment, the 2′-modified-RNA nucleosides of the oligomeric compounds comprise a compound of Formula (14), wherein Bx is a modified base selected from the group consisting of 5-methylcytosine, 5-methyluracil, 5-bromouracil, inosine, and 2,6-diaminopurine.

General methods of preparation of compounds of Formula (6) to Formula (14) for use with oligomeric compounds are known in the art, including the methods described in U.S. patents Nos. 4,981,957; 5, 118,800; 5,319,080; 5,359,044; 5,393,878; 5,446, 137; 5,466, 786; 5,514,785; 5,519,134; 5,567,811;5,576,427; 5,591,722; 5,597,909; 5,610,300; 5,627,053; 5,639,873; 5,646,265; 5,670,633; 5,700,920; 5,792,847; and 6,600,032; U.S. patent application Nos. 2015/0141637, 2016/0002280, and 2014/0296323; and Renneberg, et al, J. Am. Chem. Soc., 2002, 124, 5993-6002, the disclosures of which are incorporated by reference herein.

In an embodiment, said oligomeric compound further comprises one or more nucleosides other than tc-DNA nucleosides, wherein said one or more nucleosides other than tc-DNA nucleosides are independently of each other selected from
ribonucleic acid (RNA) nucleosides;
deoxyribonucleic acid (DNA) nucleosides;
2′-modified-RNA nucleosides
bicyclic nucleic acid (2′,4′-BNA) nucleosides, preferably selected from 2′,4′-BNA having a 2′-O—N—C bridged system (2′,4′-BNA^{NC}), stereoisomer of LNA - [symbol] -L-LNA and Ethylene nucleic acid (ENA) nucleosides;
peptide nucleic acids (PNAs) nucleosides;
2′-deoxy 2′-fluoro-arabino (FANA) nucleosides;
hexitol nucleic acids (HNAs) nucleosides; and
phosphorodiamidate morpholino (PMO) nucleosides.

Further nucleosides useful for the present invention are known for the skilled person in the art such as other lipophilic 2′-O-alkyl RNA as described in Biochemistry, 2005, 44, 9045-9057.

In an embodiment, the oligomeric compounds comprise non-nucleosides, also known in the art as non-nucleoside linkers, non-nucleotide linkers, and non-nucleotidylic linkers, which are highly flexible substitutes for the sugar carbons of, e.g., a ribofuranone moiety, and which can be used to replace the tc-DNA nucleosides and the nucleosides other than the tc-DNA nucleosides of the present oligomeric compounds. An exemplary non-nucleotide is the 1,3-propanediol group shown in Formula (15), which is shown joining two exemplary phosphorodiester internucleosidic linkages:

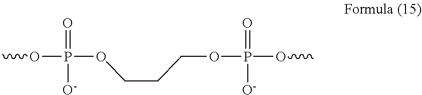

The wavy lines in Formula (15) signify additional oligomeric repeating nucleoside and internucleosidic linkages units as described herein.

The non-nucleotides of the present invention may be used with any of the internucleosidic linkages described herein, including embodiments wherein the phosphorodiester internucleosidic linkages shown in Formula (15) are replaced with one or more phosphorothioate internucleosidic linkages.

In an embodiment, a non-nucleotide is a 1,3-propanediol group. The synthesis and incorporation of 1,3-propanediol groups into oligomeric compounds is known in the art and is described, e.g., in Seela and Kaiser, Nuc. Acids Res. 1987, 15, 3113-29. In an embodiment, the oligomeric compounds include 1, 2, 3, 4, 5, 6, 7, 8, 9, 10, 11, or 12 1,3-propanediol groups linked by phosphorothioate internucleosidic linkages, phosphorodiester internucleosidic linkages, or mixtures thereof.

Alternative non-nucleosides may also be used with the oligomeric compounds of the present invention, such as ethylene glycol oligomers of various lengths (i.e., one, two, three, or more ethylene glycol units joined to form a single non-nucleoside). Various suitable ethylene glycol groups are described, e.g., in Pils and Micura, Nuc. Acids Res. 2000, 28, 1859-63. The synthesis and use of non-nucleosides have also been described in, e.g., U.S. Pat. No. 5,573,906, the disclosure of which is incorporated by reference herein.

In some embodiments, said oligomeric compound does not comprise nucleosides other than tc-DNA nucleosides.

In some embodiments, said oligomeric compound further comprises one or more nucleosides other than tc-DNA nucleosides.

In some embodiments, said oligomeric compound comprises one or more tc-DNA nucleosides and one or more nucleosides other than tc-DNA nucleosides, wherein 50% or more of all nucleosides are tc-DNA nucleosides.

In some embodiments, said oligomeric compound comprises one or more tc-DNA nucleosides and one or more nucleosides other than tc-DNA nucleosides, wherein 60% or more of all nucleosides are tc-DNA nucleosides.

In some embodiments, said oligomeric compound comprises one or more tc-DNA nucleosides and one or more nucleosides other than tc-DNA nucleosides, wherein 70% or more of all nucleosides are tc-DNA nucleosides.

In some embodiments, said oligomeric compound comprises one or more tc-DNA nucleosides and one or more nucleosides other than tc-DNA nucleosides, wherein 75% or more of all nucleosides are tc-DNA nucleosides.

In some embodiments, said oligomeric compound comprises one or more tc-DNA nucleosides and one or more nucleosides other than tc-DNA nucleosides, wherein 80% or more of all nucleosides are tc-DNA nucleosides.

In some embodiments, said oligomeric compound comprises one or more tc-DNA nucleosides and one or more nucleosides other than tc-DNA nucleosides, wherein 85% or more of all nucleosides are tc-DNA nucleosides.

In some embodiments, said oligomeric compound comprises one or more tc-DNA nucleosides and one or more nucleosides other than tc-DNA nucleosides, wherein 90% or more of all nucleosides are tc-DNA nucleosides.

In some embodiments, said oligomeric compound comprises one or more tc-DNA nucleosides and one or more nucleosides other than tc-DNA nucleosides, wherein 95% or more of all nucleosides are tc-DNA nucleosides.

In some embodiments, said oligomeric compound further comprises one or more nucleosides other than tc-DNA nucleosides, wherein said one or more nucleosides other than tc-DNA nucleosides are independently of each other selected from
i. 2′-modified ribonucleic acid (2′-modified-RNA) nucleosides;
ii. ribonucleic acid (RNA) nucleosides;
iii. deoxyribonucleic acid (DNA) nucleosides;
iv. locked nucleic acid (LNA) nucleosides;
v. peptide nucleic acids (PNAs) nucleosides;
vi. 2′-deoxy 2′-fluoro-arabino nucleosides;
vii. hexitol nucleic acids (HNAs) nucleosides; and
viii. phosphorodiamidate morpholino (PMO) nucleosides.

In some embodiments, said oligomeric compound further comprises one or more nucleosides other than tc-DNA nucleosides, wherein said one or more nucleosides other than tc-DNA nucleosides are independently of each other 2′-modified ribonucleic acid (2′-modified-RNA) nucleosides.

In some embodiments, said 2′-modified-RNA nucleosides are incorporated in at least two adjacent positions that form self-complementary Watson-Crick base pairs.

In some embodiments, said 2′-modified-RNA nucleosides are incorporated at three or more adjacent positions that form self-complementary Watson-Crick base pairs.

In some embodiments, said oligomeric compound further comprises one or more nucleosides other than tc-DNA nucleosides, wherein said one or more nucleosides other than tc-DNA nucleosides are independently of each other ribonucleic acid (RNA) nucleosides.

In some embodiments, said oligomeric compound further comprises one or more nucleosides other than tc-DNA nucleosides, wherein said one or more nucleosides other than tc-DNA nucleosides are independently of each other deoxyribonucleic acid (DNA) nucleosides.

In some embodiments, said oligomeric compound further comprises one or more nucleosides other than tc-DNA nucleosides, wherein said one or more nucleosides other than tc-DNA nucleosides are independently of each other locked nucleic acid (LNA) nucleosides.

In some embodiments, said oligomeric compound further comprises one or more nucleosides other than tc-DNA nucleosides, wherein said one or more nucleosides other than tc-DNA nucleosides are independently of each other peptide nucleic acids (PNAs) nucleosides.

In some embodiments, said oligomeric compound further comprises one or more nucleosides other than tc-DNA nucleosides, wherein said one or more nucleosides other than tc-DNA nucleosides are independently of each other 2′-deoxy 2′-fluoro-arabino nucleosides.

In some embodiments, said oligomeric compound further comprises one or more nucleosides other than tc-DNA nucleosides, wherein said one or more nucleosides other than tc-DNA nucleosides are independently of each other hexitol nucleic acids (HNAs) nucleosides.

In some embodiments, said oligomeric compound further comprises one or more nucleosides other than tc-DNA nucleosides, wherein said one or more nucleosides other than tc-DNA nucleosides are independently of each other phosphorodiamidate morpholino (PMO) nucleosides.

In some embodiments, said oligomeric compound further comprises one or more nucleosides other than tc-DNA nucleosides, wherein said one or more nucleosides other than tc-DNA nucleosides are independently of each other selected from
i. RNA nucleosides;
ii. 2′-O-methyl-RNA nucleosides;
iii. 2′-O-propargyl-RNA nucleosides;
iv. 2′-O-propylamino-RNA nucleosides;
v. 2′-O-amino-RNA nucleosides;
vi. 2′-fluoro-RNA nucleosides;
vii. 2′-O-methoxyethyl-RNA nucleosides;
viii. morpholino nucleosides; and
ix. locked nucleic acid nucleosides.

In some embodiments, said oligomeric compound further comprises one or more nucleosides other than tc-DNA nucleosides, wherein said one or more nucleosides other than tc-DNA nucleosides are independently of each other RNA nucleosides.

In some embodiments, said oligomeric compound further comprises one or more nucleosides other than tc-DNA nucleosides, wherein said one or more nucleosides other than tc-DNA nucleosides are independently of each other 2′-O-methyl-RNA nucleosides.

In some embodiments, said oligomeric compound further comprises one or more nucleosides other than tc-DNA nucleosides, wherein said one or more nucleosides other than tc-DNA nucleosides are independently of each other 2′-O-propargyl-RNA nucleosides.

In some embodiments, said oligomeric compound further comprises one or more nucleosides other than tc-DNA nucleosides, wherein said one or more nucleosides other than tc-DNA nucleosides are independently of each other 2′-O-propylamino-RNA nucleosides.

In some embodiments, said oligomeric compound further comprises one or more nucleosides other than tc-DNA nucleosides, wherein said one or more nucleosides other than tc-DNA nucleosides are independently of each other 2′-O-amino-RNA nucleosides.

In some embodiments, said oligomeric compound further comprises one or more nucleosides other than tc-DNA nucleosides, wherein said one or more nucleosides other than tc-DNA nucleosides are independently of each other 2′-fluoro-RNA nucleosides.

In some embodiments, said oligomeric compound further comprises one or more nucleosides other than tc-DNA nucleosides, wherein said one or more nucleosides other than tc-DNA nucleosides are independently of each other 2′-O-methoxyethyl-RNA nucleosides.

In some embodiments, said oligomeric compound further comprises one or more nucleosides other than tc-DNA nucleosides, wherein said one or more nucleosides other than tc-DNA nucleosides are independently of each other morpholino nucleosides.

In some embodiments, said oligomeric compound further comprises one or more nucleosides other than tc-DNA nucleosides, wherein said one or more nucleosides other than tc-DNA nucleosides are independently of each other locked nucleic acid RNA nucleosides.

### Internucleosidic Linkage Groups

In an embodiment, the internucleosidic linkage group of the oligomeric compounds is independently selected from the group consisting of a phosphorothioate linkage, a phosphorodithioate linkage, a phosphorodiester linkage, a phosphotriester linkage, an aminoalkylphosphotriester linkage, a methyl phosphonate linkage, an alkyl phosphonate linkage, a 5′-alkylene phosphonate linkage, a phosphonate linkage, a phosphinate linkage, a phosphoramidate linkage, an 3′-aminophosphoramidate linkage, an aminoalkyl phosphoramidate linkage, a thionophosphoramidate linkage, a thionoalkylphosphonate linkage, a thionoalkylphosphotriester linkage, a selenophosphate linkage, and a boranophosphate linkage.

In some embodiments, the internucleosidic linkages of the oligomeric compounds are independently selected from the group consisting of a phosphorothioate linkage and a phosphorodiester linkage. In an embodiment, the internucleosidic linkages of the oligomeric compounds comprise only phosphorodiester linkages.

An exemplary phosphorothioate linkage is shown in Formula (16):

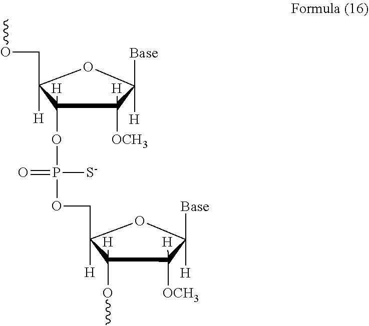

An exemplary phosphorodiester linkage is shown in Formula (17):

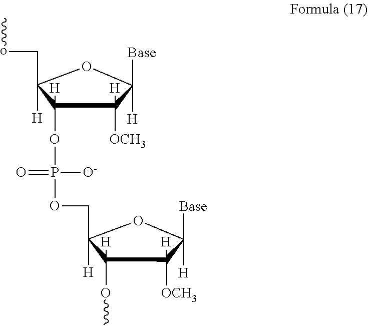

The wavy lines in Formula (16) and Formula (17) represent additional oligomeric repeating nucleoside and internucleosidic linkages as described herein.

General methods of preparation of internucleosidic linkages for use with oligomeric compounds are known in the art, including the methods described in U.S. Pat. Nos. 3,687,808; 4,469,863; 4,476,301; 5,023,243; 5,177,196; 5,188,897; 5,264,423; 5,276,019; 5,278,302; 5,286, 717; 5,321, 131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5, 194,599; 5,565,555; 5,527,899; 5,721,218; 5,672,697 and 5,625,050, the disclosures of which are incorporated by reference herein. Phosphorothioates may be prepared from phosphate triesters, for example, using phenylacetyl disulfide (PADS) chemistry described in Krotz et al, Org. Proc. R&D 2004, 8, 852-58, as part of solid-phase syntheses using, e.g., the four-reaction 3′- to 5′-elongation cycle (detritylation, coupling, sulfurization using PADS, and capping, followed by deprotection, cleavage from the support, and purification steps.

The term “phosphorus moiety”, as used herein, refers to a moiety comprising a phosphorus atom in the P^{III} or P^{V} valence state and which is represented by Formula (18):

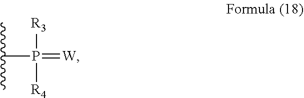

wherein
W represents O, S or Se or W represents an electron pair;
R₃ and R₄ are independently of each other H, halogen, OH, OR₅, NR₆R₇, SH, SR₈, C₁-C₆alkyl, C₁-C₆haloalkyl, C₁-C₆alkoxy, C₁-C₆haloalkoxy, C₁-C₆aminoalkyl; wherein R₅ is C₁-C₉alkyl, C₁-C₆alkoxy, each independently of each other optionally substituted with cyano, nitro, halogen, —NHC(O)C₁-C₃alkyl, —NHC(O)C₁-C₃haloalkyl, C₁-C₃alkylsulfonyl; aryl, C₁-C₆alkylenearyl, C₁-C₆alkylenediaryl, each independently of each other optionally substituted with cyano, nitro, halogen, C₁-C₄alkoxy, C₁-C₄haloalkyl, C₁-C₄haloalkoxy, NHC(O)C₁-C₃alkyl, NHC(O)C₁-C₃haloalkyl, C₁-C₃alkylsulfonyl; acetyl; a hydroxyl protecting group; wherein R₆ and R₇ are independently of each other hydrogen, C₁-C₉alkyl optionally substituted with cyano, nitro, halogen, C₂-C₆alkenyl, C₃-C₆cycloalkyl, C₁-C₃alkoxy; aryl optionally substituted with cyano, nitro, halogen, C₁-C₃alkyl, C₁-C₃alkoxy; an amino protecting group; or together with the nitrogen atom to which they are attached form a heterocyclic ring, wherein preferably said heterocyclic ring is selected from pyrollidinyl, piperidinyl, morpholinyl, piperazinyl and homopiperazine, wherein said heterocyclic ring is optionally substituted with C₁-C₃ alkyl; and wherein R₅ is a thiol protecting group; and wherein the wavy line indicates the attachment to the oxygen of said OR₂ group in any one of Formulas (1) to (14) or in analogous manner for nucleosides not explicitly shown herein by formula. When W represents O, S or Se then said P atom within said phosphorus moiety is in its P^{V} valence state. When W represents an electron pair then said P atom within said phosphorus moiety is in its P^{III} valence. The moiety of Formula (18) includes any possible stereoisomer. Further included in said moieties represented by Formula (18) are salts thereof, wherein typically and preferably said salts are formed upon treatment with inorganic bases or amines, and are typically and preferably salts derived from reaction with the OH or SH groups being (independently of each other) said R₃ and R₄. Preferred inorganic bases or amines leading to said salt formation with the OH or SH groups are well known in the art and are typically and preferably trimethylamine, diethylamine, methylamine or ammonium hydroxide. These phosphorus moieties included in the present invention are, if appropriate, also abbreviated as “O⁻HB⁺”, wherein said HB⁺ refers to the counter cation formed.

The term “phosphorus moiety”, as used herein, includes and, typically and preferably is independently at each occurrence selected from a moiety derived from phosphonates, phosphite triester, monophosphate, diphosphate, triphosphate, phosphate triester, phosphate diester, thiophosphate ester, di-thiophosphate ester or phosphoramidites. Thus, in an embodiment, said OR₂ in any one of the Formula (1) to (14) or in analogous manner for nucleosides not explicitly shown herein by formula, is independently at each occurrence selected from phosphonates, phosphite triester, monophosphate, diphosphate, triphosphate, phosphate triester, phosphate diester, thiophosphate ester, di-thiophosphate ester or phosphoramidites. Further phosphorus moieties usable in the present invention are disclosed in Tetrahedron Report Number 309 (Beaucage and Lyer, Tetrahedron, 1992, 48, 2223-2311), the disclosure of which is incorporated herein by reference.

The term “phosphorus moiety”, as used herein, preferably refers to a group R₂ as defined in any one of the Formulae (1) to (14) or in analogous manner for nucleosides not explicitly shown herein by any formula, comprising a phosphorus atom in the P^{III} or P^{V} valence state and which is represented independently at each occurrence either by Formula (19), Formula (20) or Formula (21),

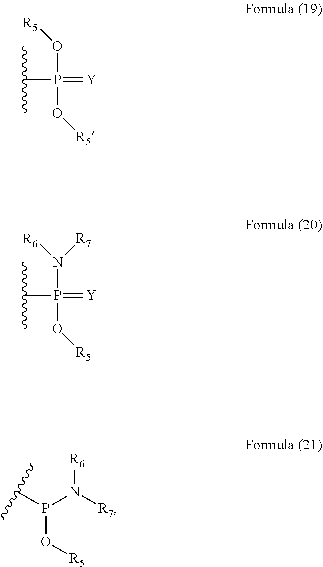

wherein Y is O, S or Se, and wherein Y preferably is O or S, more preferably Y is 0; and wherein Rₛ and R₅, are independently at each occurrence and of each other hydrogen, C₁-C₉alkyl, C₁-C₆alkoxy, each independently of each other optionally substituted with cyano, nitro, halogen, —NHC(O)C₁-C₃alkyl, —NHC(O)C₁-C₃haloalkyl, C₁-C₃alkylsulfonyl; aryl, C₁-C₆alkylenearyl, C₁-C₆alkylenediaryl each independently of each other optionally substituted with cyano, nitro, halogen, C₁-C₄alkoxy, C₁-C₄haloalkyl, C₁-C₄haloalkoxy, —NHC(O)C₁-C₃alkyl, NHC(O)C₁-C₃haloalkyl, C₁-C₃alkylsulfonyl; a hydroxyl protecting group; wherein R₆ and R₇ are independently of each other hydrogen, C₁-C₉alkyl optionally substituted with cyano, nitro, halogen, C₂-C₆alkenyl, C₃-C₆cycloalkyl, C₁-C₃alkoxy; aryl, preferably phenyl, optionally substituted with cyano, nitro, halogen, C₁-C₃ alkyl, C₁-C₃alkoxy; an amino protecting group; or together with the nitrogen atom to which they are attached form a heterocyclic ring, wherein preferably said heterocyclic ring is selected from pyrollidinyl, piperidinyl, morpholinyl, piperazinyl and homopiperazine, wherein said heterocyclic ring is optionally substituted with C₁-C₃ alkyl; and wherein R₅ is a thiol protecting group; and wherein the wavy line indicates the attachment to the oxygen of said OR₂ group in any one of the Formulae (1) to (5).

Advantageously, in a particular embodiment, the oligomeric compound can comprise one or more and preferably several tricyclo-deoxyribonucleic acid (tc-DNA) nucleosides and at least one modified ribonucleic acid nucleoside and advantageously only one modified ribonucleic acid nucleoside. The latter can be present anywhere in the sequence of the oligomeric compound according to the present invention.

In particular, the oligomeric compound according to the present invention comprises or consists of one of the following nucleotide sequences:
5′-GGAGATgGCAGTTTC-3′ (SEQ ID NO: 4 in the appended sequence listing),
5′-GGAGATGgCAGTTTC-3′ (SEQ ID NO: 5 in the appended sequence listing),
5′-GGAGATGGcAGTTTC-3′ (SEQ ID NO: 6 in the appended sequence listing), and
5′-GGAGATGGCaGTTTC-3′ (SEQ ID NO: 7 in the appended sequence listing). in which tcDNA nucleotides are typed in capital letters while the modified ribonucleic acid nucleoside as previously defined is typed in lowercase letter. This typing applies to all sequences thereinafter.

In particular, the modified RNA nucleoside at positions +7, +8, +9 and +10, respectively in the above sequences is a 2′-modified RNA nucleoside and, more particularly, a 2′-O-methyl RNA nucleoside.

In this last alternative, when the oligomeric compound consists of a nucleotide sequence corresponding to the sequence SEQ ID NO: 4, SEQ ID NO: 5, SEQ ID NO: 6 or SEQ ID NO: 7, this compound is designed as REGONE.7, REGONE.8, REGONE.9 or REGONE.10 respectively.

Advantageously, in some embodiments, the 15 monomer subunits in the nucleotide sequence corresponding to the sequence SEQ ID NO: 4, SEQ ID NO: 5, SEQ ID NO: 6 or SEQ ID NO: 7 in the appended sequence listing are linked by phosphodiester (PO) bonds.

In some embodiments, the oligomeric compound comprises at least one nucleotide sequence having at least 70%, at least 73%, at least 75%, at least 80%, at least 85%, at least 90%, at least 93%, at least 95%, or at least 99% identity with SEQ ID NO: 4. In some embodiments, the oligomeric compound comprises at least one nucleotide sequence identical to SEQ ID NO: 4.

In some embodiments, the oligomeric compound comprises at least one nucleotide sequence having at least 70%, at least 73%, at least 75%, at least 80%, at least 85%, at least 90%, at least 93%, at least 95%, or at least 99% identity with SEQ ID NO: 5. In some embodiments, the oligomeric compound comprises at least one nucleotide sequence identical to SEQ ID NO: 5.

In some embodiments, the oligomeric compound comprises at least one nucleotide sequence having at least 70%, at least 73%, at least 75%, at least 80%, at least 85%, at least 90%, at least 93%, at least 95%, or at least 99% identity with SEQ ID NO: 6. In some embodiments, the oligomeric compound comprises at least one nucleotide sequence identical to SEQ ID NO: 6.

In some embodiments, the oligomeric compound comprises at least one nucleotide sequence having at least 70%, at least 73%, at least 75%, at least 80%, at least 85%, at least 90%, at least 93%, at least 95%, or at least 99% identity with SEQ ID NO: 7. In some embodiments, the oligomeric compound comprises at least one nucleotide sequence identical to SEQ ID NO: 7.

In a particular embodiment, the oligomeric compound according to the present invention can be combined with one or more lipid moieties. In other words, one or more lipid moieties can be covalently linked to the oligomeric compound either directly or indirectly i.e. via a spacer.

In some embodiments, the oligomeric compound according to the present invention can be combined with one or more lipid moiety, preferably exactly one lipid moiety, wherein said one or more lipid moiety is covalently linked to said oligomeric compound either directly or via a spacer.

Any lipid moiety can be implemented in the present invention.

In one embodiment, said one or more lipid moiety is independently of each other selected from a fatty acid moiety, a fatty diacid moiety, a glycerolipid moiety, a glycerophospholipid moiety, a sphingolipid moiety, a phospholipid, an alkylphosphate moiety and an alkylphosphonate moiety.

In one embodiment, said one or more lipid moiety is independently of each other selected from a fatty acid moiety, a fatty diacid moiety, a phospholipid, an alkylphosphate moiety and an alkylphosphonate moiety.

In one preferred embodiment, said one or more lipid moiety is independently of each other a fatty acid moiety. In some embodiments, said one or more lipid moiety is independently of each other a fatty diacid moiety. In another embodiment, said one or more lipid moiety is independently of each other a glycerolipid moiety. In another embodiment, said one or more lipid moiety is independently of each other a glycerophospholipid moiety. In another embodiment, said one or more lipid moiety is independently of each other a sphingolipid moiety. In some embodiments, said one or more lipid moiety is independently of each other an alkylphosphate moiety. In some embodiments, said one or more lipid moiety is independently of each other an alkylphosphonate moiety.

In one embodiment, the one or more lipid moiety is negatively charged at pH of 7.4, wherein typically said pH of 7.4 corresponds to the physiological pH.

In some embodiments, said one or more lipid moiety is independently of each other selected from a fatty acid moiety, a fatty diacid moiety, an alkylphosphate moiety and an alkylphosphonate moiety.

In some embodiments, said one or more lipid moiety is independently of each other a fatty acid moiety or a fatty diacid moiety.

In some embodiments, said one or more lipid moiety is independently of each other a fatty acid moiety, wherein said fatty acid moiety is a saturated fatty acid moiety. In some embodiments, said one or more lipid moiety is independently of each other a fatty acid moiety, wherein said fatty acid moiety is an unsaturated fatty acid moiety.

In some embodiments, said one or more lipid moiety is independently of each other a fatty diacid moiety, wherein said fatty diacid moiety is a saturated fatty diacid moiety. In some embodiments, said one or more lipid moiety is independently of each other a fatty diacid moiety, wherein said fatty acid moiety is an unsaturated fatty diacid moiety.

In a very preferred embodiment, said one or more lipid moiety is independently of each other a fatty acid moiety, wherein said fatty acid moiety is a saturated unbranched fatty acid moiety.

In some embodiments, said one or more lipid moiety is independently of each other a fatty acid moiety, wherein said fatty acid moiety is derived from a saturated unbranched fatty acid. In some embodiments, said one or more lipid moiety is independently of each other a fatty diacid moiety, wherein said fatty diacid moiety is derived from a saturated unbranched fatty diacid.

In a very preferred embodiment, said one or more lipid moiety is independently of each other a fatty acid moiety or a fatty diacid moiety, wherein said fatty acid moiety is a saturated unbranched fatty acid moiety, and wherein said fatty diacid moiety is a saturated unbranched fatty diacid moiety.

In some embodiments, said one or more lipid moiety is independently of each other a moiety of formula (I):

A—B—* (I)
wherein A is C₃₋₃₂alkyl, C₃₋₃₂alkenyl, C₃₋₃₂alkynyl, HOOC—C₃₋₃₂alkylene, HOOC—C₃₋₃₂alkenylene or HOOC—C₃₋₃₂alkynylene, and B is C(O), OP(OH), OP(O)(OH), OP(O)(SH), NH—C(O), NH—P(O)(OH), NH—P(O)(SH) or a pharmaceutically acceptable salt thereof; and wherein said asterisk (*) represents the point of said covalent linkage to said oligomeric compound or to said spacer.

In a further preferred embodiment, said one or more lipid moiety is independently of each other selected from any one of the formulae (a) to (u): a. C₃₋₃₂alkyl-C(O)—*,
b. C₃₋₃₂alkenyl-C(O)—*,
c. C₃₋₃₂alkynyl-C(O)—*,
d. C₃₋₃₂alkyl-OP(OH)—*,
e. C₃₋₃₂alkenyl-OP(OH)—*,
f. C₃₋₃₂alkynyl-OP(OH)—*,
g. C₃₋₃₂alkyl-OP(O)(OH)—*,
h. C₃₋₃₂alkenyl-OP(O)(OH)—*,
i. C₃₋₃₂alkynyl-OP(O)(OH)—*,
j. C₃₋₃₂alkyl-OP(O)(SH)—*,
k. C₃₋₃₂alkenyl-OP(O)(SH)—*,
I. C₃₋₃₂alkynyl-OP(O)(SH)—*,
m. C₃₋₃₂alkyl-NH—C(O)—*,
n. C₃₋₃₂alkenyl-NH—C(O)—*,
o. C₃₋₃₂alkynyl-NH—C(O)—*,
p. C₃₋₃₂alkyl-NH—P(O)(OH)—*,
q. C₃₋₃₂alkenyl-NH—P(O)(OH)—*,
r. C₃₋₃₂alkynyl-NH—P(O)(OH)—*,
s. HOOC—C₃₋₃₂alkylene-C(O)—*,
t. HOOC—C₃₋₃₂alkenylene-C(O)—*, and
u. HOOC—C₃₋₃₂alkynylene-C(O)—*,
wherein said asterisk (*) represents the point of said covalent linkage to said oligomeric compound or to said spacer.

In some embodiments, said one or more lipid moiety is independently of each other a moiety of formula C₃₋₃₂alkyl-C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkenyl-C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkynyl-C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkyl-OP(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkenyl-OP(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkynyl-OP(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkyl-OP(O)(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkenyl-OP(O)(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkynyl-OP(O)(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkyl-OP(O)(SH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkenyl-OP(O)(SH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkynyl-OP(O)(SH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkyl-NH—C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkenyl-NH—C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkynyl-NH—C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkyl-NH—P(O)(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkenyl-NH—P(O)(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula C₃₋₃₂alkynyl-NH—P(O)(OH)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula HOOC—C₃₋₃₂alkylene-C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula HOOC—C₃₋₃₂alkenylene-C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety of formula HOOC—C₃₋₃₂alkynylene-C(O)—*, wherein said asterisk (*) represents the point of said covalent linkage to said oligomeric compound or to said spacer, wherein preferably said composition comprises exactly one lipid moiety.

In some embodiments, said one or more lipid moiety is independently of each other a moiety from any one of the formulae (a) to (d):
a. C₃₋₃₂alkyl-C(O)—*,
b. HOOC—C₃₋₃₂alkylene-C(O)—*,
c. C₃₋₃₂alkyl-OP(O)(OH)—*
d. C₃₋₃₂alkyl-OP(O)(SH)—*
wherein said asterisk (*) represents the point of said covalent linkage to said oligomeric compound or to said spacer, and wherein preferably said C₃₋₃₂alkyl is an unbranched C₃₋₃₂alkyl, and wherein further preferably said C₃₋₃₂alkyl is an unbranched C₃₋₃₂alkyl having an uneven number of carbon atoms, and wherein preferably said C₃₋₃₂alkylene is an unbranched C₃₋₃₂alkylene, and wherein further preferably said C₃₋₃₂alkylene is an unbranched C₃₋₃₂alkylene having an uneven number of carbon atoms.

In a further preferred embodiment, said one or more lipid moiety is independently of each other a moiety of formula C₃₋₃₂alkyl-C(O)—*, wherein said asterisk (*) represents the point of said covalent linkage to said oligomeric compound or to said spacer, and wherein preferably said C₃₋₃₂alkyl is an unbranched C₃₋₃₂alkyl, and wherein further preferably said C₃₋₃₂alkyl is an unbranched C₃₋₃₂alkyl having an uneven number of carbon atoms.

In some embodiments, said one or more lipid moiety is independently of each other a moiety of formula C₃₋₃₂alkyl-C(O)—*, wherein said asterisk (*) represents the point of said covalent linkage to said oligomeric compound or to said spacer, wherein preferably said composition comprises exactly one lipid moiety, and wherein said C₃₋₃₂alkyl is an unbranched C₃₋₃₂alkyl.

In some embodiments, said one or more lipid moiety is independently of each other a moiety of formula C₃₋₃₂alkyl-C(O)—*, wherein said asterisk (*) represents the point of said covalent linkage to said oligomeric compound or to said spacer, wherein preferably said composition comprises exactly one lipid moiety, and wherein said C₃₋₃₂alkyl is an unbranched C₃₋₃₂alkyl having an uneven number of carbon atoms.

In some embodiments, said one or more lipid moiety is independently of each other a moiety of formula C₃₋₃₂alkenyl-C(O)—*, wherein said asterisk (*) represents the point of said covalent linkage to said oligomeric compound or to said spacer, and wherein preferably said C₃₋₃₂alkenyl is a branched C₃₋₃₂alkenyl, and wherein further preferably said C₃₋₃₂alkenyl is a branched C₃₋₃₂alkenyl having an uneven number of carbon atoms.

In some embodiments, said one or more lipid moiety is independently of each other a moiety of formula C₃₋₃₂alkenyl-C(O)—*, wherein said asterisk (*) represents the point of said covalent linkage to said oligomeric compound or to said spacer, wherein preferably said composition comprises exactly one lipid moiety, and wherein said C₃₋₃₂alkenyl is a branched C₃₋₃₂alkenyl.

In some embodiments, said one or more lipid moiety is independently of each other a moiety of formula C₃₋₃₂alkenyl-C(O)—*, wherein said asterisk (*) represents the point of said covalent linkage to said oligomeric compound or to said spacer, wherein preferably said composition comprises exactly one lipid moiety, and wherein said C₃-32alkenyl is a branched C₃₋₃₂alkenyl having an uneven number of carbon atoms.

In some embodiments, said one or more lipid moiety is independently of each other a saturated C₈₋₂₆-fatty acid moiety, wherein preferably said saturated C₈₋₂₆-saturated fatty acid moiety is derived from caprylic acid (C8), capric acid (C10), lauric acid (C12), myristic acid (C14), palmitic acid (C16), stearic acid (C18), arachidic acid (C20), lignoceric acid (C22) or cerotic acid (C24).

In some embodiments, said one or more lipid moiety is independently of each other a saturated fatty acid moiety, wherein said saturated fatty acid moiety is derived from caprylic acid (C8), capric acid (C10), lauric acid (C12), myristic acid (C14), palmitic acid (C16), stearic acid (C18), arachidic acid (C20), lignoceric acid (C22) and cerotic acid (C24).

In some embodiments, said one or more lipid moiety is independently of each other a saturated fatty acid moiety derived from palmitic acid (C16) or stearic acid (C18), wherein preferably said one or more lipid moiety is a saturated fatty acid moiety derived from palmitic acid (C16).

In some embodiments, said one or more lipid moiety is independently of each other an unsaturated C14-22-fatty acid moiety, wherein preferably said unsaturated C14-22-fatty acid moiety is derived from myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid. In some embodiments, said one or more lipid moiety is a saturated fatty acid moiety derived from palmitoleic acid.

In some embodiments, said one or more lipid moiety is independently of each other an unsaturated fatty acid moiety, wherein said unsaturated fatty acid moiety is derived from myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

In some embodiments, said one or more lipid moiety is an unsaturated fatty acid moiety derived from oleic acid.

In some embodiments, said one or more lipid moiety is independently of each other a moiety of formula (HOOC)—C₃₋₃₂alkylene-C(O)—*, wherein said asterisk (*) represents the point of said covalent linkage to said oligomeric compound or to said spacer, and wherein preferably said C₃₋₃₂alkylene is an unbranched C₃₋₃₂alkylene, and wherein further preferably said C₃₋₃₂alkylene is an unbranched C₃₋₃₂alkylene having an uneven number of carbon atoms.

In some embodiments, said one or more lipid moiety is independently of each other a moiety of formula (HOOC)—(CH₂)ᵣ—(CH)(C₅₋₂₅alkyl)—(CH₂)ₜ—C(O)—*, wherein said asterisk (*) represents the point of said covalent linkage to said oligomeric compound or to said spacer, wherein r is independently of each other an integer of 1 to 3, wherein t is independently of each other an integer of 1 to 3.

In some embodiments, said one or more lipid moiety is independently of each other a moiety of formula, (HOOC)—(CH₂)ᵣ—(CH)[(CH₂)₅CH₃]—(CH₂)ₜ—C(O)—*, wherein said asterisk (*) represents the point of said covalent linkage to said oligomeric compound or to said spacer, and wherein r is independently of each other an integer of 1 to 3, wherein s is independently of each other an integer of 4 to 24, wherein t is independently of each other an integer of 1 to 3.

In some embodiments, said one or more lipid moiety is independently of each other a moiety of formula, (HOOC)—(CH2)ᵣ—(CH)[(CH₂)₅CH₃]—(CH₂)ₜ—C(O)—*, wherein said asterisk (*) represents the point of said covalent linkage to said oligomeric compound or to said spacer, and wherein r is independently of each other an integer of 1 or 2, wherein s is independently of each other an integer of 5 to 19, wherein t is independently of each other an integer of 1 or 2.

In some embodiments, said one or more lipid moiety is independently of each other a moiety of formula, (HOOC)—(CH₂)ᵣ—(CH)[(CH₂)₅CH₃]—(CH₂)ₜ—C(O)—*, wherein said asterisk (*) represents the point of said covalent linkage to said oligomeric compound or to said spacer, and wherein r is 1, wherein s is independently of each other an integer of 4 to 24, preferably 5 to 19, wherein t is 1.

In some embodiments, said one or more lipid moiety is independently of each other a moiety of formula, (HOOC)—(CH₂)ᵣ—(CH)[(CH₂)₅CH₃]—(CH₂)ₜ—C(O)—*, wherein said asterisk (*) represents the point of said covalent linkage to said oligomeric compound or to said spacer, and wherein r is 1, wherein s is independently of each other an integer of 5 to 19, preferably 11 to 17, wherein t is 1.

In some embodiments, said one or more lipid moiety is independently of each other a moiety of formula, (HOOC)—(CH₂)ᵣ—(CH)[(CH₂)₅CH₃]—(CH₂)ₜ—C(O)—*, wherein said asterisk (*) represents the point of said covalent linkage to said oligomeric compound or to said spacer, and wherein r is 1, wherein s is 15, wherein t is 1.

Thus, in some embodiments, said one or more lipid moiety is 3-pentadecylglutaric acid (PDG).

In some embodiments, said lipid moiety is linked directly to said oligomeric compound.

In some embodiments, said one or more lipid moiety is linked to said oligomeric compound via a spacer.

In an embodiment, said spacer has from 5 to 30 C-atoms, preferably from 5 to 25 C-atoms, more preferably from 5 to 20 C-atoms, or most preferably from 5 to 17 C-atoms. In additional embodiments, said spacer has from 4 to 20 hetero-atoms, preferably from 4 to 18 hetero-atoms, more preferably from 4 to 14 hetero-atoms, or most preferably from 4 to 12 hetero-atoms. Particularly preferred examples of hetero-atoms are N—, and O-atoms. H-atoms are not hetero-atoms.

In some embodiments, said spacer comprises, preferably is, independently selected from, any one of the formulae:
#—NH—C₂₋₁₂al kylene-§ ,
#—NH—C₂₋₁₂al kylene-OP(OH)-§ ,
#—NH—C₂₋₁₂al kylene-OP(O)(SH)-§ ,
#—NH—C₂₋₁₂al kylene-OP(O)(OH)-§ ,
#—SH—C₂₋₁₂al kylene-§ ,
#—NH—C₂₋₁₂alkylene-NH—C(O)-§ ,
#—NH—C₂₋₁₂alkylene-NH—P(O)(OH)-§ , and
#—NH—C₂₋₁₂alkylene-NH—P(O)(SH)-§ ,
wherein one or more —CH₂-moieties in said C₂₋₁₂alkylene are optionally replaced independently by —O—, —S—, —NH—, —C(O)—, —C(O)O—, an aryl, a heteroaryl, a cycloalkyl, a heterocycloalkyl, —OP(OH)O—, OP(O)(SH)O—, OP(O)(OH)O—, NHP(O)(OH)O—, NHP(O)(SH)O—, or —(O—CH₂—CH₂)ₖ— with k being an integer of 1 to 8, and wherein one or more —CH₂-moieties in said C₂₋₁₂alkylene are independently of each other optionally substituted with one or more —COOH, —NH₂, —OP(O)(OH)₂ or —OH (and thus meaning that one or both, preferably one, of the hydrogen atoms in one or more of the —CH₂—moieties in said C₂₋₁₂alkylene are independently of each other optionally substituted with one or more —COOH, —NH₂, —OP(O)(OH)₂ or —OH), and wherein said (#) represents the point of covalent linkage to said lipid moiety and said (§ ) represents the point of covalent linkage to said oligomeric compound.

In a very preferred embodiment, said spacer comprises, preferably is, independently selected from, any one of the formulae:
a. #—NH—C₂₋₁₂alkylene-§ ,
b. #—NH—C₂₋₁₂alkylene-OP(OH)-§ ,
c. #—NH—C₂₋₁₂alkylene-OP(O)(SH)-§ ,
d. #—NH—C₂₋₁₂alkylene-OP(O)(OH)-§ ,
e. #—NH—C₂₋₁₂alkylene-NH—C(O)-§ ,
f. #—NH—C₂₋₁₂alkylene-NH—P(O)(OH)-§ , and
g. #—NH—C₂₋₁₂alkylene-NH—P(O)(SH)-§ ,
wherein said (#) represents the point of covalent linkage to said lipid moiety and said (§ ) represents the point of covalent linkage to said oligomeric compound.

In a very preferred embodiment, said spacer comprises, preferably is, independently selected from, any one of the formulae:
a. #—NH—C₂₋₁₂alkylene-§ ,
b. #—NH—C₂₋₁₂alkylene-OP(OH)-§ ,
c. #—NH—C₂₋₁₂alkylene-OP(O)(SH)-§ ,
d. #—NH—C₂₋₁₂alkylene-OP(O)(OH)-§ ,
e. #—NH—C₂₋₁₂alkylene-NH—C(O)-§ ,
f. #—NH—C₂₋₁₂alkylene-NH—P(O)(OH)-§ , and
g. #—NH—C₂₋₁₂alkylene-NH—P(O)(SH)-§ ,
wherein one or more —CH₂—moieties in said C₂₋₁₂alkylene are optionally replaced independently by —O—, —S—, —NH—, —C(O)—, —C(O)O—, an aryl, a heteroaryl, a cycloalkyl, a heterocycloalkyl, —OP(OH)O—, OP(O)(SH)O—, OP(O)(OH)O—, NHP(O)(OH)O—, NHP(O)(SH)O—, or —(O—CH₂—CH₂)ₖ—with k being an integer of 1 to 8, and wherein said (#) represents the point of covalent linkage to said lipid moiety and said (§ ) represents the point of covalent linkage to said oligomeric compound.

In a very preferred embodiment, said spacer comprises, preferably is, independently selected from, any one of the formulae:
a. #—NH—C₂₋₁₂alkylene-§ ,
b. #—NH—C₂₋₁₂alkylene-OP(OH)-§ ,
c. #—NH—C₂₋₁₂alkylene-OP(O)(SH)-§ ,
d. #—NH—C₂₋₁₂alkylene-OP(O)(OH)-§ ,
e. #—NH—C₂₋₁₂alkylene-NH—C(O)-§ ,
f. #—NH—C₂₋₁₂alkylene-NH—P(O)(OH)-§ , and
g. #—NH—C₂₋₁₂alkylene-NH—P(O)(SH)-§ ,
wherein one or more —CH₂—moieties in said C₂₋₁₂alkylene are optionally replaced independently by —O—, —S—, —NH—, —C(O)—, —C(O)O—, an aryl, a heteroaryl, a cycloalkyl, a heterocycloalkyl, —OP(OH)O—, OP(O)(SH)O—, OP(O)(OH)O—, NHP(O)(OH)O—, NHP(O)(SH)O—, or —(O—CH₂—CH₂)ₖ— with k being an integer of 1 to 8, and wherein one or more —CH₂—moieties in said C₂₋₁₂alkylene are independently of each other optionally substituted with one or more —COOH, —NH₂, —OP(O)(OH)₂ or —OH, and wherein said (#) represents the point of covalent linkage to said lipid moiety and said (§ ) represents the point of covalent linkage to said oligomeric compound.

In a very preferred embodiment, said spacer comprises, preferably is, independently selected from, any one of the formulae:
a. #—NH—C₂₋₁₂alkylene-§ ,
b. #—NH—C₂₋₁₂alkylene-OP(OH)-§ ,
c. #—NH—C₂₋₁₂alkylene-OP(O)(SH)-§ ,
d. #—NH—C₂₋₁₂alkylene-OP(O)(OH)-§ ,
e. #—NH—C₂₋₁₂alkylene-NH—C(O)-§ ,
f. #—NH—C₂₋₁₂alkylene-NH—P(O)(OH)-§ , and
g. #—NH—C₂₋₁₂alkylene-NH—P(O)(SH)-§ ,
wherein one or more —CH₂—moieties in said C₂₋₁₂alkylene are optionally replaced independently by —O—, —S—, —NH—, —C(O)—, —C(O)O—, phenyl, triazolyl, cyclopentyl, cyclohexyl, succinimidyl, —OP(OH)O—, OP(O)(SH)O—, OP(O)(OH)O—, NHP(O)(OH)O—, NHP(O)(SH)O—, or —(O—CH₂—CH₂)ₖ— with k being an integer of 1 to 8, and wherein one or more —CH₂—moieties in said C₂₋₁₂alkylene are independently of each other optionally substituted with one or more —COOH, —NH₂, —OP(O)(OH)₂ or —OH, and wherein said (#) represents the point of covalent linkage to said lipid moiety and said (§ ) represents the point of covalent linkage to said oligomeric compound.

In some embodiments, said spacer comprises, preferably is, independently selected from any one of the formulae:
a. —NH—(CH₂)ₘ—,
b. —NH—(CH₂)ₘ—X—,
c. —NH—(CH₂)ₙ—(O—CH₂—CH₂)ₖ—O—(CH₂)ₚ—,
d. —NH—(CH₂)ₙ—(O—CH₂—CH₂)ₖ—O—(CH₂)ₚ—X—,
e. —NH—CH(COOH)—(CH₂)_{q}—,
f. —NH—CH(COOH)—(CH₂)_{q}—X—,
g. —NH—CH(COOH)—(CH₂)_{q}—C(O)—NH—(CH₂)ₘ—,
h. —NH—CH(COOH)—(CH₂)_{q}—C(O)—NH—(CH₂)ₘ—X—,
i. —NH—CH(COOH)—(CH₂)_{q}—C(O)—NH—(CH₂)ₙ—(O—CH₂—CH₂)ₖ—O—(CH₂)ₚ—X—,
j. —NH—CH(COOH)—(CH₂)_{q}—C(O)—NH—(CH₂)ₙ—(O—CH₂—CH₂)ₖ—O—(CH₂)ₚ—C(O)—NH—(CH₂)ₙ—(O—CH₂—CH₂)ᵣX—,
wherein X is independently of each other OP(OH), OP(O)(SH) or OP(O)(OH), wherein k is independently of each other an integer of 1 to 8, wherein m is independently of each other an integer of 2 to 12, wherein n is independently of each other an integer of 2 to 4, wherein p is independently of each other an integer of 1 to 5, wherein q is independently of each other an integer of 1 to 3, preferably 1 or 2, wherein r is independently of each other an integer of 1 to 3, preferably 1 or 2.

In some embodiments, said spacer comprises, preferably is, independently selected from any one of the formulae:
a. #—NH—(CH₂)ₘ—§ ,
b. #—NH—(CH₂)ₘ—X-§ ,
c. #—NH—(CH₂)ₙ—(O—CH₂—CH₂)ₖ—O—(CH₂)ₚ—§ ,
d. #—NH—(CH₂)ₙ—(O—CH₂—CH₂)ₖ—O—(CH₂)ₚ—X-§ ,
e. #—NH—CH(COOH)—(CH₂)_{q}—§ ,
f. #—NH—CH(COOH)—(CH₂)_{q}—X-§ ,
g. #—NH—CH(COOH)—(CH₂)_{q}—C(O)—NH—(CH₂)ₘ—§ ,
h. #—NH—CH(COOH)—(CH₂)_{q}—C(O)—NH—(CH₂)ₘ—X-§ ,
i. #—NH—CH(COOH)—(CH₂)_{q}—C(O)—NH—(CH₂)ₙ—(O—CH₂—CH₂)ₖ—O—(CH₂)ₚ—X-§ ,
j. #—NH—CH(COOH)—(CH₂)_{q}—C(O)—NH—(CH₂)ₙ—(O—CH₂—CH₂)ₖ—O—(CH₂)ₚ—C(O)—NH—(CH₂)ₙ—(O—CH₂—CH₂)ᵣ—X-§ ,
wherein X is independently of each other OP(OH), OP(OH)(S) or OP(O)(OH), wherein k is independently of each other an integer of 1 to 8, wherein m is independently of each other an integer of 2 to 12, wherein n is independently of each other an integer of 2 to 4, wherein p is independently of each other an integer of 1 to 5, wherein q is independently of each other an integer of 1 to 3, preferably 1 or 2, wherein r is independently of each other an integer of 1 to 3, preferably 1 or 2, and wherein said (#) represents the point of covalent linkage to said lipid moiety and said (§ ) represents the point of covalent linkage to said oligomeric compound.

In some embodiments, said spacer comprises, preferably is, independently selected from any one of the formulae:
a. —Z—NH—(CH₂)ₘ—X—
b. —Z—NH—(CH₂)ₙ—(O—CH₂—CH₂)ₖ—O—(CH₂)ₚ—X—
c. —Z[—NH—(CH₂)ₙ—(O—CH₂—CH₂)ₖ—O—(CH₂)ₚ—C(O)—]—NH—(CH₂)_{q}—X—
d. —Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X—
wherein —Z— represents independently of each other a bond or —NH—CH(COOH)—(CH₂)₂—C(O)— or —NH—CH[(CH₂)₂COOH]—C(O)—, wherein X is independently of each other OP(OH), OP(O)(SH), OP(O)(OH), NHP(O)(OH), NHP(O)(SH) or NH—C(O), wherein k is independently of each other an integer of 1 to 8, wherein m is independently of each other an integer of 2 to 12, wherein n is independently of each other an integer of 2 to 4, and wherein p is independently of each other an integer of 1 to 5, wherein q is independently of each other an integer of 1 to 6, preferably 3 or 6, wherein r is independently of each other an integer of 1 to 3, preferably 1 or 2.

In some embodiments, said spacer comprises, preferably is, independently selected from any one of the formulae:
a. #—Z—NH—(CH₂)ₘ—X-§
b. #—Z—NH—(CH₂)ₙ—(O—CH₂—CH₂)ₖ—O—(CH₂)ₚ—X-§
c. #—Z[—NH—(CH₂)ₙ—(O—CH₂—CH₂)ₖ—O—(CH₂)ₚ—C(O)—]—NH—(CH₂)_{q}—X-§
d. #—Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X-§
wherein —Z— represents independently of each other a bond or —NH—CH(COOH)—(CH₂)₂—C(O)— or —NH—CH[(CH₂)₂COOH]—C(O)—, wherein X is independently of each other OP(OH), OP(O)(SH), OP(O)(OH), NHP(O)(OH), NHP(O)(SH) or NH—C(O), wherein k is independently of each other an integer of 1 to 8, wherein m is independently of each other an integer of 2 to 12, wherein n is independently of each other an integer of 2 to 4, and wherein p is independently of each other an integer of 1 to 5, wherein q is independently of each other an integer of 1 to 6, preferably 3 or 6, wherein r is independently of each other an integer of 1 to 3, preferably 1 or 2, and wherein said (#) represents the point of covalent linkage to said lipid moiety and said (§ ) represents the point of covalent linkage to said oligomeric compound.

In some embodiments, the spacer comprises, preferably is, independently selected from any one of the formulae:
a. #—Z—NH—(CH₂)ₘ—X-§
b. #—Z—NH—(CH₂)ₙ—(O—CH₂—CH₂)ₖ—O—(CH₂)ₚ—X-§
c. #—Z[—NH—(CH₂)ₙ—(O—CH₂—CH₂)ₖ—O—(CH₂)ₚ—C(O)—]—NH—(CH₂)_{q}—X-§
d. #—Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X-§
wherein —Z— represents independently of each other a bond or —NH—CH(COOH)—(CH₂)₂—C(O)— or —NH—CH[(CH₂)₂COOH]—C(O)—, wherein X is independently of each other OP(OH), OP(O)(SH), OP(O)(OH), NHP(O)(OH), NHP(O)(SH) or NH—C(O), wherein k is independently of each other an integer of 1 or 2, wherein m is independently of each other an integer of 4 to 8, wherein n is independently of each other an integer of 2 to 4, and wherein p is independently of each other an integer of 1 or 2, wherein q is independently of each other an integer of 1 to 6, wherein r is independently of each other an integer of 1 to 3, preferably 1 or 2, and wherein said (#) represents the point of covalent linkage to said lipid moiety and said (§ ) represents the point of covalent linkage to said oligomeric compound.

In some embodiments, the spacer comprises, preferably is, independently selected from any one of the formulae:
a. #—Z—NH—(CH₂)ₘ—X-§
b. #—Z—NH—(CH₂)ₙ—(O—CH₂—CH₂)ₖ—O—(CH₂)ₚ—X-§
c. #—Z[—NH—(CH₂)ₙ—(O—CH₂—CH₂)ₖ—O—(CH₂)ₚ—C(O)—]—NH—(CH₂)_{q}—X-§
d. #—Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X-§
wherein —Z— represents independently of each other a bond or —NH—CH(COOH)—(CH₂)₂—C(O)— or —NH—CH[(CH₂)₂COOH]—C(O)—, X is independently of each other OP(OH), OP(O)(SH) or OP(O)(OH), wherein k is independently of each other an integer of 1 or 2, wherein m is independently of each other an integer of 4 to 8, wherein n is 2, and wherein p is 1, wherein q is independently of each other an integer of 1 to 6, wherein r is independently of each other an integer of 1 to 3, preferably 1 or 2, and wherein said (#) represents the point of covalent linkage to said lipid moiety and said (§ ) represents the point of covalent linkage to said oligomeric compound.

In some embodiments, the spacer comprises, preferably is, independently selected from any one of the formulae:
a. #—Z—NH—(CH₂)ₘ—X-§
b. #—Z—NH—(CH₂)ₙ—(O—CH₂—CH₂)ₖ—O—(CH₂)ₚ—X-§
c. #—Z[—NH—(CH₂)ₙ—(O—CH₂—CH₂)ₖ—O—(CH₂)ₚ—C(O)—]—NH—(CH₂)_{q}—X-§
d. #—Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X-§
wherein —Z— represents independently of each other a bond or —NH—CH(COOH)—(CH₂)₂—C(O)— or —NH—CH[(CH₂)₂COOH]—C(O)—, X is independently of each other OP(O)(SH) or OP(O)(OH), wherein k is independently of each other an integer of 1 or 2, wherein m is independently of each other an integer of 4 to 8, wherein n is 2, and wherein p is 1, wherein q is independently of each other an integer of 1 to 6, wherein r is independently of each other an integer of 1 to 3, preferably 1 or 2, and wherein said (#) represents the point of covalent linkage to said lipid moiety and said (§ ) represents the point of covalent linkage to said oligomeric compound.

In some embodiments, the spacer comprises, preferably is, independently selected from any one of the formulae:
a. #—Z—NH—(CH₂)ₘ—X-§
b. #—Z—NH—(CH₂)ₙ—(O—CH₂—CH₂)ₖ—O—(CH₂)ₚ—X-§
c. #—Z[—NH—(CH₂)ₙ—(O—CH₂—CH₂)ₖ—O—(CH₂)ₚ—C(O)—]—NH—(CH₂)_{q}—X-§
d. #—Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X-§
wherein —Z— represents independently of each other a bond or —NH—CH(COOH)—(CH₂)₂—C(O)— or —NH—CH[(CH₂)₂COOH]—C(O)—, X is independently of each other OP(O)(SH), wherein k is independently of each other an integer of 1 or 2, wherein m is independently of each other an integer of 4 to 8, wherein n is 2, and wherein p is 1, wherein q is independently of each other an integer of 1 to 6, wherein r is independently of each other an integer of 1 to 3, preferably 1 or 2, and wherein said (#) represents the point of covalent linkage to said lipid moiety and said (§ ) represents the point of covalent linkage to said oligomeric compound.

In some embodiments, the spacer comprises, preferably is, independently selected from any one of the formulae:
a. #—Z—NH—(CH₂)ₘ—X-§
b. #—Z—NH—(CH₂)ₙ—(O—CH₂—CH₂)ₖ—O—(CH₂)ₚ—X-§
c. #—Z[—NH—(CH₂)ₙ—(O—CH₂—CH₂)ₖ—O—(CH₂)ₚ—C(O)—]—NH—(CH₂)_{q}—X-§
d. #—Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X-§
wherein —Z— represents independently of each other a bond or —NH—CH(COOH)—(CH₂)₂—C(O)— or —NH—CH[(CH₂)₂COOH]—C(O)—, X is independently of each other OP(O)(OH), wherein k is independently of each other an integer of 1 or 2, wherein m is independently of each other an integer of 4 to 8, wherein n is 2, and wherein p is 1, wherein q is independently of each other an integer of 1 to 6, wherein r is independently of each other an integer of 1 to 3, preferably 1 or 2, and wherein said (#) represents the point of covalent linkage to said lipid moiety and said (§ ) represents the point of covalent linkage to said oligomeric compound.

In some embodiments, the spacer comprises, preferably is, independently selected from any one of the formulae:
a. #—Z—NH—(CH₂)ₘ—X-§
b. #—Z—NH—(CH₂)ₙ—(O—CH₂—CH₂)ₖ—O—(CH₂)ₚ—X-§
c. #—Z[—NH—(CH₂)ₙ—(O—CH₂—CH₂)ₖ—O—(CH₂)ₚ—C(O)—]—NH—(CH₂)_{q}—X-§
d. #—Z[—NH—(CH₂)ₙ—(O—CH₂—CH₂)ₖ—O—(CH₂)ₚ—C(O)—]ᵣ—NH—(CH₂)_{q}—(O—CH₂—CH₂)ₖ—X-§
wherein —Z— represents independently of each other a bond or —NH—CH(COOH)—(CH₂)₂—C(O)— or —NH—C[(CH₂)₂COOH]—C(O)—, X is independently of each other OP(O)(SH) or OP(O)(OH), wherein k is 1, wherein m is 6, wherein n is 2, and wherein p is 1, wherein q is independently of each other an integer of 1 to 6, wherein r is independently of each other an integer of 1 to 3, preferably 1 or 2, wherein said (#) represents the point of covalent linkage to said lipid moiety and said (§ ) represents the point of covalent linkage to said oligomeric compound.

In some embodiments, the spacer comprises, preferably is, #—Z—NH—(CH₂)ₘ—X-§ , wherein —Z— represents a bond, X is independently of each other OP(O)(SH) or OP(O)(OH), wherein m is 6, wherein said (#) represents the point of covalent linkage to said lipid moiety and said (§ ) represents the point of covalent linkage to said oligomeric compound.

In some embodiments, the spacer comprises, preferably is, #—Z—NH—(CH₂)ₘ—X-§ , wherein —Z— represents a bond, X is OP(O)(OH), wherein m is 6, wherein said (#) represents the point of covalent linkage to said lipid moiety and said (§ ) represents the point of covalent linkage to said oligomeric compound.

In some embodiments, the spacer comprises, preferably is, #—Z—NH—(CH₂)ₘ—X-§ , wherein —Z— represents a bond, X is OP(O)(SH), wherein m is 6, wherein said (#) represents the point of covalent linkage to said lipid moiety and said (§ ) represents the point of covalent linkage to said oligomeric compound.

In some embodiments, said one or more lipid moiety is covalently linked to said oligomeric compound either directly or via a spacer through a —OP(O)(SH)— or a —OP(O)(OH)—moiety, typically and preferably comprised by said one or more lipid moiety or said spacer, wherein said —OP(O)(SH)— or said —OP(O)(OH)— moiety is linked to the 5′-terminal OH-group or to the 3′-terminal OH-group of said oligomeric compound.

In some embodiments, said one or more lipid moiety is independently of each other linked to said oligomeric compound at (i) a terminal residue of said oligomeric compound, (ii) the 5′ terminus of said oligomeric compound, (iii) the 3′ terminus of said oligomeric compound; (iv) an internal residue of said oligomeric compound.

In some embodiments, said one or more lipid moiety, preferably said exactly one lipid moiety, is independently of each other linked to said oligomeric compound at a terminal residue of said oligomeric compound.

In some embodiments, said one or more lipid moiety, preferably said exactly one lipid moiety, is independently of each other linked to said oligomeric compound at the 5′ terminus of said oligomeric compound.

In some embodiments, said one or more lipid moiety, preferably said exactly one lipid moiety, is independently of each other linked to said oligomeric compound at the 3′ terminus of said oligomeric compound.

In some embodiments, said one or more lipid moiety, preferably said exactly one lipid moiety, is independently of each other linked to said oligomeric compound at an internal residue of said oligomeric compound.

In some embodiments, said one or more lipid moiety, preferably exactly one lipid moiety, is covalently linked to said oligomeric compound, preferably to said oligonucleotide, either directly or via a spacer through a —OP(O)(SH)— or a —OP(O)(OH)— or a —NHP(O)(OH)— or a —NHP(O)(SH)— or a —NH—C(O)— moiety, typically and preferably comprised by said one or more lipid moiety or said spacer, wherein said —OP(O)(SH)— or said —OP(O)(OH)— or said —NHP(O)(OH)— or said —NHP(O)(SH)— or said —NH—C(O)— moiety is linked to the 5′-terminal OH-group or to the 3′-terminal OH-group of said oligomeric compound.

In some embodiments, said one or more lipid moiety, preferably exactly one lipid moiety, is covalently linked to said oligomeric compound, preferably to said oligonucleotide, either directly or via a spacer through a —OP(O)(SH)— or a —OP(O)(OH)—moiety, wherein said —OP(O)(SH)— or said —OP(O)(OH)— moiety is linked to the 5′-terminal OH-group or to the 3′-terminal OH-group of said oligomeric compound, and wherein typically and preferably said —OP(O)(SH)— or said —OP(O)(OH)— moiety is comprised by said one or more lipid moiety or said spacer.

In some embodiments, said one or more lipid moiety, preferably exactly one lipid moiety, is covalently linked to said oligomeric compound, preferably to said oligonucleotide, either directly or via a spacer through a —OP(O)(SH)— moiety, wherein said —OP(O)(SH)— moiety is linked to the 5′-terminal OH-group or to the 3′-terminal OH-group of said oligomeric compound, and wherein typically and preferably said —OP(O)(SH)— moiety is comprised by said one or more lipid moiety or said spacer.

In some embodiments, said one or more lipid moiety, preferably exactly one lipid moiety, is covalently linked to said oligomeric compound, preferably to said oligonucleotide, either directly or via a spacer through a —OP(O)(SH)— moiety, wherein said —OP(O)(SH)— moiety is linked to the 5′-terminal OH-group of said oligomeric compound, and wherein typically and preferably said —OP(O)(SH)— moiety is comprised by said one or more lipid moiety or said spacer.

In some embodiments, said one or more lipid moiety, preferably exactly one lipid moiety, is covalently linked to said oligomeric compound, preferably to said oligonucleotide, either directly or via a spacer through a —OP(O)(SH)— moiety, wherein said —OP(O)(SH)— moiety is linked to the 3′-terminal OH-group of said oligomeric compound, and wherein typically and preferably said —OP(O)(SH)— moiety is comprised by said one or more lipid moiety or said spacer.

In some embodiments, said one or more lipid moiety, preferably exactly one lipid moiety, is covalently linked to said oligomeric compound, preferably to said oligonucleotide, either directly or via a spacer through a —OP(O)(OH)— moiety, wherein said —P(O)(OH)— moiety is linked to the 5′-terminal OH-group or to the 3′-terminal OH-group of said oligomeric compound, and wherein typically and preferably said —OP(O)(OH)— moiety is comprised by said one or more lipid moiety or said spacer.

In some embodiments, said one or more lipid moiety, preferably exactly one lipid moiety, is covalently linked to said oligomeric compound, preferably to said oligonucleotide, either directly or via a spacer through a —OP(O)(OH)— moiety, wherein said —P(O)(OH)— moiety is linked to the 5′-terminal OH-group of said oligomeric compound, and wherein typically and preferably said —OP(O)(OH)— moiety is comprised by said one or more lipid moiety or said spacer.

In some embodiments, said one or more lipid moiety, preferably exactly one lipid moiety, is covalently linked to said oligomeric compound, preferably to said oligonucleotide, either directly or via a spacer through a —OP(O)(OH)— moiety, wherein said —P(O)(OH)— moiety is linked to the 3′-terminal OH-group of said oligomeric compound, and wherein typically and preferably said —OP(O)(OH)— moiety is comprised by said one or more lipid moiety or said spacer.

In a particular embodiment, at least one lipid moiety linked to the oligomeric compound is a saturated fatty acid moiety, more particularly a saturated fatty acid moiety derived from palmitic acid (C16) or stearic acid (C18) and most particularly a saturated fatty acid moiety derived from palmitic acid (C16). In some embodiments, the at least one lipid moiety comprises palmitic acid (C16).

In the present invention, at least one lipid moiety is linked to the oligomeric compound at (i) a terminal residue of said oligomeric compound, (ii) the 5′-terminus of said oligomeric compound, (iii) the 3-terminus of said oligomeric compound; or (iv) an internal residue of said oligomeric compound.

When the linkage between the oligomeric compound and at least one lipid moiety is direct, the covalent link implies one atom of the oligomeric compound and one atom of the at least one lipid moiety.

When the linkage between the oligomeric compound and the at least one lipid moiety is indirect, there is a spacer. One first atom of this spacer is covalently linked to one atom of the oligomeric compound while a second atom of this spacer different from the first one is covalently linked to one atom of the at least one lipid moiety.

Any spacer disclosed can be implemented in the present invention. In a particular embodiment, the spacer implemented in the invention is of below formula (A) or (B):
ø—NH—C₂₋₁₂alkylene-OP(═O)(OH)-§ (A)
ø—NH—C₂₋₁₂alkylene-OP(═S)(OH)-§ (B),
in which (ø) represents the point of covalent linkage to the lipid moiety and (§ ) represents the point of covalent linkage to the oligomeric compound.

It should be noted that the spacer of formula (B) is in equilibrium with the spacer of below formula (B′):
ø—NH—C₂₋₁₂alkylene-OP(SH)(═O)-§ (B′).

Thus formula (B) and formula (B′) are equivalent and can be used interchangeably.

In a more particular embodiment, the spacer is of formula (B) and advantageously the alkylene chain this spacer is 6 carbon atoms long.

In some embodiments, the oligomeric compound according to the present invention is selected from the group consisting of:
C₈₋₂₆-saturated fatty acid moiety-NH—C₂₋₁₂alkylene-OP(═S)(OH)—GGAGATgGCAGTTTC-3′ (SEQ ID NO: 4 in the appended sequence listing),
C8-26-saturated fatty acid moiety-NH—C₂₋₁₂alkylene-OP(═S)(OH)—GGAGATGgCAGTTTC-3′ (SEQ ID NO: 5 in the appended sequence listing),
C8-26-saturated fatty acid moiety-NH—C₂₋₁₂alkylene-OP(═S)(OH)—GGAGATGGcAGTTTC-3′ (SEQ ID NO: 6 in the appended sequence listing), and
C8-26-saturated fatty acid moiety-NH—C₂₋₁₂alkylene-OP(═S)(OH)—GGAGATGGCaGTTTC-3′ (SEQ ID NO: 7 in the appended sequence listing).

In some embodiments, the oligomeric compound according to the present invention is selected from the group consisting of:
palmitate-NH—C₂₋₁₂alkylene-OP(═S)(OH)-GGAGATgGCAGTTTC-3′ (SEQ ID NO: 4 in the appended sequence listing),
palmitate-NH—C₂₋₁₂alkylene-OP(═S)(OH)-GGAGATGgCAGTTTC-3′ (SEQ ID NO: 5 in the appended sequence listing),
palmitate-NH—C₂₋₁₂alkylene-OP(═S)(OH)-GGAGATGGcAGTTTC-3′ (SEQ ID NO: 6 in the appended sequence listing), and
palmitate-NH—C₂₋₁₂alkylene-OP(═S)(OH)-GGAGATGGCaGTTTC-3′ (SEQ ID NO: 7 in the appended sequence listing).

In some embodiments, the oligomeric compound according to the present disclosure is selected from the group consisting of:
C₈₋₂₆-saturated fatty acid moiety-NH—C₆alkylene-OP(═S)(OH)—GGAGATgGCAGTTTC-3′ (SEQ ID NO: 4 in the appended sequence listing),
C₈₋₂₆-saturated fatty acid moiety-NH—C₆alkylene-OP(═S)(OH)—GGAGATGgCAGTTTC-3′ (SEQ ID NO: 5 in the appended sequence listing),
C₈₋₂₆-saturated fatty acid moiety-NH—C₆alkylene-OP(═S)(OH)—GGAGATGGcAGTTTC-3′ (SEQ ID NO: 6 in the appended sequence listing), and
C₈₋₂₆-saturated fatty acid moiety-NH—C₆alkylene-OP(═S)(OH)—GGAGATGGCaGTTTC-3′ (SEQ ID NO: 7 in the appended sequence listing).

As particular examples of the oligomeric compound according to the present invention, one can cite:
palmitate-NH—C6alkylene-OP(═S)(OH)-REGONE.7,
palmitate-NH—C6alkylene-OP(═S)(OH)-REGONE.8 (hereinafter designed as SKY51),
palmitate-NH—C6alkylene-OP(═S)(OH)-REGONE.9, and
palmitate-NH—C6alkylene-OP(═S)(OH)-REGONE.10.

In other words, the oligomeric compound according to the present invention is selected from the group consisting of:
palmitate-NH—C₆alkylene-OP(═S)(OH)-GGAGATgGCAGTTTC-3′ (SEQ ID NO: 4 in the appended sequence listing),
palmitate-NH—C6alkylene-OP(═S)(OH)-GGAGATGgCAGTTTC-3′ (SEQ ID NO: 5 in the appended sequence listing),
palmitate-NH—C6alkylene-OP(═S)(OH)-GGAGATGGcAGTTTC-3′ (SEQ ID NO: 6 in the appended sequence listing), and
palmitate-NH—C6alkylene-OP(═S)(OH)-GGAGATGGCaGTTTC-3′ (SEQ ID NO: 7 in the appended sequence listing).

In some embodiments, the oligomeric compound is palmitate-NH—C6alkylene-OP(═S)(OH)-GGAGATgGCAGTTTC-3′ (SEQ ID NO: 4 in the appended sequence listing).

In some embodiments, the oligomeric compound is palmitate-NH—C₆alkylene-OP(═S)(OH)-GGAGATGgCAGTTTC-3′ (SEQ ID NO: 5 in the appended sequence listing).

In some embodiments, the oligomeric compound is palmitate-NH—C₆alkylene-OP(═S)(OH)-GGAGATGGcAGTTTC-3′ (SEQ ID NO: 6 in the appended sequence listing).

In some embodiments, the oligomeric compound is palmitate-NH—C₆alkylene-OP(═S)(OH)-GGAGATGGCaGTTTC-3′ (SEQ ID NO: 7 in the appended sequence listing).

The present invention concerns an oligomeric compound as previously defined for use as a medicament. Thus the present invention relates to a pharmaceutical composition comprising, as an active ingredient, an oligomeric compound according to the present invention and a pharmaceutically acceptable vehicle. In some embodiments, the pharmaceutical composition comprises a therapeutically effective amount of an oligomeric compound described herein.

The pharmaceutical compositions according to the invention can be employed by the systemic route; by the parenteral route, for example the intravenous, intra-arterial, intraperitoneal, intrathecal, intra-ventricular, intrasternal, intracranial, intramuscular or sub-cutaneous route; by topical route; by the oral route; by the rectal route; by the intranasal route or by inhalation.

As solid compositions for oral administration, tablets, pills, powders, etc. can be used where the oligomeric compound according to the invention is mixed with one or more conventionally used inert diluents, and possibly other substances such as, for example, a lubricant, a colorant, a coating etc.

As liquid compositions for oral or ocular administration pharmaceutically acceptable, suspensions, solutions, emulsions, syrups containing conventionally used inert diluents, and possibly other substances such as wetting products, sweeteners, thickeners, etc. can be used.

The sterile compositions for parenteral administration can be aqueous or non-aqueous (oleaginous) solutions, suspensions or emulsions. As a solvent or vehicle, water, propylene-glycol, plant oils or other suitable organic solvents can be used. These compositions can also contain adjuvants, such as dispensing or wetting agents, suspending agents, isotonisers, emulsifiers, etc.

The compositions for topic administration can be for example creams, lotions, oral sprays, nose or eye drops or aerosol.

Those skilled in the art will recognize that the amount of an oligomeric compound to be administered will be an amount that is sufficient to induce amelioration of unwanted disease symptoms. Such an amount may vary inter alia depending on such factors as the age, weight, overall physical condition, of the patient, etc. and may be determined on a case by case basis. The amount may also vary according to the other components of a treatment protocol (e.g., administration of other medicaments such as steroids, etc.). Those skilled in the art will recognize that such parameters are normally worked out during clinical trials. Further, those skilled in the art will recognize that, while disease symptoms may be completely alleviated by the treatments described herein, this is not an absolute requirement. Even a partial or intermittent relief of symptoms may be of great benefit to the recipient. In addition, treatment of the patient is usually not a single event. Rather, the oligomeric compounds will likely be administered on multiple occasions, that may be, depending on the results obtained, several days apart, several weeks apart, or several months apart, or even several years apart.

The present invention also concerns an oligomeric compound as previously defined or a pharmaceutical composition as previously defined for use in treating Duchenne Muscular Dystrophy in a patient in need.

In another aspect, the disclosure includes a method for treating Duchenne Muscular Dystrophy in a patient in need. In some embodiments, the method comprises administering to the patient a therapeutically effective dose of the oligomeric compound disclosed herein or the pharmaceutical compositions disclosed herein.

It is understood in the art that splice-switching strategies can be used for the treatment of patients with DMD disease. In particular, a significant subset of patients with DMD, corresponding to those having large deletions taking away one or several exons such as Δ43-50, Δ45-50, Δ47-50, Δ48-50, Δ49-50, Δ50, Δ52 or Δ52-58, could potentially benefit from the present invention aiming to realize skipping of exon 51, although clinical benefit for each patient will depend on the quality of the truncated dystrophin generated from his specific genetic deletion.

Those skilled in the art will recognize that there are many ways to determine or measure a level of efficacy in response to a treatment such as splice switching. Such methods include but are not limited to measuring or detecting an activity of the rescued protein in patient cells or in appropriate animal models. It is also possible to gauge the efficacy of a treatment protocol intended to modify the exon composition of a mRNA by using RT-PCR for assessing the presence of the targeted exon in patient cells as well as in normal cells or in wild type animal models if interspecies homology permits.

Other features and advantages of the present invention will become apparent from the following detailed description which makes reference to the accompanying drawings.

### BRIEF DESCRIPTION OF THE DRAWINGS

FIG. 1 illustrates the sequence REGONE (SEQ ID NO:3). FIG. 1A shows the very shape of the REGONE sequence with full tc-DNA constrained sugar backbone -3D modeling was achieved by using a set of appropriate computer tools and published experimental data obtained from NMR, CD spectroscopic structural investigations and crystal structure of tc-DNA. FIG. 1B shows the partial pairing of the REGONE sequence with itself -The symbol “I” refers to possible Watson-Crick pairing -If it were an oligomer made up of canonical DNA, this structure would be unstable (Tm below 15° C.). However, 3D modeling of these dimers suggests that such forms may exist in the presence of a preorganized constrained backbone such as tc-DNA-“G-C” pairings are indicated in black, “A=T” parings are in dark grey, nucleotides not involved in base pairing are in soft grey.

FIG. 2 illustrates the outcome on the overall shape of the REGONE tc-DNA-based oligonucleotide after changing a single tc-DNA nucleotide by an equivalent nucleotide with 2OMe-ribose sugar. FIG. 2A shows the putative effect of such modification at position 8 on the 3D shape —Subsequent oligomer is named “REGONE.8” (SEQ ID NO:5). The 2′OMe-nucleotide introduces a point of flexion within the tc-DNA string, which disrupts its overall preorganized structure, thus abolishing its capacity to sustain dimeric forms raising from incomplete base-pairing. FIG. 2B shows gel electrophoresis experiments in non-denaturing conditions demonstrating that REGONE.8 is indeed no longer able to form dimers. The same sequence consisting only of tcDNA nucleotides systematically presents dimerized forms migrating differently in the gel (note that the proportion of dimerized forms may be higher).

FIG. 3 illustrates the composition of the compound SQY51. FIG. 3A shows that it comprises the antisense oligonucleotide REGONE.8 (SEQ ID NO:5) covalently attached to a palmitoyl residue at its 5′ end via a C6 linker. The Chemical Formula of SQY51 is C₂₁₅H₂₆₃N₆₀O₉₃P₁₅S; Exact Mass 5669.35; Molecular Weight 5672.46. FIG. 3B shows gel electrophoresis experiments in non-denaturing conditions demonstrating that the addition of the palmitoyl residue has no effect on the dimerization properties of the oligonucleotides. SQY51 is still not able to form dimers while its counterpart with full tc-DNA nucleotides does.

FIG. 4 illustrates the results of a complement assay using human serum for SQY51 and the same compound with full phosphorothioate internucleoside linkages (SQY51-PS). Phosphate buffered saline (PBS) was used as negative control for complement activation, whereas Zymosan, a glucan with repeating glucose units connected by 3-1,3-glycosidic linkages found on the surface of yeast, was used as positive control. The experimental drug concentration for in vitro testing of complement activation was 2 mg/mL, which mimics an in vivo dose-regimen of about 150 mg/kg with a theoretical Cₘₐₓ of about 0.4 mM (likely much more since the volume of blood as plasma was considered in this extrapolation).

FIG. 5 illustrates the results of clotting assays using human plasma for SQY51 and the same compound with full phosphorothioate internucleoside linkages (SQY51-PS). Phosphate buffered saline (PBS) was used as control. The Prothrombin Time (PT, FIG. 5A) and the Activated Partial Thromboplastin Time (APTT, FIG. 5B) are two blood-tests that measure how long it takes for blood to clot in the presence of an experimental drug.

FIG. 6 illustrates: FIG. 6A: SDS-PAGE analysis of protein recovery from human, macaque and mouse sera using a biotinylated-SQY51 molecule (the biotin moiety was attached at the 3′ end of the oligonucleotide depicted in FIG. 3 through a C3 linker —not shown). FIG. 6B: 3D-modeling of human albumin and mouse albumin interacting with SQY51. Albumin is represented as “ribbons”, the palmitoyl-C₆-amino in “real volume”, and REGONE.8 as “batons”. FIG. 6C: SDS-PAGE analysis of protein recovery from Human, macaque and mouse sera using different biotinylated compounds such as SYN51 (Palmitoyl-amino-C₆-SEQ ID NO: 8), M23D (Palmitoyl-amino-C₆-SEQ ID NO: 9), and SQY51 (Palmitoyl-amino-C₆-SEQ ID NO: 5); a biotin moiety was attached at the 3′ end of each oligonucleotide through a C3 linker.

FIG. 7 illustrates determination of blood pharmacokinetic (PK) parameters of SQY51 after intravenous infusion (dose of 50 mg/kg -30 min infusion) in cynomolgus monkey (Macaca fascicularis). Typically, determination of PK parameters requires a number of blood samples taken at different time points to estimate maximum concentration in plasma (Cmax), half-life (t %), volume of distribution (VD) and area under the curve (AUC). FIG. 7A shows plasma concentration of SQY51 over a week after infusion. FIG. 7B shows time profile deduced from a two-compartment model (Phoenix WinNonlin 8.1). Plasma concentration (Cp) of SQY51 follows a bi-exponential kinetics as previously described for other sorts of oligonucleotides. PK parameters for SQY51 are: Cmax=900±120 μg/mL; αt½=2±0.3 h; βt½=90±3 h.

FIG. 8 illustrates the results of complement activation in cynomolgus monkey (Macaca fascicularis) at different time points (pretest, 10′, 30′, 1h, 2h, 4h, 8h, 24h and 48h) after systemic delivery of SQY51 via an intravenous infusion using a dose of 50 mg/kg in phosphate buffered saline (PBS). PBS alone and a corresponding compound with full phosphorothioate internucleoside linkages (SQY51-PS) were used as negative and positive controls, respectively. FIG. 8A shows the progression of C3a levels, which mirror the C3 consumption. FIG. 8B shows the progression of Bb, a proteolytic enzyme, which is generated by the cleavage of the factor B during the activation of the alternative pathway (AP) of the complement system. FIG. 8C shows the development of SC5b-9, a soluble complex of S Protein and C5b-9 resulting from the activation of complement in the absence of bilipid layer membranes.

FIG. 9 illustrates the results of clotting assays in blood samples from cynomolgus monkey (Macaca fascicularis) at different time points (pretest, 30′, 4h and 24h) after systemic delivery of SQY51 via an intravenous infusion using a dose of 50 mg/kg in phosphate buffered saline (PBS). PBS alone and a corresponding compound with full phosphorothioate internucleoside linkages (SQY51-PS) were used as negative and positive controls, respectively. The Prothrombin Time (PT, FIG. 9A) and the Activated Partial Thromboplastin Time (APTT, FIG. 9B) are two blood-tests that measure how long it takes for blood to clot in the presence of an experimental drug.

FIG. 10 illustrates cytokines levels in blood samples over 48h post-infusion after a single dose of SQY51 (50 mg/kg -30 min infusion). The results obtained with SYN51-PS are superimposed to exemplify the consequences of a compound triggering a toxic response. Are disclosed the levels of Interleukin 1 beta (IL-1S) (FIG. 10A), the levels of Interleukin 6 (IL-6) (FIG. 10B), the levels of Monocyte Chemoattractant Protein 1 (MCP-1) (FIG. 10C) and the levels of Tumor Necrosis Factor alfa (TNF-α) (FIG. 10D).

FIG. 11 illustrates biodistribution of SQY51 in monkey tissues after one week or five weeks after the end of a 4-week treatment with a dosage of 50 mg/kg/week; amounts of compounds were measured with the beacon method. Graph shows superimposed results for SQY51 quantification at one week after the end of the treatment (white plots) and after 4 additional weeks (black plots). The % indicate the rate of clearance after 4 weeks of washout.

FIG. 12 illustrates levels of exon 51-skipped dystrophin mRNA in monkey tissues. FIG. 12A exemplifies detection of exon-51 skipping using nested RT-PCR in gastrocnemius, quadriceps, deltoid, biceps, diaphragm, heart, cerebellum, spinal cord, skin, stomach, duodenum and ileum, one week after 4 weekly injections of SQY51 with a dosage of 50 mg/kg/week. Reverse-transcribed mRNAs were sequentially amplified with PCR1 (Ex46F/Ex53R) and PCR2 (Ex47Fi/Ex52Ri) in order to picture an amplicon of 886 base-pairs (bp) rising from normally spliced dystrophin mRNAs, and an amplicon of 653 bp missing exon-51 as a result of the effect of SQY51. FIG. 12B shows the percentage of remaining skipping levels in striated muscles at 5 weeks post-treatment (black plots) comparatively to levels at one week after last injection (the background display in grey refers to the levels attained at one-week post-treatment for each muscle; n=4 animals per group).

FIG. 13 illustrates a comparison between biodistribution of SQY51 and SYN51 in monkey tissues one week after a 4-week treatment with a dosage of 50 mg/kg/week; amounts of compounds were measured using LC-MS/MS. Graph shows superimposed results for SQY51 quantification (white plots) and SYN51 (black plots).

### DETAILED DISCUSSION OF PARTICULAR EMBODIMENTS

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Compounds for the Treatment of Duchenne Muscular Dystrophy

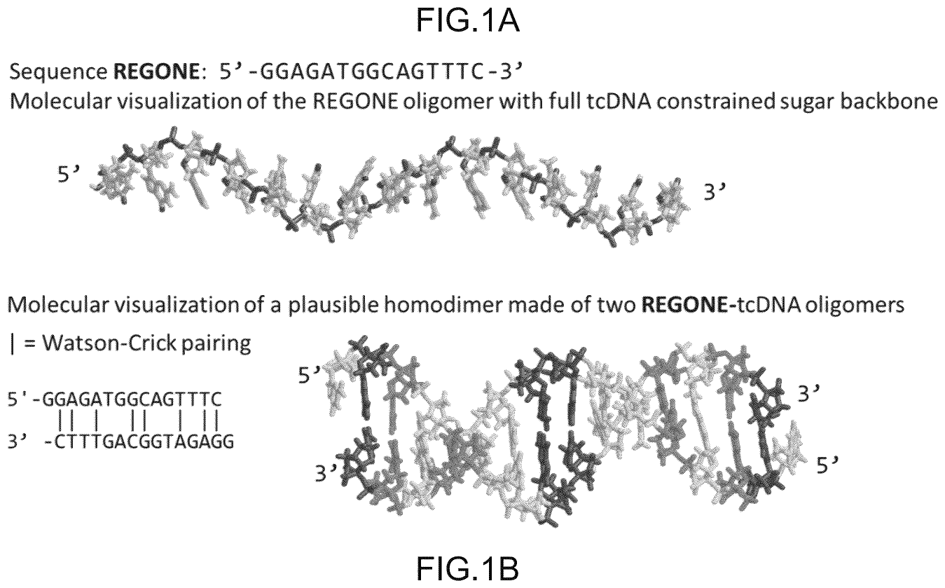

Typically, small antisense oligonucleotides involve the use of nucleic acids whose skeleton is made more rigid by means of a constrained sugar backbone, as it is the case for LNAs (locked nucleic acids) or tricyclo-DNA as examples. FIG. 1 illustrates the REGONE sequence (SEQ ID NO:3) made of tricyclo-DNA nucleotides, whose constrains on ribose-moieties impose an overall shape to the oligomer so that its Tm is increased by 2-3° C. per nucleotide. As a reminder, the melting temperature (Tm) of an oligonucleotide or oligomeric compound refers to the temperature at which 50% of the oligonucleotide is in a duplex with its complement. The counterpart of this preconfigured rigidity is that a sequence not supposed to form homodimers could still do so and therefore prove to be highly toxic in vivo, in particular when the internucleotide links are of phosphorothioate (PS) type to improve biodistribution (Echevarria et al, Nucleic Acid Ther., 2019, vol. 99, pages 148-160; Aupy et al, Mol Ther Nucleic Acids., 2019, vol. 19, pages 371-383).

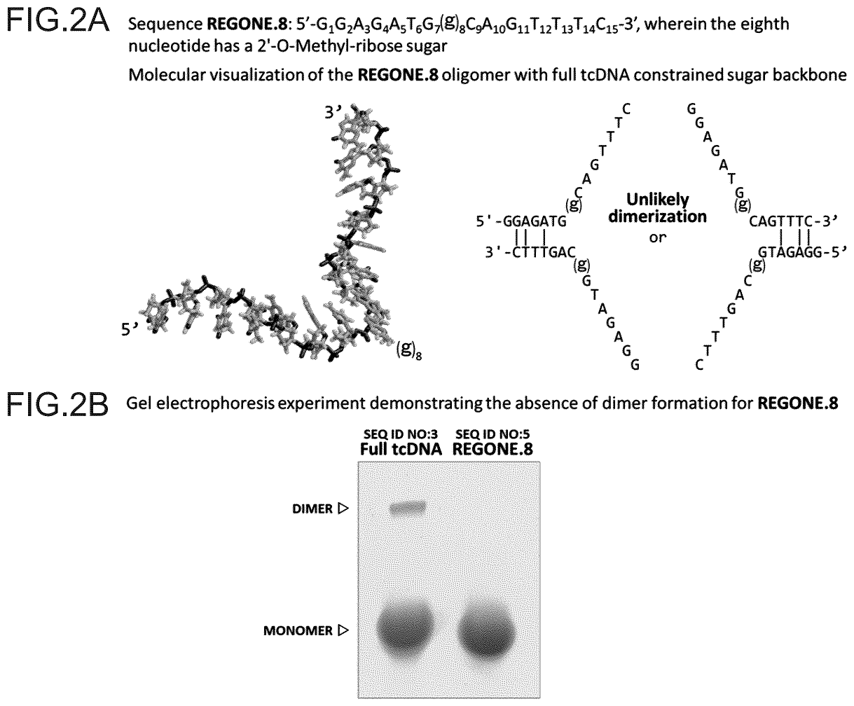

The tendency of REGONE to homodimerize was solved by introducing an unconstrained nucleotide within the sequence. As illustrated in FIG. 2, such modification upsets the very organization of the tricyclo-DNA oligomer, which has now an inner degree of flexibility so that an imperfect pairing will not be maintained. This is confirmed by (i) 3D-modeling of a REGONE variant where the eighth tc-DNA nucleotide is replaced by an equivalent 2′-O-Methyl-ribose RNA (so called REGONE.8), and (ii) in gel electrophoresis experiments demonstrating that subsequent compound is no longer able to form homodimers. Maximal effect was obtained for modification at nucleotide number-8 and −9, to less extent at nucleotide number-7 and −10, and not at all at other positions. Thus the modification should be located between nucleotides 7 and 10-REGONE.7 (SEQ ID NO: 4), REGONE.8 (SEQ ID NO: 5), REGONE.9 (SEQ ID NO: 6) and REGONE.10 (SEQ ID NO: 7). Other modifications —at position 1, 2, 3, 4, 5, 6, 11, 12, 13, 14 or 15-do not satisfactorily affect the overall preorganized structure of the molecule, which remains capable to tolerate dimer formation.

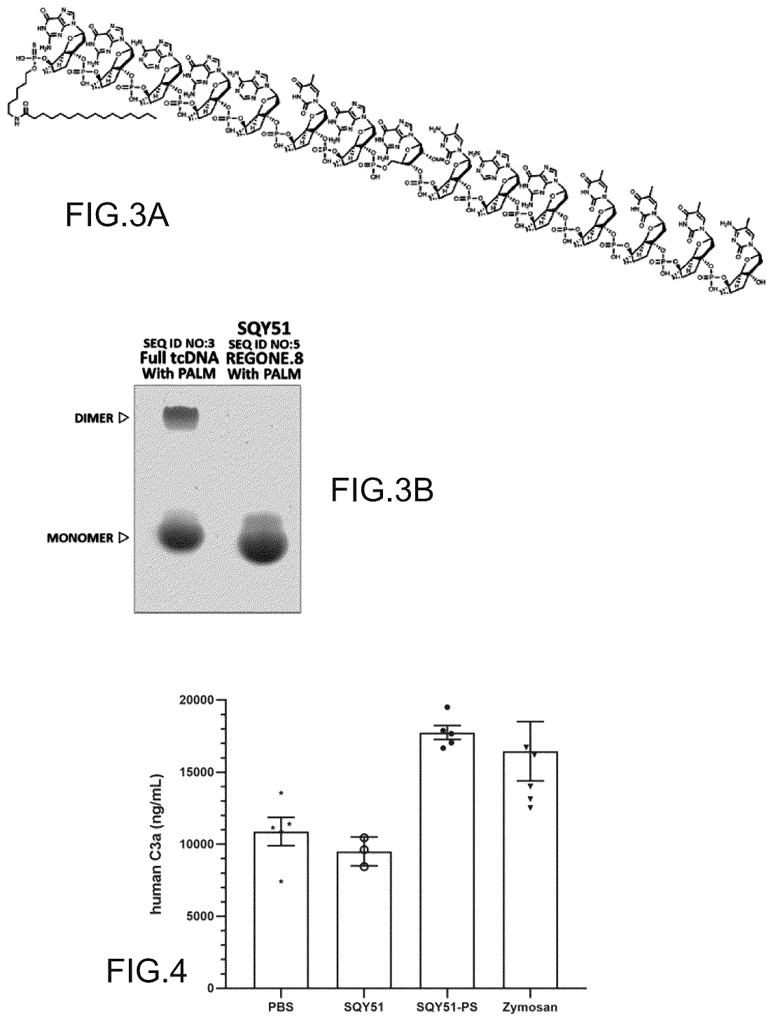

In order to guarantee, or at least not compromise, the safety of REGONE.8 under intravenous injection conditions, it was preferred to link the 15 nucleotides of the oligomer by phosphodiester (PO) bonds and not phosphorothioate (PS) like those skilled in the art would advocate to satisfy an effective biodistribution. Furthermore, the oligomers of the tricyclo-DNA class are satisfactorily stable in biological fluids and therefore do not require this modification (i.e., PS-bonds) which, on the contrary, could prove to be disadvantageous if the compound were no longer biodegradable. Finally, the REGONE.8 (SEQ ID NO: 5) was covalently attached to a palmitoyl residue at its 5′ end via a C₆ linker as shown in FIG. 3. This ultimate compound (C₂₁₅H₂₆₃N₆₀O₉₃P₁₅S) is named SQY51.

### Example 2: In Vitro Toxicity Assessment on Human Blood

Advantageously, SQY51 does not activate complement in human serum, while the same compound with phosphorothioate internucleoside linkages (SQY51-PS) does (FIG. 4); especially considering that the experimental drug concentration for such in vitro assay was 2 mg/mL, which mimics an in vivo dose-regimen of about 150 mg/kg with a theoretical Cₘₐₓ of about 0.4 mM —likely much more as the volume of blood as plasma was considered for this extrapolation.

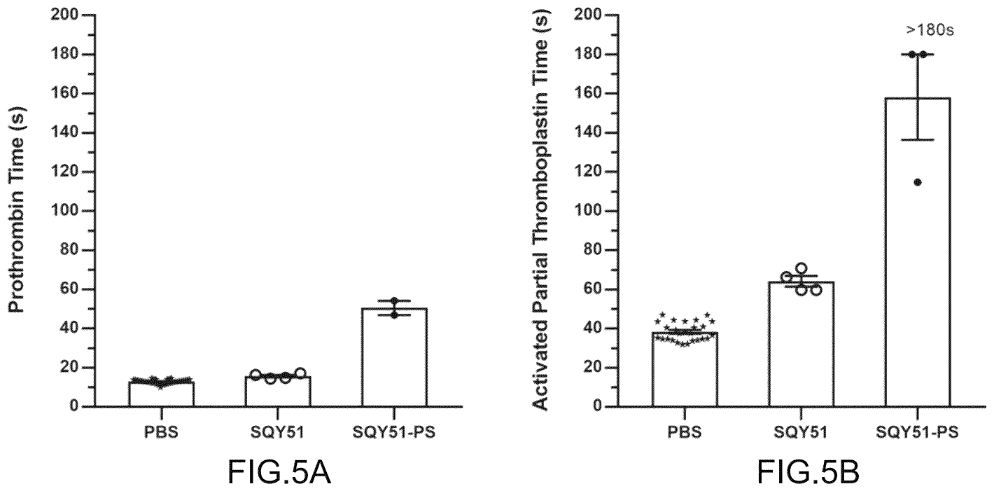

Another feared problem during intravenous injections of AONs is their possible interaction with the coagulation system. Several routine tests exist to assess the effect of candidate drugs on coagulation such as Prothrombin Time (PT) and Activated Partial Thromboplastin Time (APTT). The latter are two blood-tests that measure how long it takes for blood to clot in the presence of an experimental drug). Prolongation of clotting times (PT & APTT) indicates an anticoagulant effect (risk of excessive bleeding), while shortening indicates a procoagulant effect (risk of developing clots —thrombosis). The experimental drug concentration for clotting assays was 2 mg/mL, which mimics an in vivo dose-regimen of about 150 mg/kg with a theoretical Cₘₐₓ of about 0.4 mM (likely much more since the volume of blood as plasma was considered in this extrapolation). FIG. 5 clearly shows that SQY51 does not significantly modify clotting parameters in human plasma, while SQY51-PS does, particularly by increasing the clotting time of the extrinsic coagulation pathway (APTT).

### Example 3: Oligo-Captured Serum Proteins

A further important subject is which experimental model to use to validate an innovation in the field of AONs. Most often, the skilled person merely established the efficacy of a novel AON on appropriate cells in tissue culture then perhaps in vivo using murine models. Unfortunately, such approach presupposes that the antisense compound will interact with the body fluids in the same way regardless of the species, a postulate which must be confirmed. To this end, it was investigated which proteins in the serum were capable of binding to SQY51. While most of the sequences tested (i.e., with same type of design) gave very similar capture profiles, it is remarkable that SQY51 is unique because it presents major differences between species.

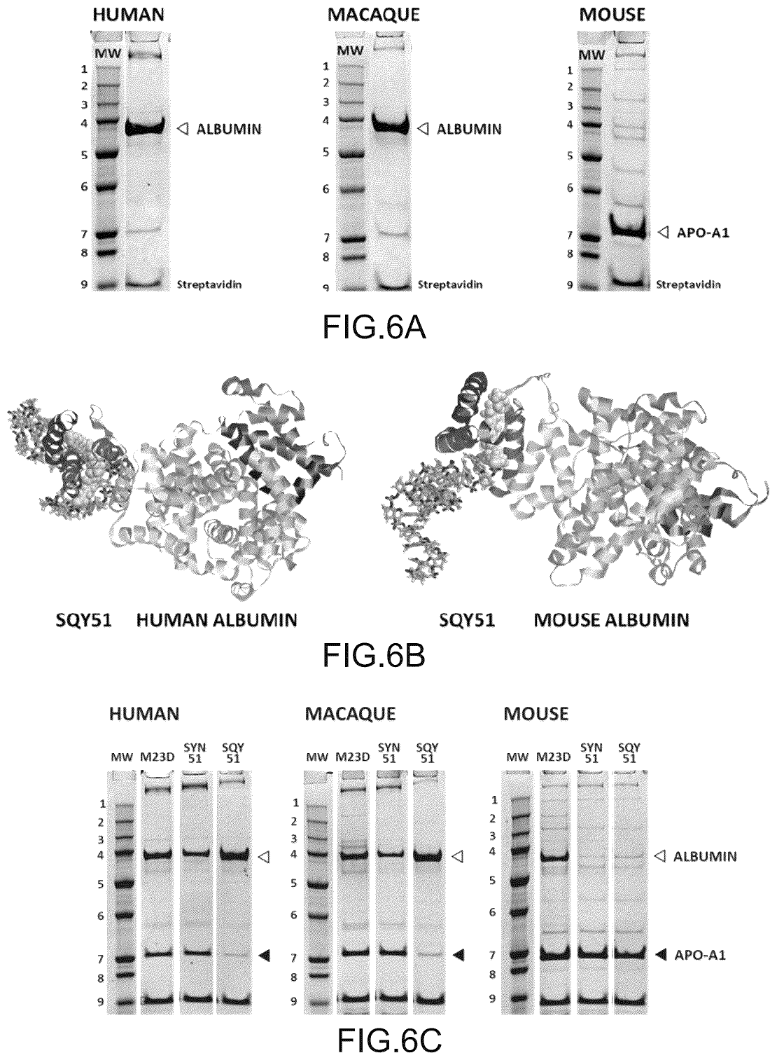

SQY51 molecule preferentially retains serum albumin in the human and the macaque sera whereas it is APO-A1 in the mouse serum (FIG. 6A).

Molecular dynamic studies with GROMACS (Abraham et al, SoftwareX, 2015, vol. 1-2, pages 19-25) indicate that SQY51 can interact with the mouse albumin only through its palmitoyl moiety (FIG. 6B). In agreement with experimental data shown in FIG. 6A, the 3D-analysis shows that SQY51 interacts more strongly with the human albumin; here, in addition to the interaction with the palmitoyl residue, the REGONE.8 moiety can also interact with the protein to strengthen the assembly.

FIG. 6C discloses the SDS-PAGE analysis of protein recovery from human, macaque and mouse sera using different biotinylated compounds such as SYN51 (Palmitoyl-amino-C₆-SEQ ID NO: 8), M23D (Palmitoyl-amino-C6-SEQ ID NO: 9), and SQY51 (Palmitoyl-amino-C₆-SEQ ID NO: 5); a biotin moiety was attached at the 3′ end of each oligonucleotide through a C3 linker. The sequence SEQ ID NO: 8 in the appended sequence listing reads: 5′-AAGAuGGCATTTCTA-3′ and the sequence SEQ ID NO: 9 in the appended sequence listing reads: 5′-CCTCGGCTTACCT-3′ in which tcDNA nucleosides are typed in capital letters while the 2′OMe-nucleoside is typed in lowercase letter.

Note that M23D is a full tc-DNA oligomer, while SYN51 and SQY51 both comprise a within the tc-DNA chain. M23D captured a similar pattern of proteins independently of tested species (albumin and apolipoproteins), which is similar to that of SYN51 in Human and macaque. Only SQY51 has the characteristic of preferentially fixing albumin in primate sera whether of human or non-human origin.

As shown in FIG. 6, SQY51 preferentially retains serum albumin in human and non-human primate sera while it interacts predominantly with apolipoproteins in mouse. 3D-modeling of SQY51 with human and mouse albumin confirmed that both proteins could take over the palmitoyl residue. Nonetheless, it appeared also that the REGONE.8 moiety of SQY51 could itself interact with the human albumin to strengthen the assembly, something that does not take place with the mouse albumin. This species-specific performance of the SQY51 is unique and was obviously not foreseeable by those skilled in the art. It also indicates that it is essential to further assess the advantages of this compound in a suitable animal model (e.g., non-human primate) such as cynomolgus monkey.

### Example 4: Pharmacokinetics and Evaluation of Toxicity of the Compounds SQY51(Present Invention) and SYN51(Prior Art) in Cynomolgus Monkey

First of all, the pharmacokinetics of SQY51 were compared to those of SYN51, also comprising an oligomeric 15-mer sequence having tricyclo-DNA nucleosides and a 2′-O-modified-RNA nucleoside (SEQ ID NO: 8) and a lipid moiety.

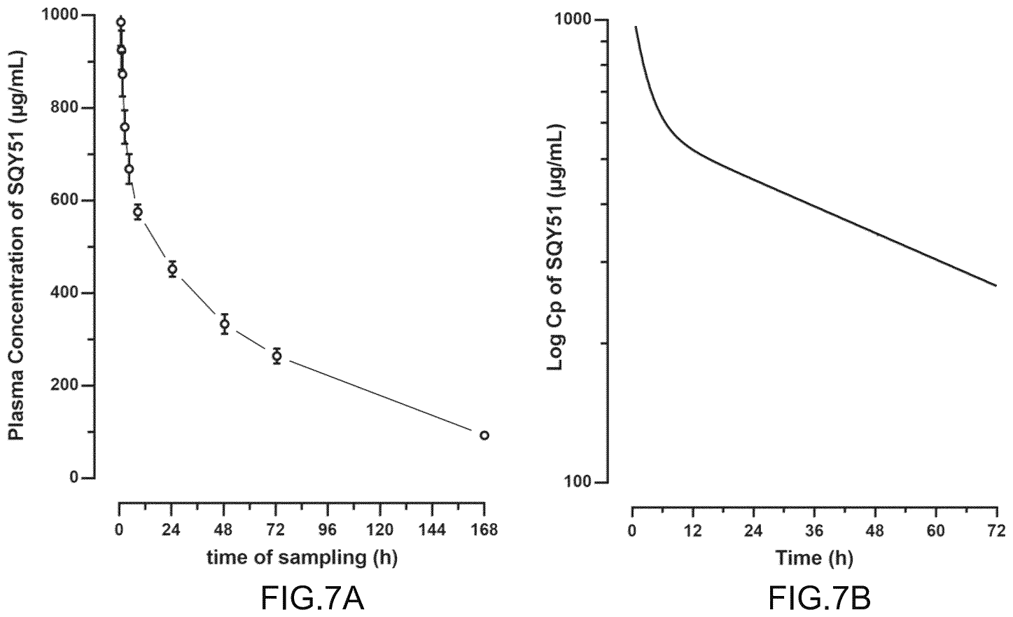

It is noteworthy in FIG. 7A that SQY51 is stable in the blood compartment (e.g., mass spectrometry analysis reveals that SQY51 remained mostly intact in plasma samples throughout the kinetic study —not shown —although a minor form, lacking the palmitoyl residue and culminating at about 0.1% of Cmax, could be detected at 24h after infusion).

Importantly, contrasting with SQY51, serum-protein capture by SYN51 showed parallel set of proteins regardless of the species (e.g., serum albumin and apolipoproteins). This difference in affinity for blood proteins was reflected in pharmacokinetic profiles. Indeed, although the two compounds follow a bi-exponential decay after intravenous infusion (50 mg/kg), their secondary PK parameters in blood are different; a 2-compartmental analysis (2C model) gives an elimination half-life of about 20 hours for SYN51 while it is about 5 times longer for SQY51 (FIG. 7).

The below Table 1 shows comparison of secondary PK parameters for SQY51 and SYN51 after a single (50 mg/kg) intravenous infusion in cynomolgus monkeys. Amounts of SQY51 and SYN51 in blood samples at different time points were assessed by LC-MS/MS. Secondary PK parameters have been calculated from 2C model (bi-exponential kinetics).

TABLE 1

| Secondary PK | SQY51 | | SYN51 | |
|---|---|---|---|---|
| Parameters | Mean | CV % | Mean | CV % |
| α t½ (h) | 2.01 | 21.55 | 0.70 | 8.10 |
| β t½ (h) | 90.14 | 38.99 | 21.74 | 0.92 |
| AUC (μg/mL*h) | 58609.60 | 25.73 | 19406.10 | 9.15 |
| Cₘₐₓ (μg/mL) | 880.76 | 0.62 | 1018.76 | 7.35 |
| MRT (h) | 127.04 | 38.77 | 30.65 | 0.61 |
| VSS | 106.49 | 13.73 | 79.32 | 9.76 |

It is important to note that SQY51 remained stable throughout the elimination phase —Mass spectrometry analyzes all over the study revealed that SQY51 remained intact in the blood flow and retained its palmitoyl moiety.

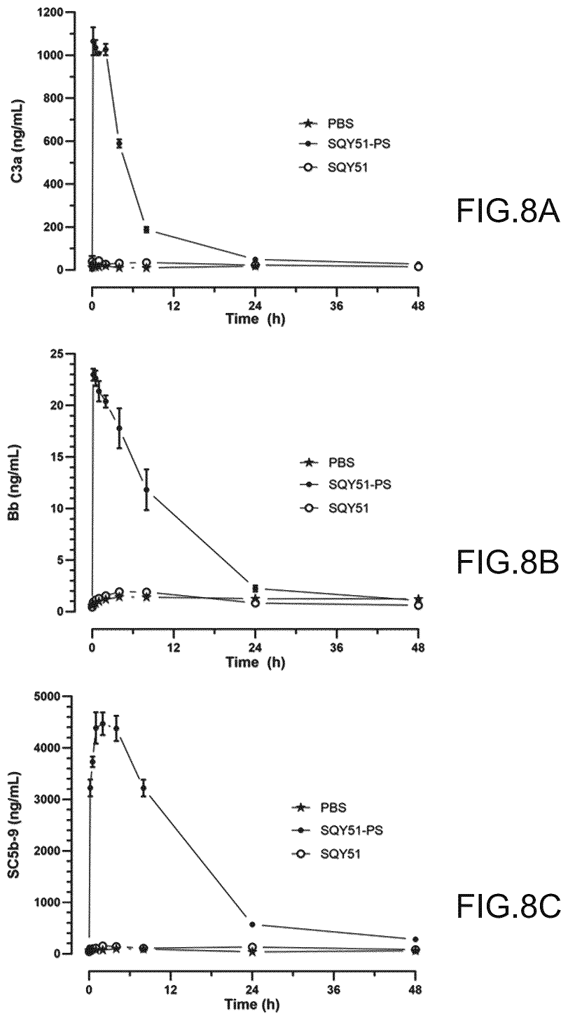

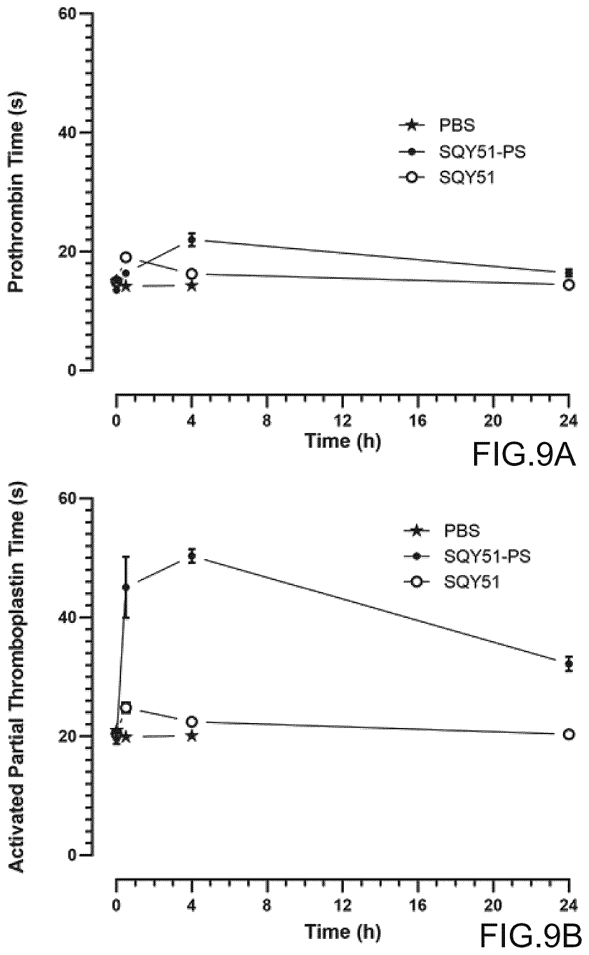

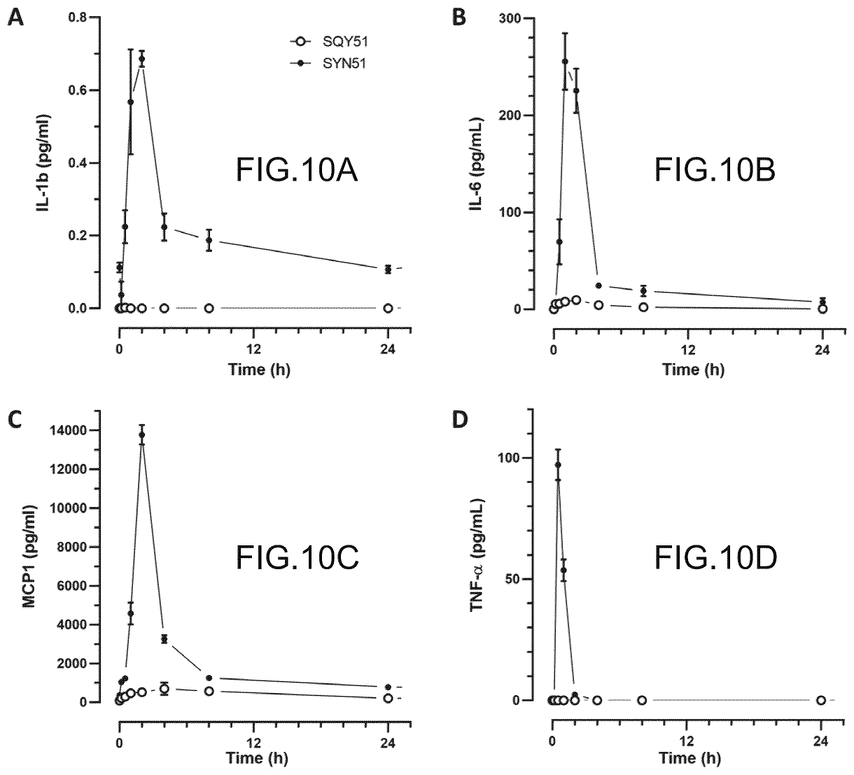

Given the high persistence of SQY51 in the blood, due to its special binding properties with primate-serum albumin, it was central to check whether such prolonged presence would trigger deleterious events such as complement activation (FIG. 8), coagulation misfunctioning (FIG. 9), and rising of pro-inflammatory cytokines (FIG. 10). None of these potentially harmful events were observed with SQY51, but they were observed when the compound contained PS-type internucleotide bonds.

Concerning the pro-inflammatory cytokines studied in FIG. 10, the interleukin 1 beta (IL-1β) is produced by activated macrophages and it is an important mediator of the inflammatory innate response. The interleukin 6 (IL-6), an inflammatory cytokine, can be secreted by macrophages in response to molecules referred to as pathogen-associated molecular patterns (PAMPs) that bind to detection molecules of the innate immune system, called pattern recognition receptors (PRRs), including Toll-like receptors (TLRs), that are present on the cell surface and intracellular compartments. Monocytes/macrophages are the major source of Monocyte Chemoattractant Protein 1 (MCP-1), although it can be produced by other cell types, including endothelial cells and fibroblasts, which are important for antiviral immune responses in the peripheral circulation and in tissues. Tumor Necrosis Factor alfa (TNF-α) is a pro-inflammatory cytokine naturally produced by activated macrophages and monocytes in response to infection and injury, which mediates hypotension, diffuse coagulation, and widespread tissue damage.

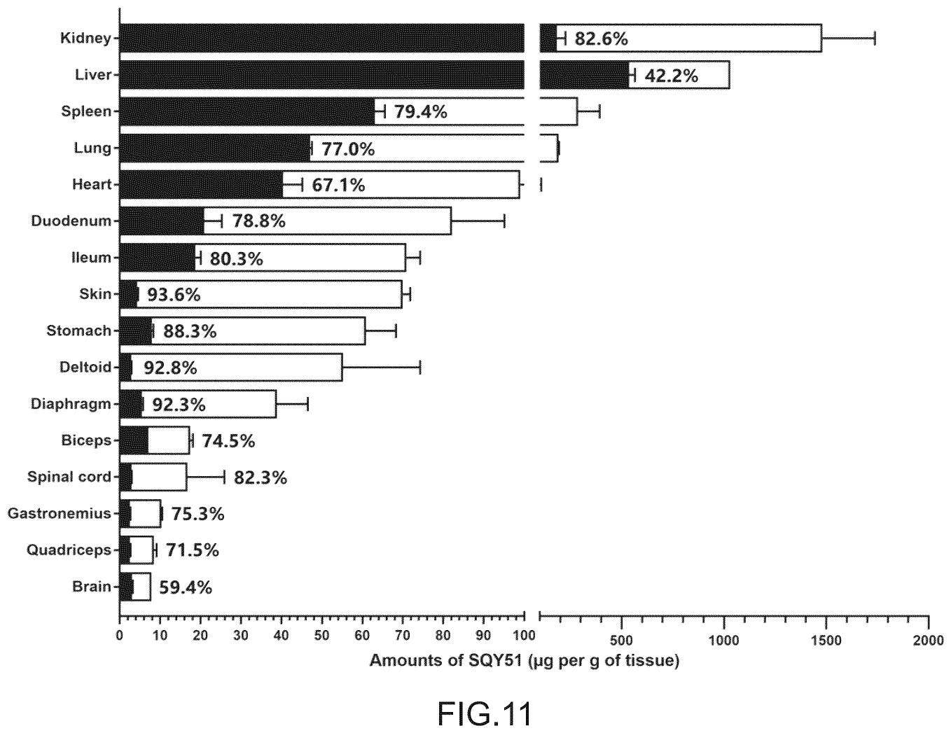

Analysis of the biodistribution of SQY51 in the various tissues of the body was carried out at one week and five weeks after four weekly-injections at a dose of 50 mg/kg (FIG. 11). First, it appears as expected that the kidney and liver are the main organs that accumulate the compound.

The below Table 2 shows comparison of exon-51 skipping in cynomolgus muscles one week after a 4-week treatment (50 mg/kg/week) with either SQY51 or SYN51. On average, SQY51 is 10 times more efficient than SYN51.

TABLE 2

| % exon-51 skipping | SQY51 | SYN51 |
|---|---|---|
| in striated muscles | Mean | Mean |
| Deltoid | 27.1 | 0.0 |
| Gastrocnemius | 16.3 | 1.3 |
| Quadriceps | 17.2 | 1.6 |
| Biceps | 16.8 | 1.9 |
| Diaphragm | 18.9 | 2.8 |
| Heart | 36.8 | 5.5 |
| AVERAGE | 22.2 | 2.2 |

Thus, among the striated muscles, the heart seems to be a prime target. Second, it is important to note that the amount of SQY51 decreased very significantly in tissues after only 4 additional weeks of wash-out, particularly in kidney. Indeed, a clearance of over 70% in all tissues after four weeks post-treatment was observed.

### Example 5: Efficacy of the Compound SQY51 in Cynomolgus Monkey

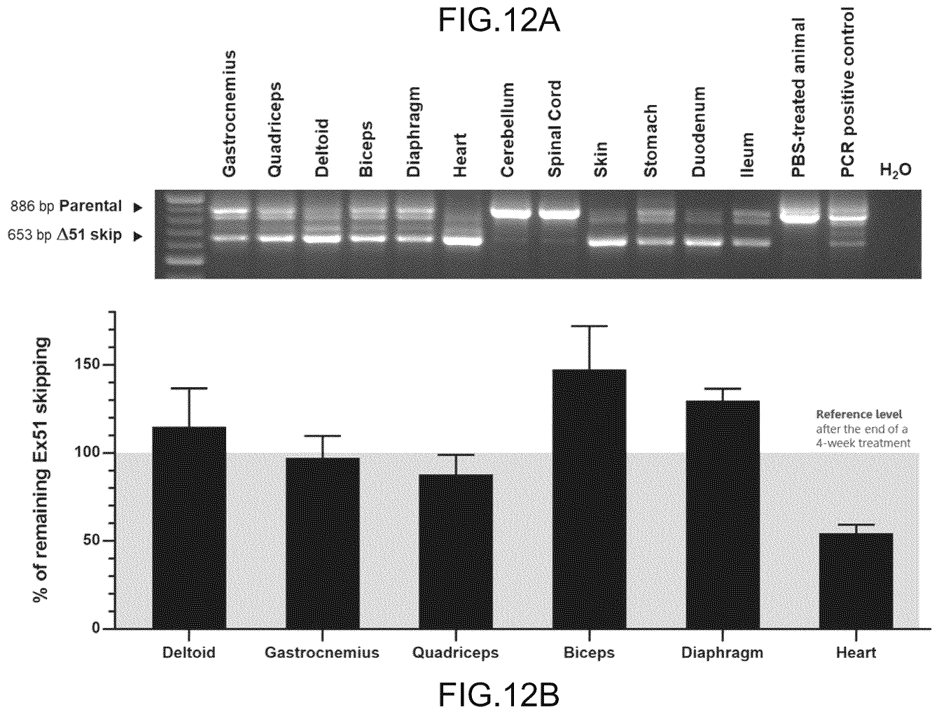

Nested RT-PCR analysis showed levels of exon-51 skipping in monkey tissues after systematic administration of SQY51 (FIG. 12), thus confirming widespread delivery of its antisense moiety to myo-nuclei of the whole musculature, including skeletal muscles, heart and smooth muscles.

Noteworthy, despite the high clearance rate of SQY51 in muscles (see FIG. 11), levels of exon 51 skipping at 5 weeks were relatively similar to those detected at 1 week, and thus are almost maintained regardless of the wash-out, indicating that the cleared compound was mostly an unproductive fraction of SQY51 possibly stranded in interstitial fluids or in endosomal compartments where it is cleared out or underwent destruction. Furthermore, this lasting effect indicates that treatment with SQY51 can be discontinued for periods longer than one or two months without significant loss of benefit, thus offering the body the time to get rid of putative excess of the tricyclo-DNA oligomer in tissues at risk such as the kidney and liver.

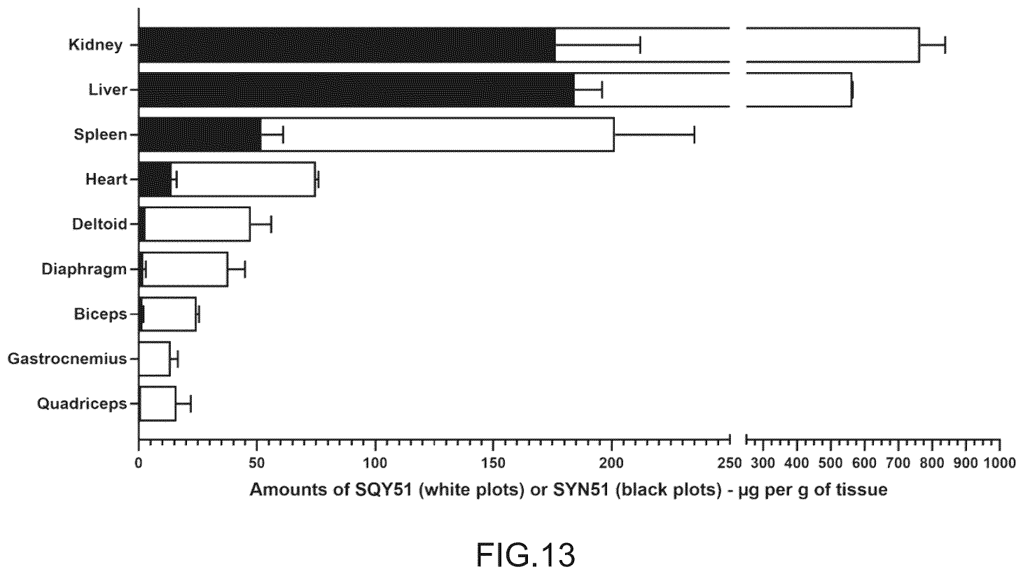

Finally, the novel compound SQY51 has crucial advantages over SYN51, which was so far considered as the finest tc-DNA-based compound for skipping the exon-51 in DMD: (i) because of its inner properties (e.g., higher specific binding to human & NHP serum albumin) SQY51 shows improved biodistribution in monkey muscles after systemic delivery (FIG. 13), and (ii) subsequent exon-51 skipping levels in striated muscles were 10 times higher, demonstrating that SQY51 is of real therapeutic interest.

1. An oligomeric compound comprising from 10 to 50 monomer subunits wherein at least part of the sequence of the oligomeric compound is complementary to the sequence:
(SEQ ID NO: 1)
AAGGAAACUGCCAUCUCCAA.

2. The oligomeric compound according to claim 1, wherein at least part of the sequence of the oligomeric compound is complementary to the sequence corresponding to the region +48+62 of SEQ ID NO: 2.

3. The oligomeric compound according to claim 1, wherein the oligomeric compound comprises an antisense oligonucleotide.

4. The oligomeric compound according to claim 1, wherein the oligomeric compound comprises at least one nucleotide sequence having at least 70% identity with the following tc-DNA nucleotide sequence:
(SEQ ID NO: 3)
GGAGATGGCAGTTTC.

5. The oligomeric compound according to claim 1, wherein the oligomeric compound comprises a tricyclo-DNA antisense oligonucleotide.

6. The oligomeric compound according to claim 1, wherein the oligomeric compound comprises a tricyclo-phosphorothioate DNA antisense oligonucleotide.

7. The oligomeric compound according to claim 1, wherein the oligomeric compound comprises one or more tricyclo-deoxyribonucleic acid (tc-DNA) nucleosides and at least one modified ribonucleic acid nucleoside.

8. The oligomeric compound according to claim 7, wherein the modified ribonucleic acid nucleoside is a 2′-O-methyl RNA nucleoside.

9. Tie oligomeric compound according to claim 7, wherein the monomer subunits of said oligomeric compound are joined by phosphodiester internucleoside linkages.

10. The oligomeric compound according to claim 7, wherein the oligomeric compound comprises a nucleotide sequence selected from:
(SEQ ID NO: 4)
5′-GGAGATgGCAGTTTC-3′,
(SEQ ID NO: 5)
5′-GGAGATGgCAGTTTC-3′,
(SEQ ID NO: 6)
5′-GGAGATGGcAGTTTC-3′,
and
(SEQ ID NO: 7)
5′-GGAGATGGCaGTTTC-3′.
in which tcDNA nucleotides are typed in capital letters while the modified ribonucleic acid nucleoside is typed in lowercase letter.

11. The oligomeric compound according to claim 1, wherein the oligomeric compound further comprises one or more conjugated lipid moieties.

12. The oligomeric compound according to claim 1, wherein the oligomeric compound is selected from:
(SEQ ID NO: 4)
palmitate-NH-C₆alkylene-OP(═S)(OH)-
GGAGATgGCAGTTTC-3′,
(SEQ ID NO: 5)
palmitate-NH-C₆alkylene-OP(═S)(OH)-
GGAGATGgCAGTTTC-3′,
(SEQ ID NO: 6)
palmitate-NH-C₆alkylene-OP(═S)(OH)-
GGAGATGGcAGTTTC-3′,
and
(SEQ ID NO: 7)
palmitate-NH-C₆alkylene-OP(═S)(OH)-
GGAGATGGCaGTTTC-3′,
in which tcDNA nucleotides are typed in capital letters while the modified ribonucleic acid nucleoside is typed in lowercase letter.

13. A pharmaceutical composition comprising an oligomeric compound according to claim 1 and a pharmaceutically acceptable vehicle.

14. (canceled)

15. (canceled)

16. The oligomeric compound according to claim 8, wherein the one or more lipid moieties is a fatty acid moiety, a fatty diacid moiety, a glycerolipid moiety, a glycerophospholipid moiety, a sphingolipid moiety, a phospholipid, an alkylphosphate moiety or an alkylphosphonate moiety.

17. The oligomeric compound according to claim 8, wherein the one or more lipid moiety is a saturated fatty acid moiety derived from palmitoleic acid.

18. A method for treating Duchenne Muscular Dystrophy in a subject in need thereof by administering to the subject a therapeutically effective amount of a composition comprising the oligomeric compound according to claim 1.

19. A method for treating Duchenne Muscular Dystrophy in a subject in need thereof by administering to the subject a therapeutically effective amount of an oligomeric compound selected from:
(SEQ ID NO: 4)
palmitate-NH-C₆alkylene-OP(═S)(OH)-
GGAGATgGCAGTTTC-3′,
(SEQ ID NO: 5)
palmitate-NH-C₆alkylene-OP(═S)(OH)-
GGAGATGgCAGTTTC-3′,
(SEQ ID NO: 6)
palmitate-NH-C₆alkylene-OP(═S)(OH)-
GGAGATGGcAGTTTC-3′,
and
(SEQ ID NO: 7)
palmitate-NH-C₆alkylene-OP(═S)(OH)-
GGAGATGGCaGTTTC-3′
in which tcDNA nucleotides are typed in capital letters while the modified ribonucleic acid nucleoside is typed in lowercase letter.
